(19) United States
(12) Patent Application Publication
(10) Pub. No.: US 2013/0273057 A1
(43) Pub. Date: Oct. 17, 2013

(54) METHODS AND COMPOSITIONS FOR THE TREATMENT AND DIAGNOSIS OF VASCULAR INFLAMMATORY DISORDERS OR ENDOTHELIAL CELL DISORDERS

(71) Applicant: Beth Israel Deaconess Medical Center, Inc., Boston, MA (US)

(72) Inventors: Vikas P. Sukhatme, Newton, MA (US); Chunkong Barden Chan, Allston, MA (US)

(21) Appl. No.: 13/849,871

(22) Filed: Mar. 25, 2013

Related U.S. Application Data
(63) Continuation of application No. 13/155,962, filed on Jun. 8, 2011, now abandoned.
Application No. 13/155,962 is a continuation of application No. 12/592,034, filed on Nov. 18, 2009, now abandoned.
Application No. 12/592,034 is a continuation of application No. 12/008,663, filed on Jan. 11, 2008, now abandoned.
(60) Provisional application No. 60/879,908, filed on Jan. 11, 2007.

(51) Int. Cl. A61K 39/395; A61K 38/45; A61K 31/7105

(52) U.S. Cl. CPC A61K 39/3955; A61K 31/7105; A61K 38/45
USPC 424/139.1; 514/44 A; 514/1.9

(57) ABSTRACT

Disclosed herein are methods for treating a vascular inflammatory disorder or endothelial cell disorder using inhibitor compounds that inhibit the expression or biological activity of Tie-1, Tie-1 endodomain, thrombin, VEGFR², VEGFR² endodomain, EphA2, and any of the cytokines or kinases that are upregulated by activation of Tie-1 or thrombin, as provided herein. Also disclosed are the use of combinations of inhibitor compounds or the use of an eNOS activator compound in combination with any one or more of the inhibitor compounds. Also disclosed are methods for inhibiting the pro-coagulant activity of thrombin using a Tie-1 or Tie-1 endodomain inhibitor compound or an EphA2 inhibitor compound. Methods for diagnosing and monitoring vascular inflammatory disorders or endothelial cell disorders that include the measurement of any of the polypeptides or nucleic acid molecules of the invention are also disclosed.

### CROSS-REFERENCE TO RELATED APPLICATIONS

This application is a continuation of U.S. patent application Ser. No. 13/155,962, filed on Jun. 8, 2011, which is a continuation of U.S. patent application Ser. No. 12/592,034, filed on Nov. 18, 2009, which is a continuation of U.S. patent application Ser. No. 12/008,663, filed on Jan. 11, 2008, which claims the benefit of the filing date of U.S. Provisional Application No. 60/879,908, filed on Jan. 11, 2007, each of which is hereby incorporated by reference.

### BACKGROUND OF THE INVENTION

In general, the invention relates to methods and compositions for the treatment and diagnosis of vascular inflammatory disorders and endothelial cell disorders.

Endothelial cell health is critical to the maintenance of vascular health and vascular diseases are often caused by injury to the endothelial cells. Endothelial cell disorders include any disorder that is characterized by endothelial cell dysfunction. The most common form of endothelial cell disease is vascular inflammatory disorders. Vascular inflammatory disorders are characterized not only by endothelial cell dysfunction but also have a smooth muscle cell component as the vasculature is made up a variety of cell types including endothelial cells and smooth muscle cells.

One example of a vascular inflammatory disorder is arteriosclerosis. Arteriosclerosis is a generic term for a number of diseases in which the arterial wall becomes thickened and loses elasticity. Upon injury to the arterial endothelium, large molecules (e.g., macrophages, lipid, and cholesterol) are allowed to escape through the endothelium and form deposits in the smooth muscle cells in the arterial wall. Macrophages also pass through and accumulate fat (lipid and cholesterol) deposits. This process is very slow, but there is a gradual accumulation of this fatty and fibrous material which not only makes the normally elastic artery sclerotic but the deposits, known as “plaques,” may lead to a narrowing of the artery and facilitate the formation of a blood clot or a thrombosis. Myocardial infarction and stroke are additional consequences that result from endothelial cell injury or a disruption to the endothelial layers of the arteries.

Atherosclerosis, a subset of arteriosclerosis, is a disease of the arteries characterized by fatty deposits on the intimal or inner lining of the arteries. In the United States and most other Western countries, atherosclerosis is a major health problem and one of the leading causes of illness and death.

In atherosclerosis, the presence of fatty deposits and fibrous plaques leads to an important loss of arterial elasticity with narrowing of the artery. This constriction to smooth blood-flow ultimately deprives vital organs of their blood supply. Atherosclerosis can affect the medium-sized and large arteries of the brain, heart, kidneys, other vital organs, and legs. Clots may lodge in arteries supplying the heart, causing myocardial infarction (heart attack), or the brain, causing stroke.

Atherosclerosis is thought to also involve inflammation, because certain white blood cells—lymphocytes, monocytes, and macrophages—are present throughout the development of atherosclerosis. These cells usually gather only when inflammation develops. Atherosclerosis begins when monocytes are activated and move out of the bloodstream into the wall of an artery. There, they are transformed into foam cells, which collect cholesterol and other fatty materials. In time, these fat-laden foam cells accumulate and form patchy deposits (atheromas) in the lining of the arterial wall, causing a thickening of the artery. A brief review of the initial steps leading to atherosclerosis lesion formation is given below.

Low-density lipoprotein (LDL) uptake by the arterial wall is a key step in atherosclerosis development. LDL is modified in the intima and one important modification is oxidation by reactive oxygen species which then induces an inflammatory response in the endothelial cells. Adhesion molecules such as ICAM-1 and VCAM-1 are upregulated on the endothelial surface. The activated endothelial cells also secrete proinflammatory molecules, such as the macrophage chemoattractant protein-1 (MCP-1) and the macrophage colony-stimulating factor (M-CSF). These cytokines and adhesion molecules aid in the recruitment and transendothelial migration of monocytes. In the intima, the monocytes differentiate into scavenger-receptor-expressing macrophages. These macrophages internalize oxidized LDL and become foam cells, which produce additional proinflammatory molecules, resulting in amplification of the inflammatory response.

T-lymphocytes are also recruited to the sites of atherosclerosis and play an important part in the development of the disease. Facilitated by the adhesion molecules expressed on the surface of activated endothelial cells, T-cells adhere to and transmigrate through the endothelium. The transendothelial migrated T-cells are activated by antigens present in the intima such as oxidized LDL. In addition, CD154 present in the T-cells can interact with its ligand CD40 expressed by macrophages. These events collectively result in secretion of additional proinflammatory cytokines by the T-cells.

In response to the proinflammatory cytokines and growth factors produced by macrophages, T-cells, and activated endothelial cells, smooth muscle cells become activated as well and migrate from the media to the intima. Activated smooth muscle cells proliferate and secrete proinflammatory cytokines and extracellular matrix proteins in the intima, contributing to the development of inflammation.

All of these events, when taken together, result in the formation of fatty deposits and fibrotic plaques leading to a narrowing of the arteries or arthrosclerosis.

Tie-1 receptor is an endothelial specific cell surface tyrosine kinase that is indispensable for endothelial functions. However, a high affinity binding, signaling ligand has not been conclusively identified for Tie-1 and very little is known about the specific biology of this molecule. Although Tie-1 expression has been detected in a number of pathological conditions, the function of Tie-1 in normal or pathological conditions remains unknown. Moreover, there have even been conflicting reports regarding the kinase activity of Tie-1 and the mechanism of Tie-1 activation. Tie-1 has been shown to undergo a cleavage event upon activation which results in shedding of the Tie-1 ectodomain generating a membrane-bound Tie-1 endodomain. However, the activity of the Tie-1 endodomain remains unknown.

Thrombin is a multifunctional serine protease that is a coagulation protein that has numerous effects on the coagulation cascade. Thrombin converts fibrinogen into insoluble fibrin and also activates factor XI, factor V, and factor VIII, which are also involved in the activation of thrombin from prothrombin resulting in a positive feedback look that accelerates the production of thrombin. Thrombin is also known to play a critical role in endothelial biology, however the exact role of thrombin and the downstream regulators of thrombin signaling in vascular endothelial cells remain unknown. Although the procoagulation activities of thrombin are well-characterized, the role for thrombin in vascular endothelial cell health remains unclear. To date, there has been no evidence for a connection between thrombin and Tie-1 in endothelial cells or for a pathological role for thrombin and Tie-1 in the development of endothelial cell dysfunction or vascular inflammatory disorders. In addition, although the importance of thrombin in vascular lesion development has been suggested, these studies have only focused on the effect of thrombin on either smooth muscle cells or macrophages with respect to vascular lesion development. Very little is known about the involvement and consequences of endothelial activation by thrombin in this pathological state.

While great progress has been made in the identification of risk factors for vascular inflammatory disorders, the molecular mechanisms that trigger the initiation of disorders, such as atherosclerosis, remain unclear. Diagnostic tools and therapeutics for vascular inflammatory disorders, such as atherosclerosis, are needed to reduce the significant morbidity and mortality associated with these disorders.

### SUMMARY OF THE INVENTION

Endothelial cell health is critical to the maintenance of vascular cell health and to the prevention of vascular diseases including arteriosclerosis and atherosclerosis. Endothelial cell health is also critical for the treatment or prevention of endothelial cell dysfunction and endothelial cell disorders characterized by endothelial cell dysfunction.

We have shown that the Tie-1 endodomain is biologically active and, using the active Tie-1 endodomain or overexpressing the full length Tie-1, we have discovered that Tie-1 is a critical upstream regulator of pathways that are associated with endothelial cell dysfunction and vascular inflammatory disorders, such as atherosclerosis. We have discovered that Tie-1 stimulates expression of the cytokine markers IP-10, G-CSF, IL-6, VCAM-1, ICAM-1, CCL20, CCL2, CXCL5, E-selectin, p38 MAP kinase, and soluble CD44. Tie-1 also down-regulates endothelial nitric oxide synthase (eNOS) expression. In addition, we have discovered that Tie-1 regulates the expression or biological activity of the genes indicated in Appendices of U.S. Provisional Application No. 60/879,908, filed on Jan. 11, 2007, herein incorporated by reference (hereafter referred to as “the Appendix”) or the proteins encoded by these genes. We have also discovered that Tie-1 enhances attachment of monocytes to endothelial cells and enhances smooth muscle cell migration. Moreover, activated Tie-1 stimulates the expression or biological activity of tissue factor and thrombin. The ability of Tie-1 to upregulate the expression or biological activity of these cytokines and coagulation factors combined with its ability to induce monocyte attachment and smooth muscle cell proliferation indicates that Tie-1 is an upstream regulator of many of the pathways known to be involved in the development of vascular inflammatory disorders and endothelial cell disorders. Accordingly, therapeutic compounds that inhibit Tie-1 or polypeptides shown to be upregulated in the presence of activated Tie-1 can be used for the treatment or prevention of vascular inflammatory disorders and endothelial cell disorders.

Thrombin is another molecule that has been associated with vascular lesions, however, the exact effects of thrombin on endothelial cells is unclear because thrombin is known to influence many cell types in the vasculature. We have discovered that expression of activated Tie-1 promotes an increase in the expression of a number of pro-inflammatory cytokines (e.g., Tie-2, tissue factor, IP-10, G-CSF, IL-6, VCAM-1, ICAM-1, CCL20, CCL2, CXCL5, E-selectin, soluble CD44, p38 MAP kinase) can activate thrombin in an endothelial-cell specific manner, which in turn stimulates endothelial cells through PAR-1 and transactivates Tie-1. This scenario results in an amplification loop of endothelial inflammation which may trigger the onset of a vascular inflammatory disorder or an endothelial cell disorder. We have also discovered that thrombin activates a number of receptor tyrosine kinases (e.g., p38 MAP kinase, EGFR, insulin receptor, IGF-IR, AXL, HGFR, Flt-1, KDR, c-RET, MER, and EphA2) in endothelial cells.

Therefore, according to the present invention, therapeutic compounds that inhibit Tie-1, thrombin, tissue factor, or any of the upregulated tyrosine kinases, particularly in endothelial cells, can be used to treat vascular inflammatory disorders or endothelial cell disorders. Furthermore, since we have discovered that thrombin is downstream of Tie-1 in endothelial cell signaling pathways, Tie-1 inhibitor compounds and/or compounds that inhibit the upregulated tyrosine kinases in a cell or a subject in need thereof can be used to specifically inhibit the pro-inflammatory effects of thrombin without interfering with the ability of thrombin to promote fibrin conversion and clot formation.

One of the tyrosine kinases that we discovered to be activated by thrombin stimulation of endothelial cells was vascular endothelial growth factor receptor-2 or VEGFR-2 (also known as KDR). VEGFR2 was activated in a VEGF-independent manner and a previously unidentified truncated form of VEGFR2 was identified. We have shown that this newly discovered truncated form, which we termed the VEGFR2 endodomain, results from receptor cleavage and shedding of the VEGFR2 ectodomain. The VEGFR2 endodomain has a molecular weight of approximately 120 kD, is detected by antibodies that specifically bind to the carboxy terminus of VEGFR2, and is phosphorylated in its activated form. Therefore, the invention also features VEGFR2 endodomain compositions that are useful for promotion of vascular or lymph endothelial cell growth and VEGFR2 endodomain specific inhibitor compounds that are useful for the treatment or prevention of angiogenic disorders, endothelial cell disorders, or vascular inflammatory disorders.

Another one of the tyrosine kinases that we discovered to be activated by thrombin stimulation of endothelial cells was EphA2. Ephrin-A1 was first identified as an immediate-early response gene of endothelial cells induced by inflammatory stimuli such as TNF-α, IL-1β, and lipopolysaccharide; however, very little was known about the specific functions of the Eph receptors/Ephrins in vascular inflammation. Our discoveries have shown that EphA2 is a downstream mediator of thrombin and the activation of EphA2 by thrombin is rapid and is independent of EphA2 cognate ligands, such as Ephrin A1. We have demonstrated that EphA2 is required for thrombin-induced ICAM-1 upregulation in endothelial cells. Furthermore, we have discovered that downregulation of EphA2 potently reduces leukocyte attachment to thrombin-stimulated endothelial cells in vitro. These discoveries provide a novel link between EphA2 and the effects of thrombin on endothelial cell biology and vascular inflammation. The invention also features EphA2 inhibitor compounds that are useful for the treatment or prevention of vascular inflammatory disorders and endothelial cell disorders.

Accordingly, in one aspect, the invention features a method of treating or preventing a vascular inflammatory disorder in a subject that includes administering to the subject a therapeutically effective amount of a Tie-1 inhibitor compound in an amount and for a time sufficient to treat or prevent the vascular inflammatory disorder in the subject.

In another aspect, the invention features a method of treating or preventing a vascular inflammatory disorder in a subject, where the method includes administering to the subject a therapeutically effective amount of a EphA2 inhibitor compound in an amount and for a time sufficient to treat or prevent the vascular inflammatory disorder.

In yet another aspect, the invention features a method of treating or preventing an endothelial cell disorder in a subject where the method includes administering to the subject a therapeutically effective amount of a Tie-1 inhibitor compound or an EphA2 inhibitor compound in an amount and for a time sufficient to treat or prevent the endothelial cell disorder in the subject.

In yet another aspect, the invention features a method of inhibiting thrombin biological activity in a cell, wherein the cell can be in vitro or in vivo (e.g., in a subject), and the method includes contacting the cell with a Tie-1 inhibitor compound in an amount and for a time sufficient to inhibit thrombin biological activity. In one example, the cell is in a subject and the Tie-1 inhibitor compound is administered to the subject. Desirably, the Tie-1 inhibitor compound inhibits the pro-inflammatory activity of thrombin and does not inhibit the thrombin-mediated conversion of fibrinogen to fibrin.

In yet another aspect, the invention features a method of inhibiting thrombin biological activity in a cell, wherein the cell can be in vitro or in vivo (e.g., in a subject), and the method includes contacting the cell with an EphA2 inhibitor compound in an amount and for a time sufficient to inhibit thrombin biological activity. In one example, the cell is in a subject and the EphA2 inhibitor compound is administered to the subject. Desirably, the EphA2 inhibitor compound inhibits the pro-inflammatory activity of thrombin and does not inhibit the thrombin-mediated conversion of fibrinogen to fibrin.

The Tie-1 inhibitor compound or EphA2 inhibitor compound can also be used in combination with any compound that reduces or inhibits the activity or expression levels of thrombin, tissue factor, or any of the cytokines that we discovered are upregulated in the presence of activated or overexpressed Tie-1 (e.g., those described herein or listed in the Appendix). In addition, compounds that are found to upregulate any of the genes that are identified in the Appendix as downregulated in the presence of Tie-1 (e.g., eNOS), can also be used for the treatment or prevention of vascular inflammatory disorders.

The invention also features the use of any combination of a Tie-1 inhibitor compound and one or more of the following: any compound that inhibits the activity of the cytokines or adhesion molecules that are upregulated by Tie-1 (described herein or in the Appendix), any compound that enhances the activity of the cytokines or adhesion molecules that are downregulated by Tie-1 (described herein or in the Appendix, e.g., eNOS), a tissue factor inhibitor compound, a thrombin inhibitor compound, and an eNOS activator compound. For example, a tissue factor inhibitor compound can be used in combination with a cytokine or adhesion marker inhibitor (e.g., an inhibitor of G-CSF or VCAM-1) to treat a vascular inflammatory disorder. In another example, inhibitors or activators of two, three, four, five, six or more of the cytokine or adhesion markers that we have discovered are upregulated or downregulated in the presence of Tie-1 can be used together to treat a vascular inflammatory disorder or endothelial cell disorder.

In another aspect, the invention features a method of treating or preventing a vascular inflammatory disorder or endothelial cell disorder in a subject, that includes administering to the subject a therapeutically effective amount of two or more compounds that inhibit the biological activity or expression level of at least two of the following proteins: Tie-1, Tie-2, tissue factor, thrombin, IP-10, G-CSF, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, KDR, VEGFR2 endodomain, c-RET, MER, and EphA2. Optionally, the method can also include administering to the subject a compound that increases the expression level or biological activity of nitric oxide synthase (eNOS).

The invention also features kits including the Tie-1 inhibitor compound, the EphA2 inhibitor compound or any one or more of the inhibitor or activator compounds of the invention, or any combination thereof, for use in the treatment of a vascular inflammatory disorder an endothelial cell disorder or for inhibition of thrombin biological activity.

In yet another aspect, the invention features a method of treating or preventing pre-eclampsia or eclampsia in a subject in need thereof, where the method includes administering to the subject an EphA2 inhibitor compound in an amount and for a time sufficient to treat or prevent the pre-eclampsia or eclampsia in the subject.

For any of the aspects of the invention, the Tie-1 inhibitor compound is a compound that reduces or inhibits the biological activity or expression levels of a Tie-1 protein or nucleic acid molecule. Non-limiting examples of Tie-1 biological activity include kinase activity; cleavage of Tie-1; shedding of the Tie-1 ectodomain; phosphorylation of the Tie-1 endodomain; increased endothelial cell adhesion; increased smooth muscle cell migration; inhibition of eNOS expression or biological activity; and activation of one or more cytokine or inflammatory markers (e.g, thrombin, tissue factor, G-CSF, IL-6, IP-10, VCAM-1, ICAM-1, CCL20, CCL2, CXCL5, E-selectin, soluble CD44, and p38 MAP kinase).

In one example, the Tie-1 inhibitor compound is a polypeptide that specifically binds Tie-1, for example the Tie-1 endodomain, or the ATP binding pocket of Tie-1. Non-limiting examples of such a polypeptide include an antibody or antigen-binding fragment thereof (e.g., including a monoclonal antibody, a polyclonal antibody, a single-chain antibody, a chimeric antibody, a humanized antibody, a fully humanized antibody, a human antibody, and a bispecific antibody), a dominant negative Tie-1 polypeptide that does not induce Tie-1 biological activity, or an antagonistic ligand that binds to but does not activate Tie-1 signaling.

In another example, the Tie-1 inhibitor compound is a nucleobase oligomer that reduces or inhibits the expression of a Tie-1 polypeptide or nucleic acid molecule. Non-limiting examples include an antisense nucleobase oligomer (e.g., 8 to 30 nucleotides) complementary to at least a portion of a Tie-1 nucleic acid molecule; a morpholino oligomer that is complementary to at least a portion of a Tie-1 nucleic acid molecule; a small RNA (e.g., a double stranded RNA that is processed into small interfering RNAS (siRNAs) 19 to 25 nucleotides in length) that includes a nucleic acid sequence that is substantially identical to at least a portion of an Tie-1 nucleic acid sequence, or a complementary sequence thereof.

For any of the above aspects, the EphA2 inhibitor compound reduces or inhibits the biological activity or expression levels of a EphA2 protein or nucleic acid molecule. Non-limiting examples of the biological activity of an EphA2 protein includes ligand binding; kinase activity; Ephrin A1 independent kinase activity; interaction with an SH2 domain containing signaling proteins (e.g., CrkL, SHP-2, the α subunit of PI3K, and the β subunit of PI3K); ICAM-1 upregulation (including NFkB dependent ICAM-upregulation); leukocyte attachment; and regulation of angiogenesis. In one example, the EphA-2 inhibitor compound blocks ICAM-1 upregulation (including NFkB dependent ICAM-1 upregulation).

In one example, the EphA2 inhibitor compound is a polypeptide that specifically binds EphA2, for example at the ATP binding pocket or at a phosphorylated tyrosine on EphA2. Non-limiting examples of such a polypeptide include an antibody or antigen-binding fragment thereof (e.g., including a monoclonal antibody, a polyclonal antibody, a single-chain antibody, a chimeric antibody, a humanized antibody, a fully humanized antibody, a human antibody, and a bispecific antibody), a dominant negative EphA2 polypeptide that does not induce EphA2 biological activity, or an antagonistic ligand that binds to but does not activate EphA2 signaling.

In another example, the EphA2 inhibitor compound is a nucleic acid molecule (e.g., nucleobase oligomer) that reduces or inhibits the expression of an EphA2 polypeptide or nucleic acid molecule. Non-limiting examples include an antisense nucleobase oligomer (e.g., 8 to 30 nucleotides) complementary to at least a portion of an EphA2 nucleic acid molecule; a morpholino oligomer that is complementary to at least a portion of a EphA2 nucleic acid molecule; a small RNA (e.g., a double stranded RNA that is processed into small interfering RNAS (siRNAs) 19 to 25 nucleotides in length) that includes a nucleic acid sequence that is substantially identical to at least a portion of an EphA2 nucleic acid sequence, or a complementary sequence thereof.

The invention also includes the use of Tie-1, thrombin, the VEGFR2 endodomain, marker proteins that were identified as activated or upregulated in the presence of active or overexpressed Tie-1 (e.g., ICAM-1, VCAM-1, IL-6, GCSF, tissue factor, CCL20, CCL2, CXCL5, soluble (alternatively spliced) CD44, E-selectins, and p38 MAP kinase, marker proteins that were identified as inactive or downregulated in the presence of active Tie-1 (e.g., eNOS) and endothelial cell tyrosine kinase receptor proteins that were identified as elevated or activated in the presence of thrombin (e.g., EGFR, insulin receptor, IGF-IR, AXL, HGFR (c-met), Flt-1, KDR, c-RET, MER, EphA2, Tie-1, and Tie-2) for the diagnosis of vascular inflammatory or endothelial cell disorders, such as atherosclerosis, or a risk of developing a vascular inflammatory or endothelial cell disorder. For the diagnostic methods of the invention, either the polypeptide levels or the nucleic acid levels can be measured in a subject sample (e.g., a bodily fluid, cell, or tissue) using methods known in the art (e.g., immunological assay, enzymatic assay, colorimetric assay for polypeptides and PCT, Southern, northern blot assays for nucleic acids). The levels of the nucleic acid or polypeptide can be compared to an absolute threshold level or reference level which is a known indicator of vascular inflammatory or endothelial cell disorders. The levels of the nucleic acid or polypeptide can also be compared to the level from a normal reference sample wherein an increase in the level for the activated proteins and a decrease in the level for a downregulated protein is diagnostic of a vascular inflammatory or endothelial cell disorder. Desirably, the level of more than one polypeptide or nucleic acid is measured. In one embodiment, the levels of the more than one polypeptide are compared using a metric. These proteins and nucleic acid molecules can also be used to monitor the therapeutic efficacy of compounds, including compounds of the invention, used to treat the vascular inflammatory disorder, such as atherosclerosis.

For any of the above aspects, the vascular inflammatory disorder can be any disorder characterized by one or more of the following characteristics: endothelial cell dysfunction, angiogenesis, smooth muscle cell proliferation, inflammation, calcification, and a pro-coagulatory process. Non-limiting examples of vascular inflammatory disorders include arteriosclerosis, atherosclerosis, or neointimal hyperplasia.

For any of the above aspects, the endothelial cell disorder can be any disorder characterized by endothelial cell dysfunction. Non-limiting examples include cancer, infectious diseases, autoimmune disorders, vascular malformations, DiGeorge syndrome, HHT, cavernous hemangioma, transplant arteriopathy, vascular access stenosis associated with hemodialysis, vasculitis, vasculitidis, vascular inflammatory disorders, atherosclerosis, obesity, psoriasis, warts, allergic dermatitis, scar keloids, pyogenic granulomas, blistering disease, Kaposi sarcoma, persistent hyperplastic vitreous syndrome, retinopathy of prematurity, choroidal neovascularization, macular degeneration, diabetic retinopathy, ocular neovascularization, primary pulmonary hypertension, asthma, nasal polyps, inflammatory bowel and periodontal disease, ascites, peritoneal adhesions, contraception, endometriosis, uterine bleeding, ovarian cysts, ovarian hyperstimulation, arthritis, rheumatoid arthritis, chronic articular rheumatism, synovitis, osteoarthritis, osteomyelitis, osteophyte formation, sepsis, and vascular leak. In one example, the endothelial cell disorder is sepsis, vascular leak, or rheumatoid arthritis.

The invention also features compositions that include the VEGFR2 endodomain and post-translation modifications thereof, including the active phosphorylated form. The compositions can be VEGFR2 endodomain fusion proteins where the VEGFR2 endodomain is fused to another polypeptide, such as an Fc fusion to increase stability of the protein or a tag polypeptide sequence for detection. In addition, the invention provides a composition comprising biologically active VEGFR2 endodomain and a pharmaceutically acceptable carrier. The VEGFR2 endodomain protein can include a protein that has a molecular weight of about 90-150 kDA and where in the amino acid sequence of the polypeptide includes a sequence that is substantially identical to the carboxy-terminus of VEGFR2 and wherein the VEGFR2 endodomain can be detected using an antibody directed to the carboxy-terminus of VEGFR. The VEGFR2 is not the full length VGEFR2 and is desirably at least 90%, preferably 95%, 96%, 97%, 98%, 99% or 100% identical to amino acids 700 to 1356 of SEQ ID NO: 1.

In another aspect, the invention features a VEGFR2 endodomain nucleic acid molecule where the nucleic acid molecule encodes a VEGFR2 endodomain protein. In one embodiment, the nucleic acid molecule encodes a protein that is not the full length VGEFR2 and that is at least 90%, preferably 95%, 96%, 97%, 98%, 99% or 100% identical to amino acids 700 to 1356 of SEQ ID NO: 1. In another example, the nucleic acid molecule includes nucleic acids that are at least 90%, preferably 95%, 96%, 97%, 98%, 99% or 100% identical nucleotides 2100 to 4071 of SEQ ID NO: 2.

In another aspect, the invention provides a pharmaceutical composition useful for promotion of vascular or lymph endothelial cell growth comprising a therapeutically effective amount of the VEGFR2 endodomain in a pharmaceutically acceptable carrier. In another aspect, this composition further comprises another cell growth factor such as VEGF and/or PDGF, or fragments thereof.

The invention also features a method of promoting survival, proliferation, or migration of an endothelial cell that includes contacting the cell with a VEGFR2 endodomain polypeptide or a nucleic acid molecule encoding a VEGFR2 endodomain polypeptide.

The invention also features a method of inducing angiogenesis, vasculogenesis, endothelial cell permeability or inflammation in a subject in need thereof. This method includes administering to the subject a VEGFR2 endodomain polypeptide or nucleic acid molecule encoding a VEGFR endodomain polypeptide.

The VEGFR2 endodomain protein, or pharmaceutical compositions that include the VEGFR2 endodomain protein, can include a protein that has a molecular weight of about 90-150 kDA and where in the amino acid sequence of the polypeptide includes a sequence that is substantially identical to the carboxy-terminus of VEGFR2 and wherein the VEGFR2 endodomain can be detected using an antibody directed to the carboxy-terminus of VEGFR. The VEGFR2 is not the full length VGEFR2 and is desirably at least 90%, preferably 95%, 96%, 97%, 98%, 99% or 100% identical to amino acids 700 to 1356 of SEQ ID NO: 1.

In one example, the VEGFR2 endodomain polypeptide or nucleic acid molecule is used to treat a subject suffering from Alzheimer's disease, amyotrophic lateral sclerosis, diabetic neuropathy, stroke, diabetes, ulcers, restenosis, coronary artery disease, peripheral vascular disease, vascular leak, vasculitis, vasculitidis, injuries or wounds of the blood vessels or heart, Wegner's disease, gastric or oral ulcerations, cirrhosis, hepatorenal syndrome, Crohn's disease, hair loss, skin purpura, telangiectasia, venous lake formation, delayed wound healing, pre-eclampsia, eclampsia, ischemia-reperfusion injury, acute renal failure, hypertension, chronic or acute infection, menorrhagia, neonatal respiratory distress, pulmonary fibrosis, emphysema, nephropathy, hemolytic uremic syndrome, sclerodoma, and vascular abnormalities. In another example, the VEGFR2 endodomain polypeptide or nucleic acid molecule is used to treat a burn victim. In this example, the VEGFR2 endodomain polypeptide or nucleic acid molecule is used to prepare the burn site for a skin graft or flap.

The invention also features inhibitor compounds and compositions that include inhibitor compounds (e.g., antagonists) that specifically inhibit or reduce the biological activity or expression of the VEGFR2 endodomain, including the active phosphorylated form. The compositions can be compounds (peptidyl or non-peptidyl), small molecules, nucleic acids, or otherwise. In one example, the composition is an antagonistic antibody or polypeptide that specifically binds to the VEGFR2 endodomain and not the full-length VEGFR2. In addition, the invention provides a composition comprising a VEGFR2 endodomain specific inhibitor and a pharmaceutically acceptable carrier. Such compositions are useful for reducing or inhibiting angiogenesis, vasculogenesis, pseudovasculogenesis, vessel co-option, survival of endothelial cells, proliferation of endothelial cells, migration of endothelial cells, endothelial permeability, and inflammation. Such compositions can also be used in any of the methods of the invention described herein.

By “alteration” is meant a change (i.e., increase or decrease). The alteration can indicate a change in the expression levels of a nucleic acid or polypeptide of the invention as detected by standard art known methods such as those described below. As used herein, an alteration includes a 10% change in expression levels, preferably a 25% change, more preferably a 40%, 50%, 60%, 70%, 80%, 90%, 95%, 96%, 97%, 98%, 99%, or greater change in expression levels. The alteration can also indicate a change (i.e., increase or decrease) in the biological activity of a nucleic acid or polypeptide of the invention. As used herein, an alteration includes a 10% change in biological activity, preferably a 25% change, more preferably a 40%, 50%, 60%, 70%, 80%, 90%, 95%, 96%, 97%, 98%, 99%, or greater change in biological activity. Examples of biological activity for some of the polypeptides of the invention are described below.

By “angiogenesis” is meant the formation of new blood vessels and/or the increase in the volume, diameter, length, or permeability of existing blood vessels, such as blood vessels in a tumor or between a tumor and surrounding tissue. Angiogenesis is associated with a variety of neoplastic and non-neoplastic disorders.

By “angiogenic disorder” is meant a disease associated with excessive or insufficient blood vessel growth, an abnormal blood vessel network, and/or abnormal blood vessel remodeling. For example, insufficient vascular growth can lead to decreased levels of oxygen and nutrients, which are required for cell survival. Angiogenesis, in addition to being critical in metastases formation, also contributes to tumor growth. For any tumors, primary and metastatic, to grow beyond a few millimeters in diameter requires angiogenesis.

By “antisense nucleobase oligomer” or “antisense” is meant a nucleobase oligomer, regardless of length, that is complementary to at least a portion of the coding strand or mRNA of a nucleic acid of the invention (e.g., Tie-1, Tie-1 endodomain, thrombin, VEGFR2 or VEGFR2 endodomain, and EphA2). By a “nucleobase oligomer” is meant a compound that includes a chain of at least eight nucleobases, preferably at least twelve, and most preferably at least sixteen bases, joined together by linkage groups. Included in this definition are natural and non-natural oligonucleotides, both modified and unmodified, as well as oligonucleotide mimetics such as protein Nucleic Acids, locked nucleic acids, and arabinonucleic acids. Numerous nucleobases and linkage groups may be employed in the nucleobase oligomers of the invention, including those described in U.S. Patent Publication Nos. 20030114412 (see for example paragraphs 27-45 of the publication) and 20030114407 (see for example paragraphs 35-52 of the publication), incorporated herein by reference. The nucleobase oligomer can also be targeted to the translational start and stop sites or splicing sequence within the target mRNA. Preferably the antisense nucleobase oligomer comprises from about 8 to 30 nucleotides. The antisense nucleobase oligomer can also contain at least 40, 60, 85, 120, or more consecutive nucleotides that are complementary to the mRNA or DNA target sequence (e.g., Tie-1, Tie-2, tissue factor, thrombin, IP-10, G-CSF, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, KDR, VEGFR2 endodomain, c-RET, MER, and EphA2), and may be as long as the full-length mRNA or gene. Desirably, the antisense nucleobase oligomer contains 8 to 30 nucleotides or more that are complementary to the mRNA or DNA sequence of Tie-1, Tie-2, thrombin, tissue factor, EphA2, KDR, or the VEGFR2 endodomain. Examples of nucleobase oligomers are morpholino oligonucleotides, which have bases similar to natural nucleic acids, but are bound to morpholine rings instead of deoxyribose rings and are linked through phosphorodiamidate groups instead of phosphates. Morpholino oligonucleotides can be designed to any sequence of a target mRNA sequence (e.g., translation start site, an intron sequence, an exon sequence, or a splicing site). Morpholino oligonucleotides can be designed to target the mRNA sequences of any of the target nucleic acids described herein.

By “compound” is meant any small molecule chemical compound, antibody, nucleic acid molecule, or polypeptide, or fragments thereof.

By “decrease” is meant to reduce, preferably by at least 20%, more preferably by at least 30%, and most preferably by at least 40%, 50%, 60%, 70%, 75%, 80%, 85%, 90%, 95%, or more. Decrease can refer, for example, to the symptoms of the disorder being treated or to the levels or biological activity of a polypeptide or nucleic acid of the invention.

By “effective amount” is meant an amount sufficient to treat or prevent a disease of the invention. In one example, the amount is sufficient to treat or prevent a vascular inflammatory disorder. It will be appreciated that there will be many ways known in the art to determine the therapeutic amount for a given application. For example, the pharmacological methods for dosage determination may be used in the therapeutic context.

By “endothelial cell dysfunction” is meant the inability of an endothelial cell to maintain its normal function. Non-limiting examples of endothelial cell function include maintaining balanced vascular tone, inhibiting thrombosis, inhibiting pro-inflammatory processes, maintaining vascular integrity (e.g., non-leakiness of the vasculature), and maintaining an anti-proliferative state in both the endothelium and the surrounding smooth muscle cells. The endothelial cell functions ensure proper vascular pressure, patency, and perfusion. An endothelial cell disorder is any disorder that is characterized by endothelial cell dysfunction. Non-limiting examples of diseases or disorders that are characterized by endothelial cell dysfunction include angiogenic disorders such as cancers which require neovascularization to support tumor growth, infectious diseases, autoimmune disorders, vascular malformations, DiGeorge syndrome, HHT, cavernous hemangioma, transplant arteriopathy, vascular access stenosis associated with hemodialysis, vasculitis, vasculitidis, vascular inflammatory disorders, atherosclerosis, obesity, psoriasis, warts, allergic dermatitis, scar keloids, pyogenic granulomas, blistering disease, Kaposi sarcoma, persistent hyperplastic vitreous syndrome, retinopathy of prematurity, choroidal neovascularization, macular degeneration, diabetic retinopathy, ocular neovascularization, primary pulmonary hypertension, asthma, nasal polyps, inflammatory bowel and periodontal disease, ascites, peritoneal adhesions, contraception, endometriosis, uterine bleeding, ovarian cysts, ovarian hyperstimulation, arthritis, rheumatoid arthritis, chronic articular rheumatism, synovitis, osteoarthritis, osteomyelitis, osteophyte formation, sepsis, and vascular leak. Endothelial cell dysfunction can be determined using assays known in the art including detecting the increased expression of endothelial adhesion molecules or decreased expression or biological activity of nitric oxide synthase (eNOS).

By “EphA2” is meant a polypeptide, or a nucleic acid sequence that encodes it, or fragments or derivatives thereof, that is substantially identical or homologous to or encodes any protein substantially identical to the amino acid set forth in GenBank Accession Numbers NP_{—}004422 (human) and NP_{—}034269 (mouse) and that has EphA2 biological activity. (FIGS. 46 and 47 (human EphA2) and SEQ ID NOs: 9 and 10). EphA2 can also include fragments, derivatives, homologs, orthologues, or analogs of EphA2 that retain at least 25%, 30%, 40%, 50%, 60%, 70%, 80%, 90%, 95%, 96%, 97%, 98%, 99%, or more EphA2 biological activity. EphA2 is a member of the Ephrin receptor family, is membrane bound and has a single kinase domain, an extracellular Cys-rich domain, and two fibronectin type III repeats. EphA2 polypeptides may be isolated from a variety of sources, such as from mammalian tissue, plasma, or cells (e.g., endothelial cells such as HUVEC cells) or from another source, or prepared by recombinant or synthetic methods. The term “EphA2” also encompasses modifications to the polypeptide, fragments, derivatives, analogs, and variants of the EphA2 polypeptide having EphA2 biological activity.

By “EphA2 biological activity” is meant the any of the following activities: pro-inflammatory activity; ligand binding (non-limiting examples of ligands include thrombin and Ephrin A1); kinase activity including but not limited to Ephrin A1 dependent and independent kinase activity; induction of Src dependent and independent kinase activity, wherein the phosphorylation can be autophosphorylation or phosphorylation of another substrate such as other Eph proteins; interaction with other proteins such as Src, FAK, and SH2 domain containing proteins (e.g., CkrL, PI3K (both α and β subunits) and SHP-2); changes in localization; activation or elevation of signaling pathways such the Ras-MAPK and Rho GTP-ase signaling pathways; and modulation of ICAM-1 activation. EphA2 is also thought to play a role in postnatal vascular function and in tumorigenesis.

By “expression” is meant the detection of a nucleic acid molecule or polypeptide by standard art known methods. For example, polypeptide expression is often detected by Western blotting, DNA expression is often detected by Southern blotting or polymerase chain reaction (PCR), and RNA expression is often detected by Northern blotting, PCR, or RNAse protection assays.

By “extended release” is meant formulation of a therapeutic compound such that the release of the active agent (i.e., therapeutic compound), when in combination with another non-active substance (e.g., binder, filler, protein, or polymer), into a physiological buffer (e.g., water or phosphate buffered saline) is decreased relative to the agent's rate of diffusion through a physiological buffer when the agent is not formulated with a non-active substance. Extended release formulations may decrease the rate of release of a therapeutic compound by at least 10%, 20%, 30%, 40%, 50%, 60%, 70%, 80%, 90%, or 100% compared to the rate of release of a therapeutic compound formulation which does not contain a non-active substance (e.g., binder, filler, protein, or polymer).

By “fragment” is meant a portion of a polypeptide or nucleic acid molecule that contains, preferably, at least 10%, 20%, 30%, 40%, 50%, 60%, 70%, 80%, 90%, 95%, or more of the entire length of the reference nucleic acid molecule or polypeptide. A fragment may contain 10, 20, 30, 40, 50, 60, 70, 80, 90, or 100, 200, 300, 400, 500, 600, or more up to 3417 nucleotides for Tie-1, 1170 nucleotides for Tie-1 endodomain, up to 4071 for VEGFR2, up to 1500, 1900, 1971, 2200, or 2271 nucleotides for VEGFR2 endodomain, and up to 2931 nucleotides for EphA2. For polypeptides, a fragment may contain 10, 20, 30, 40, 50, 60, 70, 80, 90, 100, 110, 120, 130, 140, 150, 160, 170, 180, 190, 200, or more up to up to 1139 amino acids for Tie-1, 390 amino acids for Tie-1 endodomain, up to 1356 amino acids for VEGFR2, up to 500, 633, 657, 700, 733, or 757 amino acids for VEGFR2 endodomain, and up to 977 amino acids for EphA2. Preferred fragments include, for example, the Tie-1 endodomain sequence and the VEGFR2 endodomain sequence described herein.

By “heterologous” is meant any two or more nucleic acid or polypeptide sequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous polypeptide will often refer to two or more subsequences that are not found in the same relationship to each other in nature (e.g., a fusion protein).

By “homologous” is meant any gene or polypeptide sequence that bears at least 30% identity, more preferably at least 40%, 50%, 60%, 70%, 80%, and most preferably at least 90%, 95%, 96%, 97%, 98%, 99%, or more identity to a known gene or polypeptide sequence over the length of the comparison sequence. A “homologous” polypeptide can also have at least one biological activity of the comparison polypeptide. For polypeptides, the length of comparison sequences will generally be at least 16 amino acids, preferably at least 20 amino acids, more preferably at least 30, 40, 50, 60, 70, 80, 90, 100, 110, 120, 130, 140, 150, 160, 170, 180, 190, 200, or more amino acids up to up to 1139 amino acids for Tie-1, 390 amino acids for Tie-1 endodomain, up to 1356 amino acids for VEGFR2, up to 500, 633, 657, 700, 733, or 757 amino acids for VEGFR2 endodomain, and up to 977 amino acids for EphA2. For nucleic acids, the length of comparison sequences will generally be at least 50 nucleotides, preferably at least contain 10, 20, 30, 40, 50, 60, 70, 80, 90, or at least 100, 200, 300, 400, 500, 600, 600, or more nucleotides up to 3417 nucleotides for Tie-1, 1170 nucleotides for Tie-1 endodomain, up to 4071 for VEGFR2, up to 1500, 1900, 1971, 2200, or 2271 nucleotides for VEGFR2 endodomain, and up to 2931 nucleotides for EphA2. “Homology” can also refer to a substantial similarity between an epitope used to generate antibodies and the protein or fragment thereof to which the antibodies are directed. In this case, homology refers to a similarity sufficient to elicit the production of antibodies that can specifically recognize the protein or polypeptide.

By “increase” is meant to augment, preferably by at least 20%, more preferably by at least 50%, and most preferably by at least 70%, 75%, 80%, 85%, 90%, 95%, or more. Increase can refer, for example, to the levels or biological activity of a polypeptide or nucleic acid of the invention.

By “inhibitor compound” is meant any small molecule chemical compound (peptidyl or non-peptidyl), antibody, nucleic acid molecule, polypeptide, or fragments thereof that reduces or inhibits the expression levels or biological activity of a protein or nucleic acid molecule by at least 10%, 20%, 30%, 40%, 50%, 60%, 70%, 80%, 90% or more. Non-limiting examples of inhibitor compounds include dominant negative fragments or mutant polypeptides that block the biological activity of the wild type protein; peptidyl or non-peptidyl compounds (e.g., antibodies or antigen-binding fragments thereof) that bind to a protein, for example at a functional domain or substrate binding domain; antisense nucleobase oligomers; morpholinos; double-stranded RNA for RNA interference; small molecule inhibitors; compounds that decrease the half-life of an mRNA or protein; and compounds that decrease transcription or translation of a polypeptide.

By “kinase activity” is meant the ability to catalyze the transfer a phosphate group from adenosine triphosphate (ATP) to a residue (e.g., tyrosine, threonine, or serine) on a substrate polypeptide or protein.

By “metric” is meant a measure. A metric may be used, for example, to compare the levels of a polypeptide or nucleic acid molecule of interest. Exemplary metrics include, but are not limited to, mathematical formulas or algorithms, such as ratios. The metric to be used is that which best discriminates between levels of a polypeptide of the invention polypeptide in a subject having a vascular inflammatory disorder, such as atherosclerosis, or a risk of developing a vascular inflammatory disorder and a normal reference subject. Non-limiting examples of polypeptides that can be included in the metric are Tie-1, thrombin, tissue factor, the VEGFR2 endodomain, ICAM-1, VCAM-1, IL-6, GCSF, CCL20, CCL2, CXCL5, soluble (alternatively spliced) CD44, E-selectins, p38 MAP kinase, eNOS, EGFR, insulin receptor, IGF-IR, AXL, HGFR (c-met), Flt-1, KDR, VEGFR2 endodomain, c-RET, MER, EphA2, and Tie-2. Depending on the metric that is used, the diagnostic indicator of a vascular inflammatory disorder may be significantly above or below a reference value (e.g., from a control subject not having a vascular inflammatory disorders, such as atherosclerosis, or a risk of developing a vascular inflammatory disorder).

By “nitric oxide synthase” or “NOS” is meant an enzyme that catalyzes the formation of nitric oxide (NO) from oxygen and arginine. NOS is a complex enzyme containing several cofactors, a heme group which is part of the catalytic site, an N-terminal oxygenase domain, which belongs to the class of haem-thiolate proteins, and a C-terminal reductase domain which is homologous to NADPH:P450 reductase. NOS produces NO by catalysing a five-electron oxidation of a guanidino nitrogen of L-arginine (L-Arg). Oxidation of L-Arg to L-citrulline occurs via two successive monooxygenation reactions producing N-hydroxy-L-arginine as an intermediate. The interdomain linker between the oxygenase and reductase domains contains a CaM-binding sequence. NO functions at low concentrations as a signal in many diverse physiological processes such as blood pressure control, neurotransmission, learning and memory, and at high concentrations as a defensive cytotoxin. In mammals, three distinct genes encode NOS isozymes: neuronal (nNOS or NOS-1), cytokine-inducible (iNOS or NOS-2) and endothelial (eNOS or NOS-3). eNOS is membrane associated and eNOS localization to endothelial membranes is mediated by cotranslational N-terminal myristoylation and post-translational palmitoylation. Examples of biological activity for eNOS include catalyzing the formation of nitric oxide or “NO” from oxygen and arginine.

In one embodiment, the compound is a compound that increases the phosphorylation of Ser 1177 of eNOS or a compound that increases the dephosphorylation of Thr 495 of eNOS. In another embodiment, the compound is a compound that prevents a reduction in the levels of eNOS or increases the stability of eNOS.

By “pharmaceutically acceptable carrier” is meant a carrier that is physiologically acceptable to the treated mammal while retaining the therapeutic properties of the compound with which it is administered. One exemplary pharmaceutically acceptable carrier substance is physiological saline. Other physiologically acceptable carriers and their formulations are known to one skilled in the art and described, for example, in Remington's Pharmaceutical Sciences, (20^{th} edition), ed. A. Gennaro, 2000, Lippincott, Williams & Wilkins, Philadelphia, Pa.

By “proliferation” is meant an increase in cell number, i.e., by mitosis of the cells. As used herein proliferation does not refer to cancer cell growth.

By “preventing” is meant prophylactic treatment of a subject who is not yet ill, but who is susceptible to, or otherwise at risk of, developing a particular disease. Preferably, a subject is determined to be at risk of developing a vascular inflammatory disorder. “Preventing” can refer to the preclusion of a vascular inflammatory disorder in a patient, desirably a patient that is identified as being at risk for developing a vascular inflammatory disorder. For example, the preventive measures are used to prevent a vascular inflammatory disorder in a patient who has a family history of vascular inflammatory disorders or who has symptoms suggestive of a risk of developing a vascular inflammatory disorder such as stable and unstable angina and claudication. Additional systemic risk factors for vascular inflammatory disorders include hypertension, smoking, hyperlipidemia, and diabetes mellitus, among others. “Preventing” can also refer to the preclusion of the worsening of the symptoms of a vascular inflammatory disorder.

By “protein,” “polypeptide,” or “polypeptide fragment” is meant any chain of more than two amino acids, regardless of post-translational modification (e.g., glycosylation or phosphorylation), constituting all or part of a naturally occurring polypeptide or peptide, or constituting a non-naturally occurring polypeptide or peptide.

By “reduce or inhibit” is meant the ability to cause an overall decrease preferably of 20% or greater, more preferably of 50% or greater, and most preferably of 65%, 70%, 75%, 80%, 85%, 90%, 95%, or greater. Reduce or inhibit can refer to the symptoms of the vascular inflammatory disorder being treated, the biological activity of a polypeptide or nucleic acid of the invention; or the levels of a polypeptide or nucleic acid of the invention. For diagnostic or monitoring applications, reduce or inhibit can refer to the level of protein or nucleic acid, detected by the aforementioned assays (see “expression”).

By “reference sample” is meant any sample, standard, standard curve, or level that is used for comparison purposes. A “normal reference sample” can be, for example, a prior sample taken from the same subject; a normal healthy subject; a sample from a subject not having a vascular inflammatory disorder; a subject that is diagnosed with a propensity to develop a vascular inflammatory disorder but does not yet show symptoms of the disorder; a subject that has been treated for a vascular inflammatory disorder; or a sample of a purified reference polypeptide or nucleic acid molecule of the invention (e.g., Tie-1, Tie-2, tissue factor, thrombin, IP-10, G-CSF, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, KDR, VEGFR2 endodomain, c-RET, MER, and EphA2) at a known normal concentration. By “reference standard or level” is meant a value or number derived from a reference sample. A normal reference standard or level can be a value or number derived from a normal subject who does not have a vascular inflammatory disorder. In preferred embodiments, the reference sample, standard, or level is matched to the sample subject by at least one of the following criteria: age, weight, body mass index (BMI), disease stage, and overall health. A standard curve of levels of purified protein within the normal reference range can also be used as a reference.

By “positive reference” is meant a biological sample, for example, a biological fluid (e.g., urine, blood, serum, plasma, or cerebrospinal fluid), tissue (e.g., vascular tissue or endothelial tissue), or cell (e.g., a vascular endothelial cell), collected from a subject who has a vascular inflammatory disorder (e.g., atherosclerosis) or a propensity to develop a vascular inflammatory disorder (e.g., atherosclerosis) or endothelial cell disorder. In addition, a positive reference may be derived from a subject that is known to have a vascular inflammatory disorder or endothelial cell disorder, that is matched to the sample subject by at least one of the following criteria: age, weight, BMI, disease stage, overall health, prior diagnosis of a vascular inflammatory disorder or endothelial cell disorder, and a family history of a vascular inflammatory disorder or endothelial cell disorder. A positive reference as used herein may also be a purified polypeptide or nucleic acid of the invention (e.g., recombinant or non-recombinant Tie-1, Tie-2, tissue factor, thrombin, IP-10, G-CSF, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, KDR, VEGFR2 endodomain, c-RET, MER, and EphA2), a purified antibody or antigen binding fragment thereof that binds a polypeptide of the invention (e.g., Tie-1, Tie-2, tissue factor, thrombin, IP-10, G-CSF, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, KDR, VEGFR2 endodomain, c-RET, MER, and EphA2), or any biological sample (e.g., a biological fluid, tissue, or cell) that contains a polypeptide or nucleic acid of the invention or an antibody that specifically binds to a polypeptide of the invention. A standard curve of levels of purified protein, nucleic acid, or antibody for any of the polypeptides of the invention (e.g., Tie-1, Tie-2, tissue factor, thrombin, IP-10, G-CSF, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, KDR, VEGFR2 endodomain, c-RET, MER, and EphA2) within a positive reference range can also be used as a reference.

By “sample” is meant a bodily fluid (e.g., urine, blood, serum, plasma, or cerebrospinal fluid), tissue (e.g., cardiac tissue or endothelial tissue), or cell (e.g., endothelial cell) in which a polypeptide or nucleic acid molecule of the invention is normally detectable.

By “small RNA” is meant any RNA molecule, either single-stranded or double-stranded” that is at least 15 nucleotides, preferably, 17, 18, 19, 20, 21, 22, 23, 24, 25, 26, 27, 28, 29, 30, 31, 32, 33, 34, or 35, nucleotides in length and even up to 50 or 100 nucleotides in length (inclusive of all integers in between). Preferably, the small RNA is capable of mediating RNAi. As used herein the phrase “mediates RNAi” refers to the ability to distinguish which RNAs are to be degraded by the RNAi machinery or process. Included within the term small RNA are “small interfering RNAs” and “microRNA.” In general, microRNAs (miRNAs) are small (e.g., 17-26 nucleotides), single-stranded noncoding RNAs that are processed from approximately 70 nucleotide hairpin precursor RNAs by Dicer. Small interfering RNAs (siRNAs) are of a similar size and are also non-coding, however, siRNAs are processed from long dsRNAs and are usually double stranded. siRNAs can also include short hairpin RNAs in which both strands of an siRNA duplex are included within a single RNA molecule. Small RNAs can be used to describe both types of RNA. These terms include double-stranded RNA, single-stranded RNA, isolated RNA (partially purified RNA, essentially pure RNA, synthetic RNA, recombinantly produced RNA), as well as altered RNA that differs from naturally occurring RNA by the addition, deletion, substitution and/or alteration of one or more nucleotides. Such alterations can include addition of non-nucleotide material, such as to the end(s) of the small RNA or internally (at one or more nucleotides of the RNA). Nucleotides in the RNA molecules of the present invention can also comprise non-standard nucleotides, including non-naturally occurring nucleotides or deoxyribonucleotides. In a preferred embodiment, the RNA molecules contain a 3′ hydroxyl group.

By “specifically binds” is meant a compound or antibody which recognizes and binds a polypeptide of the invention but that does not substantially recognize and bind other molecules in a sample, for example, a biological sample, which naturally includes a polypeptide of the invention. In one example, an antibody that specifically binds a VEGFR2 endodomain does not bind to VEGFR2.

By “subject” is meant a mammal, including, but not limited to, a human or non-human mammal, such as a bovine, equine, canine, ovine, or feline.

By “substantially identical” is meant a nucleic acid or amino acid sequence that, when optimally aligned, for example using the methods described below, share at least 60%, 65%, 70%, 75%, 80%, 85%, 90%, 95%, 96%, 97%, 98%, 99%, or 100% sequence identity with a second nucleic acid or amino acid sequence, e.g., a Tie-1, Tie-1 endodomain, EphA2, VEGFR2 or VEGFR2 endodomain sequence. “Substantial identity” may be used to refer to various types and lengths of sequence, such as full-length sequence, epitopes or immunogenic peptides, functional domains, coding and/or regulatory sequences, exons, introns, promoters, and genomic sequences. Percent identity between two polypeptides or nucleic acid sequences is determined in various ways that are within the skill in the art, for instance, using publicly available computer software such as Smith Waterman Alignment (Smith and Waterman, J. Mol. Biol. 147:195-7, 1981); “BestFit” (Smith and Waterman, Advances in Applied Mathematics, 482-489, 1981) as incorporated into GeneMatcher Plus™, Schwarz and Dayhof, “Atlas of Protein Sequence and Structure,” Dayhof, M. O., Ed pp 353-358, 1979; BLAST program (Basic Local Alignment Search Tool; (Altschul, S. F., W. Gish, et al., J. Mol. Biol. 215: 403-410, 1990), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins, the length of comparison sequences will be at least 10 amino acids, preferably 20, 30, 40, 50, 60, 70, 80, 90, 100, 110, 120, 130, 140, 150, 160, 170, 180, 190, 200, 209 amino acids or more. For nucleic acids, the length of comparison sequences will generally be at least 10, 20, 30, 40, 50, 60, 70, 80, 90, or 100, 200, 300, 400, 500, 600, 627, or more nucleotides. It is understood that for the purposes of determining sequence identity when comparing a DNA sequence to an RNA sequence, a thymine nucleotide is equivalent to a uracil nucleotide. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By “thrombin” is meant a polypeptide, or a nucleic acid sequence that encodes it, or fragments or derivatives thereof, that is substantially identical or homologous to or encodes any protein substantially identical to the amino acid set forth in GenBank Accession Numbers NP_{—}000497 (human) and NP_{—}034298 (mouse) and that has thrombin biological activity. (See FIGS. 44 and 45 and SEQ ID NOs: 7 and 8). Thrombin can also include fragments, derivatives, homologs, orthologues, or analogs of thrombin that retain at least 25%, 30%, 40%, 50%, 60%, 70%, 80%, 90%, 95%, 96%, 97%, 98%, 99%, or more thrombin biological activity. The thrombin polypeptides may be isolated from a variety of sources, such as from mammalian tissue, plasma, or cells (e.g., endothelial cells such as HUVEC cells) or from another source, or prepared by recombinant or synthetic methods. The term “thrombin” also encompasses modifications to the polypeptide, fragments, derivatives, analogs, and variants of the thrombin polypeptide having thrombin biological activity.

By “thrombin biological activity” is meant the any of the following procoagulant activities: cleavage of thrombin dependent substrate such as fibrinogen, activation of substrates such as factors XI, V, VIII, and protein C; proteolytic activity (e.g., conversion of fibrinogen to fibrin); ligand binding, receptor binding and activation (e.g., protease-activated receptors (PAR) such as PAR 1 and PAR3) platelet activation and aggregation in many settings, such arterial thrombosis or subacute thrombosis; and/or any of the following pro-inflammatory activities described herein including, upregulation of ICAM-1, thrombin-mediated increased leukocyte attachment to thrombin stimulated cells, intracellular gap formation and endothelial cell permeability, and induction of an increase in the level or biological activities of tyrosine kinase receptor proteins including but not limited to EGFR, insulin receptor, IGF-IR, AXL, HGFR (c-met), Flt-1, KDR, VEGFR2 endodomain, c-RET, MER, EphA2, Tie-1, and Tie-2).

By “thrombin inhibitor” is meant any compound which inhibits the biological activity of thrombin known in the art or described herein. A thrombin inhibitor may inhibit the catalytic conversion of fibrinogen to fibrin, activation of Factor V to Va, Factor VIII to VIIIa, Factor XIII to XIIIa, and activation of platelets, or any of the pro-inflammatory activities of thrombin described herein. Compounds may be identified as thrombin inhibitors by evaluating the compounds in assays described in S. D. Lewis et al., Thrombosis Research 70 pp. 173-190 (1993). Additional exemplary thrombin inhibitors are described in U.S. Pat. No. 6,232,315. One assay involves the measurement of rates of substrate hydrolysis, and the other involves measurement of activated partial thromboplastin time. Assays for the biological activity of thrombin are also described herein. In one example a thrombin inhibitor will reduce or inhibit leukocyte attachment to an endothelial cell, reduce or inhibit thrombin mediated ICAM-1 upregulation, and reduce or inhibit endothelial cell permeability or intracellular gap formation.

By “Tie-1” is meant a polypeptide, or a nucleic acid sequence that encodes it, or fragments or derivatives thereof, that is substantially identical or homologous to or encodes any protein substantially identical to the amino acid set forth in GenBank Accession Numbers P35590 (human), NP_{—}035717 (mouse), and CAA50556 (mouse), and that has Tie-1 biological activity. (See FIGS. 40 and 41 and SEQ ID NOs: 3 and 4 for the human Tie-1 sequences.) Tie-1 can also include fragments, derivatives, homologs, orthologs, or analogs of Tie-1 that retain at least 25%, 30%, 40%, 50%, 60%, 70%, 80%, 90%, 95%, 96%, 97%, 98%, 99%, or more Tie-1 biological activity. The Tie-1 polypeptides may be isolated from a variety of sources, such as from mammalian tissue or cells (e.g., endothelial cells such as HUVEC cells) or from another source, or prepared by recombinant or synthetic methods. The term “Tie-1” also encompasses modifications to the polypeptide, fragments, derivatives, analogs, and variants of the Tie-1 polypeptide having Tie-1 biological activity.

By “Tie-1 endodomain” is meant a polypeptide, or a nucleic acid sequence that encodes it, or fragments or derivatives thereof, that is a biologically active fragment of Tie-1 (see FIGS. 42 and 43). Generally, the Tie-1 endodomain sequence includes the sequence of SEQ ID NO: 5 (amino acid) or SEQ ID NO: 6 (nucleotide). The term “Tie-1 endodomain” also encompasses modifications to the polypeptide, fragments, derivatives, homologs, orthologs, analogs, and variants of the Tie-1 endodomain polypeptide having Tie-1 biological activity. Exemplary homologs of Tie-1 endodomain include the zebrafish Tie-1 endodomain which has a high protein sequence identity to human (>87%) and a low GC content in the coding sequence (−46%) and the mouse Tie-1 endodomain which has a high protein sequence identity to human (>96%) and a low GC content in the coding sequence (−57%). In vitro experiments have shown that Tie-1 undergoes ectodomain shedding upon stimulation to generate a membrane-bound C-terminal endodomain. External stimuli that can result in Tie-1 cleavage include phorbol ester, VEGF, thrombin, TNFα, LPS (Yabkowitz, Meyer et al., Blood 90: 706-715, (1997); Yabkowitz, Meyer et al., Blood 93: 1969-1979, (1999)) and changes in sheer stress (Chen-Konak, Guetta-Shubin et al., Faseb J 17: 2121-2123, (2003)). This shedding event appears to be dependent on a cell-surface bound metalloproteinase (McCarthy, Burrows et al., Lab Invest 79: 889-895, (1999); Yabkowitz, Meyer et al., Blood 93: 1969-1979, (1999)). Prior to the discoveries described herein, the phosphorylation status or kinase activity of the Tie-1 endodomain had not been described.

By “Tie-1 biological activity” is meant any of the following activities: cleavage of the Tie-1 ectodomain to produce the activated Tie-1 endodomain; ligand binding; ATP binding; kinase activity; activation (increased expression or biological activity) of cytokine or adhesion markers, such as ICAM-1, VCAM-1, IL-6, GCSF, IL-10, CCL20, CCL2, CXCL5, soluble (alternatively spliced) CD44, and E-selectins; inhibition or downregulation of eNOS expression or biological activity; activation (increased expression or biological activity) of thrombin, tissue factor, or p38 MAP kinase; promotion of endothelial cell adhesion; and promotion of smooth muscle cell proliferation or migration. Assays for each of these activities are known in the art and described herein.

By “Tie-1 inhibitor compound” is meant any small molecule chemical compound (peptidyl or non-peptidyl), antibody, nucleic acid molecule, polypeptide, or fragments thereof that reduces or inhibits the expression levels or biological activity of Tie-1 by at least 10%, 20%, 30%, 40%, 50%, 60%, 70%, 80%, 90% or more. Non-limiting examples of Tie-1 inhibitor compounds include fragments of Tie-1 (e.g., dominant negative fragments or Tie-1 fragments that are unable to bind ATP or to undergo cleavage of the ectodomain); peptidyl or non-peptidyl compounds that specifically bind Tie-1 (e.g., antibodies or antigen-binding fragments thereof), for example at the ATP binding domain or substrate binding domain of Tie-1; peptidyl or non-peptidyl compounds that block cleavage of the Tie-1 ectodomain or shedding of the ectodomainl antisense nucleobase oligomers; morpholinos directed to Tie-1; double-stranded RNA directed to Tie-1 for RNA interference; small molecule inhibitors; compounds that decrease the half-life of Tie-1 mRNA or protein; compounds that decrease transcription or translation of Tie-1; and compounds that block Tie-1-kinase activity (e.g., by binding to the ATP binding pocket or additional regions of the protein required for kinase activity). In addition, a Tie-1 inhibitor compound can be a compound that inhibits Tie-2 kinase activity, for example by binding to the ATP binding pocket, which is highly conserved between Tie-1 and Tie-2. Tie-1 inhibitor compounds can be identified using the compound in any of the assays described above for Tie-1 biological activity and identifying a compound that shows at least a 10%, 20%, 30%, 40%, 50%, 60%, 70%, 80%, 90% or more decrease in Tie-1 activity as compared to a control where the compound has not been added.

By “treating” is meant administering a compound or a pharmaceutical composition for prophylactic and/or therapeutic purposes or administering treatment to a subject already suffering from a disease to improve the subject's condition or to a subject who is at risk of developing a disease. As it pertains to vascular inflammatory disorders, treating can include improving or ameliorating the symptoms of a vascular inflammatory disorder and prophylactic treatment can include preventing the progression of a mild vascular inflammatory disorder to a more serious form. Prophylactic treatment can be monitored, for e.g., by performing angiography of the coronary and lower extremity vasculature and pharmacologic and exercise cardiovascular stress tests, measurement of flow rate and vascular ultrasound. Treating may also mean to prevent the onset of a vascular inflammatory disorder in a patient identified as at risk for developing a vascular inflammatory disorder (e.g., using any diagnostic method known in the art or the diagnostic methods described herein).

By “vascular inflammatory disorder” is meant any disorder of the vasculature that includes one or more of the following characteristics: endothelial cell dysfunction, increased angiogenesis, calcification, increased smooth muscle cell proliferation, increased attachment of leukocytes, and increased infiltration of leukocytes such as monocytes, T cells, and foamy macrophages. Preferably, the vascular inflammatory disorder includes at least two, at least three, or at least four or more of the above characteristics. Endothelial cell dysfunction is determined using assays known in the art including detecting the increased expression of endothelial adhesion molecules or decreased expression or biological activity of nitric oxide synthase (eNOS). Angiogenesis is measured using a variety of angiogenesis assays known in the art including the detection of pro-angiogenic markers, such as VEGF or VEGF receptors. Smooth muscle (SM) cell proliferation is measured by the increased presence of smooth muscle cells or SM-like cells identified by markers such as smooth muscle cell actin and desmin. Examples of vascular inflammatory disorders include arteriosclerosis (acute or chronic), atherosclerosis (acute or chronic), neointimal hyperplasia (e.g., venous neointimal hyperplasia, peripheral vascular disease, and dialysis vascular access), sepsis, vascular leak, and rheumatoid arthritis. It should be noted that due to the overlap between vascular inflammatory disorders and endothelial cell dysfunction, many of the disorders fall into both categories.

By “vector” is meant a DNA molecule, usually derived from a plasmid or bacteriophage, into which fragments of DNA may be inserted or cloned. A recombinant vector will contain one or more unique restriction sites, and may be capable of autonomous replication in a defined host or vehicle organism such that the cloned sequence is reproducible. A vector contains a promoter operably linked to a gene or coding region such that, upon transfection into a recipient cell, an RNA is expressed.

By “VEGF receptor 2” or “VEGFR2” (also known as KDR) is meant the kinase insert domain-containing receptor (see, for example, WO 92/14748; Matthews et al., Proc. Natl. Acad. Sci. USA, 88: 9026 (1991); Terman et al., Biochem. Biophys. Res. Comm., 187: 1579 (1992); WO 94/11499), which belongs to the receptor type tyrosine kinase family. (See FIGS. 23A and 23B; SEQ ID NOs: 1 and 2). KDR is a membrane protein of 180 to 200 kilodalton in molecular weight which has an extracellular domain consisting of 7 immunoglobulin-like (Ig-like) regions and an intracellular domain consisting of a tyrosine kinase region. “Immunoglobulin-like domain” or “Ig-like domain” refers to each of the seven independent and distinct domains that are found in the extracellular ligand-binding region of the flt-1, KDR and FLT4 receptors. Ig-like domains are generally referred to by number, the number designating the specific domain as it is shown in FIG. 1 of U.S. Pat. No. 5,952,199, herein incorporated by reference. As used herein, the term “Ig-like domain” is intended to encompass not only the complete wild-type domain, but also insertion, deletion, and substitution variants thereof which substantially retain the functional characteristics of the intact domain. It will be readily apparent to those of ordinary skill in the art that numerous variants of the Ig-like domains of the KDR receptor can be obtained which will retain substantially the same functional characteristics as the wild type domain.

It has been reported that VEGF specifically binds to KDR at Kd values of 75 pM and that KDR is expressed in vascular endothelial cells in a specific manner (Quinn et al., Proc. Natl. Acad. Sci. USA, 90: 7533 (1993); Peters et al., Proc. Natl. Acad. Sci. USA, 90: 8915 (1993)). The term “VEGFR2 receptor” as used herein is meant to encompass not only the KDR receptor but also the murine homologue of the human KDR receptor, designated FLK-1.

By “VEGFR2 endodomain” is meant a polypeptide, or a nucleic acid sequence that encodes it, or fragments or derivatives thereof, that is approximately 120 kDa (but can be 90 kDa, 100 kDa, 105 kDa, 110 kDa, 115 kDa, 120 kDa, 125 kDa, 130 kDa, 135 kDa, 140 kDa, 145 kDa, and 150 kDa depending on the conditions used for determining the molecular weight) and is substantially identical or homologous to at least a portion of the carboxy-terminus of VEGFR2 and can be detected using an antibody that binds to the carboxy-terminus of VEGFR2 but is not the full length VEGFR2. Examples of antibodies that are directed to the carboxy-terminus of VEGFR2 include anti-phospho KDR (Y1054/Y1059) from Abcam (Catalog number 5473-50), anti-phospho KDR (Y951) from Cell Signaling (Catalog number 3221), and anti KDR antibody from Santa Cruz Biotechnology (Catalog number SC6251). In one embodiment, the VEGFR2 endodomain includes a sequence that is at least 80%, 85%, 90%, 95%, or 99% or more identical to amino acids 700 to 1200, 700 to 1356, or 600 to 1356 of the sequence set forth in SEQ ID NO: 1. For nucleic acid molecules encoding the VEGFR2 endodomain, the nucleic acid sequence can, for example, include a sequence that is at least 80%, 85%, 90%, 95%, or 99% or more identical to nucleotides 2100 to 3600, 2100 to 4071, or 1800 to 4071 of SEQ ID NO: 2.

Thrombin has both pro-inflammatory effects and procoagulant effects and methods for specifically inhibiting the pro-inflammatory effects without affecting the procoagulant effects would be extremely useful for the treatment of vascular inflammatory disorders. We have discovered polypeptides, including Tie-1, Tie-2, tissue factor, thrombin, IP-10, G-CSF, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, KDR, c-RET, MER, and EphA2 and fragments thereof, that are specifically involved in regulation of the pro-inflammatory effects of thrombin on endothelial cells and that inhibitors of such molecules can be used for the treatment of vascular inflammatory disorders. Furthermore, the specificity of the signaling pathways that we have discovered allows for a specific targeted therapeutic effect on the inflammatory pathways regulated by thrombin in the absence of any effect on the pro-coagulant functions of thrombin. In addition, any one or more of these signaling molecules may act in concert such that the use of a combination of inhibitors targeting one or more of the signaling molecules may produce a synergistic effect for the treatment of a vascular inflammatory disorder or endothelial cell disorder.

Other features and advantages of the invention will be apparent from the following description of the preferred embodiments thereof, and from the claims.

### BRIEF DESCRIPTION OF THE DRAWINGS

The application file contains drawings executed in color (FIGS. 2, 3, 4, 13, 21, 22, and 35). Copies of this patent or patent application with color drawings will be provided by the Office upon request and payment of the necessary fee.

FIGS. 1A-1B show increased cytokine expression by cytokine antibody array in Tie-1 endomain expressing endothelial cells. FIGS. 1A-1B show upregulation of three cytokines: G-CSF, IP-10, and IL-6 (FIG. 1B, open arrows: G-CSF; solid arrows: IP-10; asterisks: IL-6) in Tie-1 infected cells when compared to the GFP-virus infected cells (FIG. 1A).

FIGS. 2A-2I show the proinflammatory response elicited by Tie-1 endodomain expression in endothelial cells. FIG. 2A and FIG. 2B show an upregulation of IP-10 by ELISA and real-time PCR, respectively, in HPAECs that stably express zebrafish Tie-1 endothelium. FIG. 2C and FIG. 2D show upregulation of IP-10 by real-time PCR in endothelial cells HPAEC and HUVEC, respectively, that transiently express mouse Tie-1 endodomain via adenoviral infection. FIG. 2E is a series of photographs that show HUVEC cells infected with adenovirus are unchanged morphologically, which suggest the infected cells remain healthy. FIG. 2Ei and 2Eii are HUVEC cells infected with GFP virus, and FIGS. 2Eiii and 2Eiv are HUVEC cells infected with Tie-1 endodomain virus. FIGS. 2Ei and 2Eiii are phase contrast microscopy images, and FIG. 2E ii and 2Eiv are fluorescent microscopy images. FIGS. 2F, 2G, and 2H are the results of real-time PCR experiments demonstrating that ICAM-1, VCAM-1, and IL-6, respectively, are upregulated in HUVEC cells transiently expressing Tie-1 endodomain via adenoviral infection. FIG. 2I is a western blot showing G-CSF upregulation from transient expression of Tie-1 endodomain in HUVECS in HUVEC conditioned medium.

FIGS. 3A-3C are fluorescent microscopy images that show Tie-1 endodomain expression enhances adhesion of monocytes to HUVECs. FIG. 3A and FIG. 3B are photomicrographs that show HUVEC cells expressing GFP or Tie-1 endodomain respectively, incubated with U937 cells (Red) visualized by fluorescent microscopy. FIG. 3C is a graph summarizing these results. Number of attached cells in the control was arbitrarily set to 1.

FIGS. 4A-4B are fluorescent microscopy images that show HUVECs expressing the Tie-1 endodomain secrete a migratory stimulant for smooth muscle cells. FIG. 4B and FIG. 4A show smooth muscle cells (in red) that migrate and do not respond to conditioned media from HUVECs expressing Tie-1 endodomain or GFP, respectively. Representative results are shown.

FIG. 5 shows basal activation of p38 in endothelial cells is elevated by expression of Tie-1 endodomain. HUVECs were either infected with GFP-alone (lane 1) or Tie-1 endodomain adenovirus (lane 2). Activation of p38 was assessed by western blotting using a phospho-specific antibody.

FIG. 6 shows overexpression of Tie-1 endodomain in HUVECs induces activation of thrombin. HUVECs were either expressing GFP or Tie-1 endodomain. Whole human plasma supplemented with an excess chromogenic thrombin substrate (sarcosine-Pro-Arg-pNA) (Duncan, Bowie et al., Clin Chem 31: 853-855, (1985)) was added to HUVECs. Cleavage carboxyl-terminal to the arginine residue by thrombin releases p-nitrophenol, which can be monitored by absorbance at 405 nm.

FIGS. 7A-7B show stimulation of HUVECs with thrombin triggers transactivation of multiple receptor tyrosine kinases. FIG. 7A is an image of a phospho-RTK antibody array using lysates prepared from HUVEC monolayer (unstimulated, top; thrombin stimulated, bottom) showing activation of multiple RTKs. Arrows point at the second spot of each duplicate immunoprecipitate. FIG. 7B shows lysates from RCC4, a renal cancer carcinoma cell line, in a parallel experiment. Note that only EGFR was significantly transactivated by thrombin.

FIGS. 8A-8B show the transactivation of receptor tyrosine kinases by thrombin treatment was validated by immunoprecipitation/immunoblot experiments. In FIG. 8A increasing concentration of thrombin (lane 0 to lane 5) added to HUVECS induces greater phosphorylation of tyrosine kinases. Tyrosine phosphorylated cellular proteins were immunoprecipitated with an anti phospho-tyrosine antibody (4G10). After SDS-PAGE, phosphorylation status of each RTK was examined by western blotting using a specific antibody. Both VEGFR-2 and Tie-1 undergo proteolytic cleavage to yield a protein of a smaller size (see text). FIG. 8A lane CM* represents an experiment where HUVECs were treated with conditioned medium of HUVECs stimulated with thrombin. FIG. 8B shows thrombin treatment of HUVECs (FIG. 8B, lane 2) induces ectodomain shedding of full-length Tie-1 (solid arrow) and generation of Tie-1 endodomain (open arrow) compared to untreated HUVECS (FIG. 8B, lane 1). Molecular weights are in kDa.

FIGS. 9A-9B. Thrombin stimulation causes proteolytic cleavage of VEGFR-2 to generate a 120-kDa species. In FIG. 9A, VEGFR-2 was immunoprecipitated from lysates prepared from confluent HUVEC monolayer using an anti VEGFR-2 antibody directed at the C-terminus of the protein. After SDS-PAGE, VEGFR-2 was visualized by western blot using the same antibody. Upon thrombin treatment, the intensity of full-length VEGFR-2 band decreased (solid arrow) with a concomitant appearance of a band of ˜120 kDa (open arrow). In FIG. 9B, 4G10 immunoprecipitates were probed with a different antibody directed at a different phosphorylation site (Y951). A specific band with the molecular weight of ˜120 kDa was detected upon thrombin treatment using this antibody confirming that the 120 kDa band was of VEGFR2 origin. (−): No thrombin; (T): 5 U/ml thrombin. Molecular weights are in kDa.

FIG. 10 shows activation of VEGFR-2 by thrombin is an early signaling event. HUVEC monolayer was stimulated with thrombin for the length of time indicated. Lysates were prepared and tyrosine phosphorylated proteins were immunoprecipitated by 4G10. Phosphorylation status of VEGFR-2 was probed using anti phospho VEGFR-2 (Y1054/Y1059) antibody. Molecular weights are in kDa.

FIG. 11 shows the activation of VEGFR is a pre-requisite for transactivation of several receptor tyrosine kinases by thrombin. Confluent HUVECs were pretreated with 10 μM SU5416, a specific VEGFR inhibitor, for 2 hrs, followed by stimulation with thrombin. 4G10 immunoprecipitates were fractioned by SDS-PAGE, and receptor tyrosine kinases were detected by western blot using specific antibodies. (−): No thrombin; (T): 5 U/ml thrombin. Molecular weights are in kDa.

FIG. 12 is a graph showing the thrombin induced-endothelial barrier dysfunction requires VEGFR activity. Confluent HUVEC monolayers were established in Transwell inserts. Cells were pretreated with either DMSO (vehicle) or 10 μM SU5416 for one hour, followed by the addition of fluorescein-labeled BSA and thrombin (5 U/ml). After 10 minutes of stimulation, fluorescein-labeled BSA that had diffused to the lower chamber was detected by fluorescence measurement and used as a surrogate marker of endothelial permeability. T: thrombin; SU: SU5416.

FIG. 13 is a series of photomicrographs showing PAR-1 mediated endothelial gap formation requires the activity of VEGFR. Confluent HUVEC monolayers were grown on collagen-coated glass slides and pre-treated with either DMSO or 10 μM SU5416 for 2 hours, followed by stimulation with thrombin or PAR-1 activating peptide for 15 mins. VE-cadherin, actin stress fiber, and nuclei were stained with anti VE-cadherin antibody (green), phalloidin (red), and DAPI (blue), respectively.

FIGS. 14A-14C show transactivation of VEGFR is critical for tyrosine phosphorylation of VE-cadherin and p120 but not involved in MLC signaling. In FIG. 14A HUVECs were pretreated with either DMSO or 10 μM SU5416, followed by stimulation with thrombin (5 minutes). To preserve tyrosine phosphorylation, cells were then treated with 2 mM Na₃VO₄/2 mM H₂O₂ for 5 mins prior to lysis (Lampugnani, Corada et al., J. Cell Sci. 110 (Pt 17): 2065-2077, (1997)). A portion of the clarified total lysates was analyzed by western blot using an anti-phospho MLC antibody. The membrane was stripped and reblotted with anti GAPDH antibody. In FIG. 14B VE-cadherin was immunoprecipitated from lysates prepared in FIG. 14A. Tyrosine phosphorylation was detected by 4G10 antibody (FIG. 14B, i). The membrane was stripped and reblotted with an anti VE-cadherin antibody (FIG. 14B, ii). In FIG. 14C, HUVECs were pretreated with 10 μM SU5416 for 2 hrs, followed by stimulation with thrombin. 4G10 immunoprecipitates were fractioned by SDS-PAGE, and p120 was detected by western blot. (−): No thrombin; (T): 5 U/ml thrombin. Molecular weights are in kDa.

FIG. 15 is a schematic showing a working model of how Tie-1 induces endothelial inflammation and may be crucial in atherosclerosis development.

FIG. 16 is a schematic showing inducible expression knockdown in mice using shRNAmir. tTA expression is under the CMV promoter. The targeting shRNAmir expression is controlled by TRE. The activity of tTA is suppressed in the presence of doxycycline (Dox). Therefore, the shRNAmir is not transcribed. Upon Dox withdrawal, tTA becomes active and transactivates the expression of the shRNAmir. Thus, doxycycline governs the temporal expression of the shRNAmir. The shRNAmir is first transcribed as an artificial primary shRNAmir and is processed by Drosha into a precursor shRNAmir. It is further processed by Dicer to become the mature shRNAmir (Cullen, Nat Genet. 37: 1163-1165, (2005)). The antisense strand (blue) targets the specific mRNA. Note that tTA can be expressed under the control of an endothelial specific promoter (e.g. Tie-2 promoter). This confers endothelial specific expression of tTA, achieving specific gene expression knockdown only in the endothelium.

FIGS. 17A-17C are schematics showing construction of shRNAmir for Tie-1 knockdown. FIG. 17A is an illustration of the expression vector SIN-TREmiR30-PIG (TMP). The shRNAmir sequence is cloned between the XhoI and EcoRI sites. FIG. 17B shows the design of a Tie-1 shRNAmir (SEQ ID NO: 44). The sequences in red and blue are the sense and antisense strand of the shRNAmir, respectively. The sequence in green is the miR-30 loop structure. The mature shRNAmir will target nucleotides 1015 to 1036 of mouse Tie-1 mRNA. Two more regions of the mRNA will be targeted (see text). FIG. 17C is an illustration of the strategy of fragment creation for mouse construction. The BglII/SphI fragment of the shRNAmir clone is excised from the TMP vector and ligated, together with a SphI/HindIII fragment containing the SV40 polyadenylation signal, into pLITMUS28i. The BglII/HindIII fragment from the resultant clone will contain the following elements: TRE, shRNAmir coding sequence, and an polyadenylation signal. This fragment will be used in transgenic mouse construction.

FIG. 18 shows the PCR strategy used to clone soluble CD44.

FIGS. 19A and 19B show the mRNA sequences of soluble CD44 #1 and #2 (SEQ ID NOs: 35 and 36). FIG. 19C shows a sequence alignment of the amino acid sequences of soluble CD44 #1 and #2 (SEQ ID NOs: 37 and 43) and five known variants of human CD44 (SEQ ID NOs: 38-42).

FIG. 20 is a graph showing that the expression of Tie-1 in HUVEC upregulates CCL20, CXCL5, and E-selectin as assayed by real time PCR analysis.

FIG. 21 is an autoradiograph and a series of photomicrographs showing the suppression of Tie-2 in HUVEC prevents endothelial cells from reforming a continuous monolayer after thrombin stimulation.

FIG. 22 is a series of photomicrographs showing that SU5416 inhibited PAR-1 induced vascular leak in mice. For these experiments, the left and right ears of a mouse were pretreated with 10 μl of DMSO or 10 μM SU5416, respectively, for one hour. Then 20 μl of 5 mM PAR-1 activating peptide and 500 μl 0.1% Evan blue was injected into the mouse by tail vein injection. About 15 minutes later the extent of vascular leaks in the ears was documented by photography.

FIG. 23A shows the amino acid sequence of VEGF receptor 2 (VEGFR2) (SEQ ID NO: 1).

FIG. 23B shows the corresponding cDNA sequence (SEQ ID NO: 2).

FIG. 24A is a graph showing a decrease of eNOS mRNA due to Tie-1 expression using real time PCR analysis. Solid bars: GFP adenovirus infection; open bars: Tie-1 adenovirus infection. eNOS mRNA level in control GFP cells was arbitrarily set to 1. Hours indicated are post infection.

FIG. 24B is a western blot analysis showing eNOS dowregulation at the protein level by Tie-1 expression. Molecular weights in kDa.

FIGS. 25A-B shows Tie-1 overexpressed in endothelial cells is tyrosine phosphorylated. FIG. 25A is a western blot showing overexpressed and endogenous Tie-1 from HUVECs infected with either Tie-1 or GFP adenovirus were immunoprecipitated with a Tie-1 specific antibody. Tyrosine phosphorylation of Tie-1 was determined by western blotting with an anti phosphotyrosine antibody (4G10) (left). The membrane was stripped and reblotted with the Tie-1 antibody (right). FIG. 25B is a western blot showing the reverse experiment, which was performed to show that Tie-1 is tyrosine phosphorylated when overexpressed in HUVECs. The lysates were first immunoprecipitated with 4G10 to capture all tyrosine-phosphorylated proteins. The immunoprecipitates were fractionated by SDS-PAGE and Tie-1 detected by western blotting. Note that at endogenous level, Tie-1 is not tyrosine phosphorylated.

FIGS. 26A-C shows Tie-1 expression upregulates IL-6 in HUVECs. In FIG. 26A, conditioned media from GFP- (right) and Tie-1-(left) adenovirus infected HUVECs were used in an antibody array experiment. Antibodies were spotted in duplicate on the membrane. Boxed dots were positive controls for orientation. IL-6 was upregulated by Tie-1 expression (arrowheads). In FIG. 26B, real-time PCR experiments showing IL-6 mRNA level was increased when Tie-1 was overexpressed. IL-6 mRNA level in GFP-infected cells was arbitrarily set to 1. FIG. 26C is an ELISA showing IL-6 protein upregulated in conditioned medium (48 hrs) from HUVEC by Tie-1 overexpression.

FIG. 27 is a graph showing Tie-1 overexpression in HUVECs upregulates IP-10, ICAM-1, VCAM-1, E-selectin, and CCL2, but not PDGF-B. Expression of genes of interest was determined by real-time PCR using cDNA prepared HUVECs infected with either GFP or Tie-1 adenovirus (48 hrs). mRNA levels in GFP-infected cells were arbitrarily set to 1.

FIG. 28 is a graph showing that Tie-1 induced endothelial inflammation is p38 dependent. Real-time PCR experiments showing that inhibition of p38 with SB-203580 (SB) significantly blocked Tie-1-induced inflammation in HUVECs. mRNA levels in GFP-infected cells were arbitrarily set to 1.

FIGS. 29A-H show Tie-1 induced inflammation is significantly higher in endothelial cells of aortic origin. Real-time PCR showing that upregulation of E-selectin, VCAM-1, and IP-10 was significantly higher in HAECs than in HUVECs (FIGS. 29 A-29C), whereas expression of ICAM-1, CCL2, and IL-6 were similar in both cell types (FIGS. 29D-29F). PDGF was not induced by Tie-1 in either cell type (FIG. 29G). Western blot to show level of Tie-1 expression (FIG. 29H). Open bars: Ad-GFP infection; solid bars: Ad-Tie-1 infection; gray bars: HAECs were infected with half the amount of Tie-1 adenovirus to show that even at this lower Tie-1 expression, E-selectin, VCAM-1, and IP-10 were upregulated more in HACEs than in HUVECs. mRNA levels in GFP-infected cells were arbitrarily set to 1. MW in kDa.

FIGS. 30A-30C show Tie-1 expression promotes attachment of U937 cells to HAECs. U937 attachment to HACEs 48 hrs after infected with GFP-adenovirus (FIG. 30A) or Tie-1 adenovirus (FIG. 30B). FIG. 30C is a series of western blots showing expression of adhesion molecules in HAECs when Tie-1 is overexpressed. T: Tie-1 adenovirus infection; G: GFP adenovirus infection. Note that endogenous Tie-1 was significantly lower than the overexpressed Tie-1 level and thus not detected in this blot. Molecular weight is in kDa.

FIG. 31 shows a working model of how Tie-1 induces endothelial inflammation and may be crucial in atherosclerosis development.

FIGS. 32A-32B show thrombin activations of EphA2 in HUVECs. FIG. 32A is a western blot showing phosphorylated EphA2 levels. Phosphorylated EphA2 was detected by immunoprecipation of HUVEC cells treated with 1 U/ml thrombin for the indicated amount of time using an EphA2-polyclonal antibody in EphA2. Tyrosine phosphorylation was detected by western blot using the 4G10 anti-phosphotyrosine antibody. FIG. 32B bottom shows the same blot reprobed with EphA2-polyclonal antibody as a control for loading. Representative data from three independent experiments were shown.

FIGS. 33A-33B show thrombin induction of ICAM-1 upregulation in HUVECs is dependent on EphA2. FIG. 33A shows immunoblots of EphA2 (top), ICAM-1 (middle), and α-actinin for a protein loading control (bottom) in HUVECs treated with two human EphA2 specific siRNA as indicated and ICAM-1 upregulation was induced using 1 U/ml thrombin for 6 hours (T). Representative data from 3 experiments are shown. FIG. 33B is a graph showing densitometric quantification of the results in FIG. 34A normalized for fold increase in ICAM-1 expression. Results were reported as fold-increase in ICAM-1 expression relative to unstimulated controls. Data are mean±s.d. of three experiments; * p<0.002.

FIGS. 34A-34B show overexpression of mouse EphA2 rescues thrombin-induced ICAM-1 upregulation in HUVECs with endogenous EphA2 knocked down. FIG. 34A shows immunoblots of ICAM-1 (top), EphA2 (middle), and α-actinin for a protein loading control (bottom) using antibodies specific to EphA2, ICAM-1, and α-actinin, respectively in HUVECs stable expressing GFP (Control) or EphA2 via retroviral infection. The cells were treated with human EphA2 specific siRNA as indicated and ICAM-1 upregulation was induced using 1 U/ml thrombin for 6 hours (T). Representative data from 4 experiments are shown. FIG. 34B is an immunoblot showing soluble EphA2 failed to block thrombin-induced ICAM-1 upregulation. FIG. 35B top panel shows induction of ICAM-1 upregulation by thrombin is indifferent to soluble EphA2 concentration (FIG. 35B top panel lanes 0 to 1.0). “−” and “+” represent without or with thrombin stimulation (1 U/ml, 6 hr). Representative results from two experiments are shown.

FIGS. 35A-35B show endothelial EphA2 is required for mediating leukocyte attachment to thrombin-stimulated HUVECs. HUVECs stably overexpressing either GFP or mouse EphA2 and treated with either a control or a human EphA2 specific siRNA. The confluent HUVECs were stimulated with 5 U/ml thrombin for 6 hours. Fluorescently labeled U937 cells were then added. After one hour of incubation at room temperature on an orbital shaker, unattached cells were gently aspirated away. Cells were then fixed in 4% PFA. FIG. 35A is a series of fluorescent microscopy images of U937 cells as detected by fluorescence microscopy. Experiments were done in triplicate and representative results are shown. FIG. 35B is a graph quantifying the results in FIG. 35A. The number of attached U937 cells were counted in 4 randomly chosen fields of each experiments. Data are mean±standard deviation. Three experiments per condition were done. * p<0.005; #p<0.02.

FIG. 36 shows thrombin-induced tyrosine phosphorylation of EphA2 is dependent on Src kinase. Confluent HUVECs were pretreated with either DMSO or PP2 for 10 minutes and then stimulated with 1 U/ml thrombin or 250 ug/ml Ephrin A1-FC. EphA2 was immunoprecipitated and tyrosine phosphorylation was detected by immunoblot using an anti-phophotyrosine antibody (top panel). The blot was stripped and reblotted with the EphA2 antibody for loading (bottom panel). Representative data from 3 independent experiments are shown.

FIG. 37 is an immunoblot showing PAR-1 activates EphA2. Confluent HUVECs were stimulated with PAR agonistic peptides TFLLR-NH₂ (PAR-1) (SEQ ID NO: 11), RLLFT-NH₂ (negative control for PAR-1) (SEQ ID NO: 12), SLIGKV-NH₂ (PAR-2) (SEQ ID NO: 13), GYPGKF-NH₂ (PAR-4) (SEQ ID NO: 14), and thrombin. Tyrosine phosphorylation of EphA2 was determined by western blot.

FIG. 38 shows the results of an SH2 domain array experiment showing that thrombin-induced EphA2 activation has signaling consequences. Unstimulated and thrombin stimulated (1 U/ml, 5 mins) HUVEC lysates were analyzed by an SH2-domain array. Interactions of EphA2 to the SH2 domains of 38 signaling molecules were screened. Top, unstimulated; bottom, thrombin stimulated.

FIG. 39 is a schematic of a working model of how thrombin induces ICAM-1 expression in endothelial cells.

FIG. 40 shows the amino acid sequence of human Tie-1 (SEQ ID NO: 3).

FIG. 41 shows the nucleic acid sequence of human Tie-1 (SEQ ID NO: 4).

FIG. 42 shows the amino acid sequence of the human Tie-1 endodomain (SEQ ID NO: 5).

FIG. 43 shows the nucleic acid sequence of the human Tie-1 endodomain (SEQ ID NO: 6).

FIG. 44 shows the amino acid sequence of human thrombin (SEQ ID NO: 7).

FIG. 45 shows the nucleic acid sequence human thrombin (SEQ ID NO: 8).

FIG. 46 shows the amino acid sequence of human EphA2 (SEQ ID NO: 9).

FIG. 47 shows the nucleic acid sequence of human EphA2 (SEQ ID NO: 10).

Color versions of certain figures are present in U.S. patent application Ser. No. 12/592,034, which are hereby incorporated by reference.

### DETAILED DESCRIPTION

We have discovered signaling molecules, including Tie-1, Tie-1 endodomain, VEGFR2, VEGFR2 endodomain, EphA2, and fragments thereof, that are specifically involved in regulation of the pro-inflammatory effects of thrombin on endothelial cells and that inhibitors of such molecules can be used for the treatment of vascular inflammatory disorders or endothelial cell disorders and for the specific inhibition of the pro-inflammatory effects of thrombin.

In general, while not wishing to be bound by a particular theory, it is our hypothesis that, at arterial branch points, endothelial cells experience unusually high turbulent flow which upregulates Tie-1 expression and its activation, possibly through ectodomain shedding. Proinflammatory cytokines, such as IP-10, IL-6, and G-CSF, and adhesion molecules ICAM-1 and VCAM-1 are subsequently induced. These responses lead to recruitment and attachment of leukocytes from blood and proliferation and migration of smooth muscle cells in the intimal layer. Additionally, prothrombin to thrombin conversion is enhanced and locally generated thrombin may then activate PAR-1, which is abundantly expressed in endothelial cells. Activation of endothelial cells by thrombin not only induces upregulation of more inflammatory cytokines but also transactivates multiple receptor tyrosine kinases. Through the activity of VEGFR2, thrombin induces the dismantling of VE-cadherin complexes. Exposure of basal membrane components such as collagen or tissue factor due to endothelial gap formation further amplifies the inflammatory response. Since Tie-1 is one of the receptor tyrosine kinases that is transactivated by thrombin through PAR-1, an amplification loop may occur, ultimately leading to the development of a vascular inflammatory disorder such as atherosclerosis. These discoveries are described in detail below.

We have shown that the Tie-1 endodomain is biologically active and, using the active Tie-1 endodomain or overexpressing the full length Tie-1, we have discovered that Tie-1 is a critical upstream regulator of pathways that are associated with vascular inflammatory disorders or endothelial cell disorders such as atherosclerosis. We have discovered that Tie-1 stimulates expression of the cytokine markers IP-10, G-CSF, IL-6, VCAM-1, ICAM-1, CCL20, CCL2, CXCL5, E-selectin, p38 MAP kinase, and soluble CD44. Tie-1 also downregulates endothelial nitric oxide synthase (eNOS) expression. In addition, we have discovered that Tie-1 regulates the expression or biological activity of the genes indicated in the Appendix or the proteins encoded by these genes. We have also discovered that Tie-1 enhances attachment of monocytes to endothelial cells and smooth muscle cell migration. We have discovered that expression of activated Tie-1 promotes activation of thrombin and thrombin stimulation of endothelial cells through its receptor PAR-1 activates Tie-1. Activation of thrombin in an endothelial-cell specific manner in turn stimulates endothelial cells through PAR-1 and transactivates Tie-1. This scenario results in an amplification loop of endothelial inflammation which may trigger the onset of atherogenesis. We have also discovered that, in addition to the cytokine markers described above, activation of thrombin activates a number of signaling proteins in endothelial cells including receptor tyrosine kinases, VEGFR-2 endodomain, EGFR, insulin receptor, IGF-IR, AXL, HGFR (c-met), Flt-1, KDR, c-RET, MER, EphA2, and Tie-2.

Our discoveries provide a novel link between signaling molecules in endothelial cells. Endothelial cells are involved in both endothelial cell disorders and vascular inflammatory disorders; the latter also involves the action of additional cell types including smooth muscle cells. Therefore, the methods of the invention that include the downregulation of activated proteins identified herein and the upregulation of inhibited proteins described herein can be used to treat or prevent a vascular inflammatory disorder or an endothelial cell disorder.

According to the present invention, therapeutic compounds that inhibit the expression or biological activity of Tie-1, thrombin, tissue factor, any of the tyrosine kinase receptor proteins shown to be elevated or activated in the presence of thrombin (e.g., VEGFR-2, VEGFR-2 endodomain, EGFR, insulin receptor, IGF-IR, AXL, HGFR (c-met), Flt-1, KDR, c-RET, MER, EphA2, and Tie-2) or cytokines shown to be elevated or activated in the presence of activated Tie-1 or thrombin (e.g., ICAM-1, VCAM-1, IL-6, GCSF, tissue factor, CCL20, CCL2, CXCL5, soluble (alternatively spliced) CD44, and E-selectins), p38 MAP kinase, and any of the proteins shown to be upregulated in the Appendix, can be used to treat or prevent vascular inflammatory disorders or endothelial cell disorders. Therapeutic compounds that upregulate the expression or biological activity of proteins that were identified as inactive or downregulated in the presence of active Tie-1 (e.g., eNOS) can also be used for the treatment or prevention of vascular inflammatory disorders or endothelial cell disorders. Furthermore, Tie-1 inhibitor compounds and/or compounds that inhibit the upregulated tyrosine kinases in a cell or a subject in need thereof can be used to specifically inhibit the pro-atherogenic effects of thrombin without interfering with the ability of thrombin to promote fibrin conversion and clot formation. Examples of therapeutic inhibitor compounds and activator compounds are described in detail below.

It will be understood that the description of the inhibitor compounds provided below refer to compounds that can inhibit any of the polypeptides that are found to be upregulated in the presence of Tie-1 or thrombin, including Tie-1, Tie-endodomain, and thrombin. These polypeptides are collectively referred to as the activated polypeptides of the invention. The description of the activator compounds refer to compounds that can increase the expression or biological activity of any of the polypeptides that are found to be downregulated in the presence of Tie-1 or thrombin. These polypeptides are collectively referred to as the down-regulated polypeptides of the invention.

### Therapeutic Compounds

Therapeutic compounds useful in the methods of the invention include any compound that can reduce or inhibit the biological activity or expression level of any of the activated polypeptides of the invention and any compound that can increase the biological activity or expression level of any of the downregulated polypeptides of the invention.

Exemplary compounds that can increase the biological activity of expression level of the downregulated polypeptides of the invention include purified biologically active polypeptides of the invention (e.g., eNOS) and any peptidyl or non-peptidyl compound that specifically binds or activates the downregulated polypeptides of the invention (e.g., agonistic antibodies or antigen-binding fragments thereof).

Exemplary inhibitor compounds include, but are not limited to, purified biologically polypeptides of the invention that lack biological activity or biologically inactive fragments thereof, inhibitory fragments or mutants of the activated polypeptides of the invention (e.g., dominant negative fragments or fragments that lack biological activity, including the ability to bind substrate, kinase activity, and the ability to trigger signaling pathways); peptidyl or non-peptidyl compounds that specifically bind the activated polypeptides of the invention (e.g., antagonistic antibodies or antigen-binding fragments thereof); antisense nucleobase oligomers; morpholino oligonucleotides or any oligonucleotides which target the translation start sequence or splicing sequence of the mRNA of the invention; small RNAs; small molecule inhibitors; compounds that decrease the half-life of the mRNA or protein of any of the activated polypeptides of the invention; compounds that decrease transcription or translation of any of the activated polypeptides of the invention; compounds that reduce or inhibit the expression levels of any of the activated polypeptides of the invention or decrease the biological activity of any of the activated polypeptides of the invention; compounds that alter expression or biological activity of proteins downstream of for example, Tie-1, thrombin, EphA2, or any of the activated polypeptides of the invention. Examples of small RNAs and antibodies are provided in the Examples below.

As described above, the inhibitor compounds can be used to reduce or inhibit the expression or biological activity of any one or more of the activated polypeptides of the invention including, but not limited to, Tie-1, tissue factor, thrombin, IP-10, G-CSF, IL-6, VCAM-1, ICAM-1, CCL20, CCL2, CXCL5, E-selectin, p38 MAP kinase, soluble CD44, VEGFR-2 endodomain, EGFR, insulin receptor, IGF-IR, AXL, HGFR (c-met), Flt-1, KDR, c-RET, MER, EphA2, Tie-2 and any of the proteins shown to be upregulated in the Appendix. In one example, a Tie-1 inhibitor compound is used to inhibit the biological activity of thrombin or any of the proteins that are regulated by expression of activated Tie-1.

Desirably, the inhibitor compounds will reduce or inhibit the expression or biological activity of Tie-1, Tie-1 endodomain, thrombin, VEGFR2, VEGFR2 endodomain, or EphA2. Inhibitor compounds that inhibit Tie-1 may, for example, inhibit Tie-1 kinase activity, inhibit phosphorylation of the Tie-1 endodomain, inhibit Tie-1-mediated endothelial cell adhesion, inhibit Tie-1-mediated smooth muscle cell migration, inhibit cleavage of Tie-1 or shedding of the Tie-1 ectodomain, or inhibit activation of one or more cytokine or inflammatory markers. One example of a Tie-1 inhibitor compound is a peptidyl or non-peptidyl compound (e.g., antibodies or antigen binding fragments thereof) that specifically bind Tie-1, for example, the Tie-1 endodomain or the ATP binding pocket of Tie-1. Another example of a Tie-1 inhibitor compound is a dominant negative Tie-1 protein that does not induce Tie-1 biological activity. Another example of a Tie-1 inhibitor compound is an antagonistic ligand that binds to but does not activate Tie-1 signaling. Additional examples include antisense nucleobase oligomers, morpholinos, or small RNAs that are substantially identical to at least a portion of a Tie-1 nucleic acid sequence or complementary sequence thereof (SEQ ID NOs: 4 and 6). Tie-1 inhibitor compounds may also inhibit any of the characteristics of vascular inflammation including endothelial cell dysfunction, smooth muscle cell proliferation or migration, and endothelial cell attachment.

Tie-1 inhibitor compounds may not only inhibit Tie-1 expression or biological activity but may also inhibit thrombin biological activity. Desirably, the Tie-1 inhibitor compound specifically inhibits the pro-inflammatory or pro-atherosclerotic activity of thrombin but not the pro-coagulant activity of thrombin.

Tie-1 inhibitor compounds that inhibit thrombin may, for example, reduce or inhibit thrombin induced endothelial cell permeability; thrombin mediated phosphorylation or activation of signaling proteins including, but not limited to, VEGFR2 or VEGFR2 endodomain, MLC, VE cadherin, and p120; and thrombin induced intracellular gap formation. Desirably, a thrombin inhibitor compound will specifically inhibit the proinflammatory activity and not the ability of thrombin to promote fibrin clot formation.

Exemplary inhibitor compounds that inhibit VEGFR2 or VEGFR2 endodomain may, for example, inhibit VEGFR2 or VEGFR2 endodomain mediated kinase activity, inhibit substrate binding, wherein the substrate may or may not be VEGF, inhibit endothelial cell permeability, or inhibit intracellular gap formation. Additional exemplary inhibitor compounds that inhibit VEGFR2 or VEGFR2 endodomain may, for example, inhibit the biological activities of VEGFR2 known in the art including promoting angiogenesis and proliferation.

Inhibitor compounds that inhibit EphA2 may, for example, reduce or inhibit EphA2 pro-inflammatory activity; ligand binding (non-limiting examples of ligands include thrombin, as described herein, and Ephrin A1); kinase activity including but not limited to Ephrin A1 dependent and independent kinase activity, EphA2 mediated Src dependent and independent kinase activity, wherein the phosphorylation can be autophosphorylation or phosphorylation of another substrate such as other Eph proteins; interaction with other proteins such as Src, FAK, and SH2 domain containing proteins (e.g., CkrL, PI3K (both α and β subunits) and SHP-2); changes in localization; activation or elevation of signaling pathways such the Ras-MAPK and Rho GTP-ase signaling pathways; and modulation of ICAM-1 activation. Non-limiting examples of EphA2 inhibits include dasatinib and green tea catechin (Tang et al., J. Nutr. Biochem 18:391-399 (2007)).

Desirably, inhibitor compounds will reduce or inhibit the biological activity or expression levels of an activated polypeptide of the invention by at least 10%, 25%, 30%, 40%, 50%, 60%, 70%, 80%, 90%, 95%, 96%, 97%, 98%, 99%, or more. Preferably, the inhibitor compound can reduce or inhibit angiogenesis, smooth muscle cell proliferation, endothelial cell dysfunction, inflammation, endothelial cell permeability, or inhibit intracellular gap formation, calcification, neointimal hyperplasia, arteriosclerosis, or atherosclerosis by at least 10%, 20%, 25%, 30%, 40%, 50%, 60%, 70%, 80%, 90%, 95%, 96%, 97%, 98%, 99%, or more.

### Polypeptides

Therapeutic compounds of the invention that include polypeptides can be used as inhibitor or activator compounds in the methods of the invention. Preferred polypeptides that can be used as inhibitor compounds include dominant negative fragments or mutants of the activated polypeptides of the invention that bind to functional regions of the polypeptide (e.g., the ATP binding pocket for kinases or the substrate binding domains). By binding to the functional region, the polypeptide can inhibit the activity of the targeted polypeptide presumably by steric interference. In one example, a kinase deficient form of a kinase can act as a dominant negative polypeptide. Purified polypeptides can also be used an agonist for the upregulation of a downregulated polypeptide of the invention (e.g., eNOS).

Any polypeptide (including antibodies or fragments thereof) that is used in the methods of the invention can be produced, purified, and/or modified using any of the methods and modifications known in the art or described herein. Examples of polypeptide modifications include phosphorylation, acylation, glycosylation, pegylation (e.g., addition of polyethylene glycol), sulfation, prenylation, methylation, hydroxylation, carboxylation, and amidation. Additional examples of polypeptide modifications are provided in WO 2007/033216, herein incorporated by reference.

The ability of any of the above polypeptides to function as an inhibitor or activator compound may be tested according to any of the assays described in the Examples.

### Antibodies

Antibodies that specifically bind to any of the polypeptides of the invention, have a high affinity (K_{D}<500 nM) for the polypeptide (e.g., Tie-1, Tie-1 endodomain, VEGFR2 endodomain, EphA2, and any of the cytokines or kinases shown to be upregulated by Tie-1 or thrombin) and desirably neutralize or prevent the biological activity of the polypeptide are useful in the therapeutic methods of the invention. In one embodiment, the antibody, or fragment or derivative thereof, binds to the ATP binding pocket of a kinase (e.g., VEGFR2, VEGFR2 endodomain, or EphA2) or substrate binding domain. Non-limiting examples of antibodies that specifically block one or more of the activated polypeptides of the invention are provided in the Examples below. The antibodies useful in the methods of the present invention include, without limitation, anti-monoclonal, polyclonal, chimeric, and humanized antibodies and functional equivalents or derivatives of antibodies as described below.

Pharmaceutical compositions, for example, including excipients, of any antibodies of the invention are also included. Methods for the preparation and use of antibodies for therapeutic purposes are described in several patents including U.S. Pat. Nos. 6,054,297; 5,821,337; 6,365,157; and 6,165,464.

### Monoclonal and Polyclonal Antibodies

Monoclonal and polyclonal antibodies useful in the methods of the invention may be produced by methods known in the art. These methods include the immunological method described by Kohler and Milstein (Nature, 256: 495-497, 1975), Kohler and Milstein (Eur. J. Immunol, 6, 511-519, 1976), and Campbell (“Monoclonal Antibody Technology, The Production and Characterization of Rodent and Human Hybridomas” in Burdon et al., Eds., Laboratory Techniques in Biochemistry and Molecular Biology, Volume 13, Elsevier Science Publishers, Amsterdam, 1985), as well as by the recombinant DNA method described by Huse et al. (Science, 246, 1275-1281, 1989).

Human or humanized antibodies can also be produced using phage display libraries (Marks et al., J. Mol. Biol., 222:581-597, 1991 and Winter et al. Annu. Rev. Immunol., 12:433-455, 1994). The techniques of Cole et al. and Boerner et al. are also useful for the preparation of human or humanized monoclonal antibodies (Cole et al., supra; Boerner et al., J. Immunol., 147: 86-95, 1991).

Monoclonal antibodies are isolated and purified using standard art-known methods. For example, antibodies can be screened using standard art-known methods such as ELISA or Western blot analysis. Non-limiting examples of such techniques are described in Examples II and III of U.S. Pat. No. 6,365,157, herein incorporated by reference.

### Chimeric Antibodies

The art has attempted to overcome the problem of rodent antibody-induced anti-globulin response by constructing “chimeric” antibodies in which an animal antigen-binding variable domain is coupled to a human constant domain (U.S. Pat. No. 4,816,567; Morrison et al., Proc. Natl. Acad. Sci. USA, 81:6851-6855, 1984; Boulianne et al., Nature, 312:643-646, 1984; Neuberger et al., Nature, 314:268-270, 1985; and PCT publication no. WO 2005/012359). Chimerized antibodies preferably have constant regions derived substantially or exclusively from human antibody constant regions and variable regions derived substantially or exclusively from the sequence of the variable region from a mammal other than a human.

### Humanized Antibodies

Humanized antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Methods for humanizing non-human antibodies are well known in the art (for reviews see Vaswani and Hamilton, Ann. Allergy Asthma Immunol., 81:105-119, 1998 and Carter, Nature Reviews Cancer, 1:118-129, 2001). Generally, a humanized antibody has one or more amino acid residues introduced into it from a source that is non-human. These non-human amino acid residues are often referred to as import residues, which are typically taken from an import variable domain.

Humanization of an antibody can be essentially performed following the methods known in the art (Jones et al., Nature, 321:522-525, 1986; Riechmann et al., Nature, 332:323-329, 1988; Verhoeyen et al., Science, 239:1534-1536 1988; and PCT publication no. WO 2005/012359), by substituting rodent CDRs or other CDR sequences for the corresponding sequences of a human antibody. Accordingly, such humanized antibodies are chimeric antibodies wherein substantially less than an intact human variable domain has been substituted by the corresponding sequence from a non-human species (see for example, U.S. Pat. No. 4,816,567). In practice, humanized antibodies are typically human antibodies in which some CDR residues and possibly some framework residues are substituted by residues from analogous sites in rodent antibodies (Presta, Curr. Op. Struct. Biol., 2:593-596, 1992). Additional methods for the preparation of humanized antibodies can be found in U.S. Pat. Nos. 5,821,337, and 6,054,297, and Carter, (supra) which are all incorporated herein by reference. The humanized antibody is selected from any class of immunoglobulins, including IgM, IgG, IgD, IgA and IgE, and any isotype, including IgG₁, IgG₂, IgG₃, and IgG₄. Where cytotoxic activity is not needed, such as in the present invention, the constant domain is preferably of the IgG₂ class. The humanized antibody may comprise sequences from more than one class or isotype, and selecting particular constant domains to optimize desired effector functions is within the ordinary skill in the art.

### Functional Equivalents or Derivatives of Antibodies

The invention also includes functional equivalents or derivatives of the antibodies described in this specification. Functional equivalents or derivatives include polypeptides with amino acid sequences substantially identical to the amino acid sequence of the variable or hypervariable regions of the antibodies of the invention. Functional equivalents have binding characteristics comparable to those of the antibodies, and include, for example, chimerized, humanized and single chain antibodies or fragments thereof, diabodies, linear antibodies, antibody fragments (e.g., Fab fragments, F(ab′)₂ fragments, Fv fragments), and antibodies, or fragments thereof, fused to a second protein, or fragment thereof. Methods of producing such functional equivalents are disclosed, for example, in PCT Publication No. WO93/21319; European Patent Application No. 239,400; PCT Publication No. WO89/09622; European Patent Application No. 338,745; European Patent Application No. 332424; a U.S. Pat. No. 4,816,567; and PCT publication no. WO 2005/012359, each of which is herein incorporated by reference.

Functional equivalents of antibodies also include single-chain antibody fragments, also known as single-chain antibodies (scFvs). Single-chain antibody fragments are recombinant polypeptides which typically bind antigens or receptors; these fragments contain at least one fragment of an antibody variable heavy-chain amino acid sequence (V_{H}) tethered to at least one fragment of an antibody variable light-chain sequence (V_{L}) with or without one or more interconnecting linkers. Such a linker may be a short, flexible peptide selected to assure that the proper three-dimensional folding of the V_{L} and V_{H} domains occurs once they are linked so as to maintain the target molecule binding-specificity of the whole antibody from which the single-chain antibody fragment is derived. Generally, the carboxyl terminus of the V_{L} or V_{H} sequence is covalently linked by such a peptide linker to the amino acid terminus of a complementary V_{L} and V_{H} sequence. Single-chain antibody fragments can be generated by molecular cloning, antibody phage display library or similar techniques. These proteins can be produced either in eukaryotic cells or prokaryotic cells, including bacteria.

Single-chain antibody fragments contain amino acid sequences having at least one of the variable regions or CDRs of the whole antibodies described in this specification, but are lacking some or all of the constant domains of those antibodies. These constant domains are not necessary for antigen binding, but constitute a major portion of the structure of whole antibodies. Single-chain antibody fragments may therefore overcome some of the problems associated with the use of antibodies containing part or all of a constant domain. For example, single-chain antibody fragments tend to be free of undesired interactions between biological molecules and the heavy-chain constant region, or other unwanted biological activity. Additionally, single-chain antibody fragments are considerably smaller than whole antibodies and may therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely than whole antibodies to provoke an immune response in a recipient.

Further, the functional equivalents may be or may combine members of any one of the following immunoglobulin classes: IgG, IgM, IgA, IgD, or IgE, and the subclasses thereof.

Equivalents of antibodies are prepared by methods known in the art. For example, fragments of antibodies may be prepared enzymatically from whole antibodies. Preferably, equivalents of antibodies are prepared from DNA encoding such equivalents. DNA encoding fragments of antibodies may be prepared by deleting all but the desired portion of the DNA that encodes the full-length antibody.

### Nucleic Acid Molecules

The present invention features nucleic acid molecules encoding a down-regulated polypeptide of the invention which can be used for the treatment or prevention of a vascular inflammatory disorder. The present invention also features inhibitory nucleic acid molecules which can be used for the treatment or prevention of a vascular inflammatory disorder. Such inhibitory nucleic acid molecules are capable of mediating downregulation of the expression of an activated polypeptide of the invention or nucleic acid encoding the same or mediating a decrease in the activity of an activated polypeptide of the invention. Examples of the inhibitory nucleic acids of the invention include, without limitation, antisense oligomers (e.g., morpholinos), dsRNAs (e.g., siRNAs and shRNAs), and aptamers. Each of these is described in detail below.

### Antisense Oligomers

The present invention features antisense nucleobase oligomers to any of the activated polypeptides of the invention (e.g., Tie-1, Tie-2, tissue factor, thrombin, IP-10, G-CSF, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, KDR, VEGFR2 endodomain, c-RET, MER, and EphA2) and the use of such oligomers to downregulate expression of mRNA encoding the polypeptide. By binding to the complementary nucleic acid sequence (the sense or coding strand), antisense nucleobase oligomers are able to inhibit protein expression presumably through the enzymatic cleavage of the RNA strand by RNAse H. Desirably, the antisense nucleobase oligomer is capable of reducing activated polypeptide expression in a cell that expresses increased levels of the activated polypeptide of the invention by at least 10% relative to cells treated with a control oligonucleotide, preferably 20% or greater, more preferably 40%, 50%, 60%, 70%, 80%, 90% or greater. Methods for selecting and preparing antisense nucleobase oligomers are well known in the art. Methods for assaying levels of protein expression are also well known in the art and include Western blotting, immunoprecipitation, and ELISA.

One example of an antisense nucleobase oligomer particularly useful in the methods and compositions of the invention is a morpholino oligomer. Morpholinos are used to block access of other molecules to specific sequences within nucleic acid molecules. They can block access of other molecules to small (˜25 base) regions of ribonucleic acid (RNA). Morpholinos are sometimes referred to as PMO, an acronym for phosphorodiamidate morpholino oligo.

Morpholinos are used to knock down gene function by preventing cells from making a targeted protein or by modifying the splicing of pre-mRNA. Morpholinos are synthetic molecules that bind to complementary sequences of RNA by standard nucleic acid base-pairing. While morpholinos have standard nucleic acid bases, those bases are bound to morpholine rings instead of deoxyribose rings and linked through phosphorodiamidate groups instead of phosphates. Replacement of anionic phosphates with the uncharged phosphorodiamidate groups eliminates ionization in the usual physiological pH range, so morpholinos in organisms or cells are uncharged molecules.

Morpholinos act by “steric blocking” or binding to a target sequence within an RNA and blocking molecules which might otherwise interact with the RNA. Because of their completely unnatural backbones, morpholinos are not recognized by cellular proteins. Nucleases do not degrade morpholinos and morpholinos do not activate toll-like receptors and so they do not activate innate immune responses such as the interferon system or the NF-κB-mediated inflammation response. Morpholinos are also not known to modify methylation of DNA. Therefore, morpholinos directed to any part of an activated polypeptide of the invention (e.g., Tie-1, Tie-2, tissue factor, thrombin, IP-10, G-CSF, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, KDR, VEGFR2 endodomain, c-RET, MER, and EphA2) and that reduce or inhibit the expression levels or biological activity of the activated polypeptide of the invention are particularly useful in the methods and compositions of the invention that require the use of inhibitor compounds. For example, morpholinos may be targeted to both the coding and non-coding sequences of an mRNA (e.g., Tie-1, Tie-2, tissue factor, thrombin, IP-10, G-CSF, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, KDR, VEGFR2 endodomain, c-RET, MER, and EphA2). In desired embodiments, the morpholino is targeted to Tie-1, Tie-1 endodomain, thrombin, VEGFR2 or VEGFR2 endodomain, or EphA2 mRNA. In preferred embodiments, the morpholinos may be designed to target the ATG or translation start site or a intron/exon splice site within the sequence of an mRNA (e.g., Tie-1, Tie-2, tissue factor, thrombin, IP-10, G-CSF, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, KDR, VEGFR2 endodomain, c-RET, MER, and EphA2).

dsRNAs

The present invention also features the use of double stranded RNAs including, but not limited to siRNAs and shRNAs. Short double-stranded RNAs may be used to perform RNA interference (RNAi) to inhibit expression of an activated polypeptide of the invention. RNAi is a form of post-transcriptional gene silencing initiated by the introduction of double-stranded RNA (dsRNA). Short 15 to 32 nucleotide double-stranded RNAs, known generally as “siRNAs,” “small RNAs,” or “microRNAs” are effective at down-regulating gene expression in nematodes (Zamore et al., Cell 101: 25-33) and in mammalian tissue culture cell lines (Elbashir et al., Nature 411:494-498, 2001). The further therapeutic effectiveness of this approach in mammals was demonstrated in vivo by McCaffrey et al. (Nature 418:38-39. 2002). The small RNAs are at least 15 nucleotides, preferably, 17, 18, 19, 20, 21, 22, 23, 24, 25, 26, 27, 28, 29, 30, 31, 32, 33, 34, 35, nucleotides in length and even up to 50 or 100 nucleotides in length (inclusive of all integers in between). Such small RNAs that are substantially identical to or complementary to any region of an activated polypeptide of the invention are included in the invention. Examples are provided in the Examples section, below. Non-limiting examples of desirable small RNAs are substantially identical (e.g., 60%, 65%, 70%, 75%, 80%, 85%, 90%, 95%, 96%, 97%, 98%, 99%, or 100% sequence identity) to or complementary to the Tie-1, Tie-1 endodomain, thrombin, VEGFR2, VEGFR2 endodomain, or EphA2 sequence (see SEQ ID NOs: 1-10) including the translational start sequence or the splicing sequence. Non-limiting examples of siRNA molecules that can be used in the methods of the invention are described in the Examples below.

The invention includes any small RNA substantially identical to at least 15 nucleotides, preferably, 17, 18, 19, 20, 21, 22, 23, 24, 25, 26, 27, 28, 29, 30, 31, 32, 33, 34, or 35, nucleotides in length and even up to 50 or 100 nucleotides in length (inclusive of all integers in between) of any region of SEQ ID NOs: 1-10. It should be noted that longer dsRNA fragments can be used that are processed into such small RNAs. Useful small RNAs can be identified by their ability to decrease polypeptide expression levels or biological activity using, for example, assays known in the art or provided herein. Small RNAs can also include short hairpin RNAs in which both strands of an siRNA duplex are included within a single RNA molecule.

The specific requirements and modifications of small RNA are known in the art and are described, for example, in PCT Publication No. WO01/75164, and U.S. Application Publication Nos. 20060134787, 20050153918, 20050058982, 20050037988, and 20040203145, the relevant portions of which are herein incorporated by reference. In particular embodiments, siRNAs can be synthesized or generated by processing longer double-stranded RNAs, for example, in the presence of the enzyme dicer under conditions in which the dsRNA is processed to RNA molecules of about 17 to about 26 nucleotides. siRNAs can also be generated by expression of the corresponding DNA fragment (e.g., a hairpin DNA construct). Generally, the siRNA has a characteristic 2- to 3-nucleotide 3′ overhanging ends, preferably these are (2′-deoxy)thymidine or uracil. The siRNAs typically comprise a 3′ hydroxyl group. In some embodiments, single stranded siRNAs or blunt ended dsRNA are used. In order to further enhance the stability of the RNA, the 3′ overhangs are stabilized against degradation. In one embodiment, the RNA is stabilized by including purine nucleotides, such as adenosine or guanosine. Alternatively, substitution of pyrimidine nucleotides by modified analogs, e.g., substitution of uridine 2-nucleotide overhangs by (2′-deoxy)thymide is tolerated and does not affect the efficiency of RNAi. The absence of a 2′ hydroxyl group significantly enhances the nuclease resistance of the overhang in tissue culture medium.

siRNA molecules can be obtained through a variety of protocols including chemical synthesis or recombinant production using a Drosophila in vitro system. They can be commercially obtained from companies such as Dharmacon Research Inc. or Xeragon Inc., or they can be synthesized using commercially available kits such as the Silencer™ siRNA Construction Kit from Ambion (catalog number 1620) or HiScribe™ RNAi Transcription Kit from New England BioLabs (catalog number E2000S).

Alternatively siRNA can be prepared using standard procedures for in vitro transcription of RNA and dsRNA annealing procedures such as those described in Elbashir et al. (Genes & Dev., 15:188-200, 2001), Girard et al. (Nature 442:199-202, 2006), Aravin et al. (Nature 442:203-207, 2006), Grivna et al. (Genes Dev. 20:1709-1714, 2006), and Lau et al. (Science 313:305-306, 2006). siRNAs are also obtained by incubation of dsRNA that corresponds to a sequence of the target gene in a cell-free Drosophila lysate from syncytial blastoderm Drosophila embryos under conditions in which the dsRNA is processed to generate siRNAs of about 21 to about 23 nucleotides, which are then isolated using techniques known to those of skill in the art. For example, gel electrophoresis can be used to separate the 21-23 nt RNAs and the RNAs can then be eluted from the gel slices. In addition, chromatography (e.g., size exclusion chromatography), glycerol gradient centrifugation, and affinity purification with antibody can be used to isolate the small RNAs.

Short hairpin RNAs (shRNAs), as described in Yu et al. (Proc. Natl. Acad. Sci. USA, 99:6047-6052, 2002) or Paddison et al. (Genes & Dev, 16:948-958, 2002), incorporated herein by reference, can also be used in the methods of the invention. shRNAs are designed such that both the sense and antisense strands are included within a single RNA molecule and connected by a loop of nucleotides (3 or more). shRNAs can be synthesized and purified using standard in vitro T7 transcription synthesis as described above and in Yu et al. (supra). shRNAs can also be subcloned into an expression vector that has the mouse U6 promoter sequences which can then be transfected into cells and used for in vivo expression of the shRNA.

A variety of methods are available for transfection, or introduction, of dsRNA into mammalian cells. For example, there are several commercially available transfection reagents useful for lipid-based transfection of siRNAs including but not limited to: TransIT-TKO™ (Minis, Cat. #MIR 2150), Transmessenger™ (Qiagen, Cat. #301525), Oligofectamine™ and Lipofectamine™ (Invitrogen, Cat. #MIR 12252-011 and Cat. #13778-075), siPORT™ (Ambion, Cat. #1631), DharmaFECT™ (Fisher Scientific, Cat. #T-2001-01). Agents are also commercially available for electroporation-based methods for transfection of siRNA, such as siPORTer™ (Ambion Inc. Cat. #1629). Microinjection techniques can also be used. The small RNA can also be transcribed from an expression construct introduced into the cells, where the expression construct includes a coding sequence for transcribing the small RNA operably linked to one or more transcriptional regulatory sequences. Where desired, plasmids, vectors, or viral vectors can also be used for the delivery of dsRNA or siRNA and such vectors are known in the art. Protocols for each transfection reagent are available from the manufacturer. Additional methods are known in the art and are described, for example in U.S. Patent Application Publication No. 20060058255.

### Aptamers

The present invention also features aptamers to the activated polypeptides of the invention and the use of such aptamers to downregulate expression of the activated polypeptide or nucleic acid encoding the polypeptide. Aptamers are nucleic acid molecules that form tertiary structures that specifically bind to a target molecule. The generation and therapeutic use of aptamers are well established in the art. See, e.g., U.S. Pat. No. 5,475,096. For example, a Tie-1 aptamer may be a pegylated modified oligonucleotide, which adopts a three-dimensional conformation that enables it to bind to Tie and inhibit the biological activity of Tie-1. Additional information on aptamers can be found, for e.g., in U.S. Patent Application Publication No. 20060148748.

### Disorders

We have discovered signaling molecules, including Tie-1, Tie-endodomain, thrombin, tissue factor, VEGFR2, VEGFR2 endodomain, EphA2, and fragments thereof, that are specifically involved in regulation of the pro-inflammatory effects of thrombin on endothelial cells and that inhibitors of such molecules can be used for the treatment of vascular inflammatory disorders or endothelial cell disorders. In addition, we have discovered that eNOS expression is downregulated and activators of eNOS can be used in combination with any of the inhibitor compounds of the invention for the treatment of vascular inflammatory disorders or endothelial cell disorders.

The vascular inflammatory disorders that can be treated by the methods of the invention include any disorder of the vasculature that includes one or more of the following characteristics: endothelial cell dysfunction, increased angiogenesis, calcification, increased smooth muscle cell proliferation, increased attachment of leukocytes, and increased infiltration of leukocytes such as monocytes, T cells, and foamy macrophages. Preferably, the vascular inflammatory disorder includes at least two, at least three, or at least four or more of the above characteristics. Endothelial cell dysfunction is determined using assays known in the art including detecting the increased expression of endothelial adhesion molecules or decreased expression or biological activity of nitric oxide synthase. Angiogenesis is measured using a variety of angiogenesis assays known in the art including the detection of pro-angiogenic markers, such as VEGF or VEGF receptors, and the chicken chorioallantoic membrane assay. Smooth muscle cell proliferation is measured by the increased presence of smooth muscle cells or SM-like cells identified by markers such as smooth muscle cell actin and desmin. Desirable therapeutic inhibitor or activator compounds used for the treatment of a vascular inflammatory disorder in the methods of the invention will reduce or inhibit any one or more of the characteristics of a vascular inflammatory disorder or will reduce or inhibit any one or more of the symptoms of a vascular inflammatory disorder.

Examples of vascular inflammatory disorders include arteriosclerosis (acute or chronic), atherosclerosis (acute or chronic), and neointimal hyperplasia (e.g., venous neointimal hyperplasia, peripheral vascular disease, and dialysis vascular access).

The endothelial cell disorders that can be treated by the methods of the invention include any disorder that is characterized by endothelial cell dysfunction. Non-limiting examples of diseases or disorders that are characterized by endothelial cell dysfunction include angiogenic disorders such as cancers which require neovascularization to support tumor growth, infectious diseases, autoimmune disorders, vascular malformations, DiGeorge syndrome, HHT, cavernous hemangioma, transplant arteriopathy, vascular access stenosis associated with hemodialysis, vasculitis, vasculitidis, vascular inflammatory disorders, atherosclerosis, obesity, psoriasis, warts, allergic dermatitis, scar keloids, pyogenic granulomas, blistering disease, Kaposi sarcoma, persistent hyperplastic vitreous syndrome, retinopathy of prematurity, choroidal neovascularization, macular degeneration, diabetic retinopathy, ocular neovascularization, primary pulmonary hypertension, asthma, nasal polyps, inflammatory bowel and periodontal disease, ascites, peritoneal adhesions, contraception, endometriosis, uterine bleeding, ovarian cysts, ovarian hyperstimulation, arthritis, rheumatoid arthritis, chronic articular rheumatism, synovitis, osteoarthritis, osteomyelitis, osteophyte formation, sepsis, and vascular leak. Endothelial cell dysfunction can be determined using assays known in the art including detecting the increased expression of endothelial adhesion molecules or decreased expression or biological activity of nitric oxide synthase (eNOS).

### Therapeutic Formulations

The invention includes the use of therapeutic compounds (e.g., inhibitor compounds or activator compounds) to treat, prevent, or reduce the risk of developing a vascular inflammatory disorder or an endothelial cell disorder in a subject. The therapeutic compound can be administered at anytime. For example, for therapeutic applications the compound can be administered after diagnosis or detection of a vascular inflammatory disorder or an endothelial cell disorder or after the onset of symptoms of a vascular inflammatory disorder or an endothelial cell disorder. The therapeutic compound can also be administered before diagnosis or onset of symptoms for prevention of a vascular inflammatory disorder or an endothelial cell disorder in subjects that have not yet been diagnosed with a vascular inflammatory disorder or an endothelial cell disorder but are at risk of developing such a disorder, or after a risk of developing a vascular inflammatory disorder or an endothelial cell disorder is determined. A therapeutic compound of the invention (e.g., inhibitor compound or activator compound) may be formulated with a pharmaceutically-acceptable diluent, carrier, or excipient, in unit dosage form. Conventional pharmaceutical practice may be employed to provide suitable formulations or compositions to administer the therapeutic compound of the invention to a patient suffering from or at risk of developing a vascular inflammatory disorder or an endothelial cell disorder. Administration may begin before the patient is symptomatic. The therapeutic compound of the present invention can be formulated and administered in a variety of ways, e.g., those routes known for specific indications, including, but not limited to, topically, orally, subcutaneously, intravenously, intracerebrally, intranasally, transdermally, intraperitoneally, intramuscularly, intrapulmonary, rectally, intraarterially, intralesionally, parenterally, or intraocularly. The therapeutic compound can be in the form of a pill, tablet, capsule, liquid, or sustained release tablet for oral administration; or a liquid for intravenous administration, subcutaneous administration, or injection; for intranasal formulations, in the form of powders, nasal drops, or aerosols; or a polymer or other sustained-release vehicle for local administration.

The invention also includes the use of therapeutic compound to treat, prevent, or reduce the risk of developing a vascular inflammatory disorder or an endothelial cell disorder in a biological sample derived from a subject (e.g., treatment of a biological sample ex vivo) using any means of administration and formulation described herein. The biological sample to be treated ex vivo may include any biological fluid (e.g., blood, serum, plasma, or cerebrospinal fluid), cell (e.g., an endothelial cell), or tissue (e.g., vascular tissue) from a subject that has a vascular inflammatory disorder or an endothelial cell disorder or the propensity to develop a vascular inflammatory disorder or an endothelial cell disorder. The biological sample treated ex vivo with the therapeutic compound may be reintroduced back into the original subject or into a different subject. The ex vivo treatment of a biological sample with a therapeutic compound, as described herein, may be repeated in an individual subject (e.g., at least once, twice, three times, four times, or at least ten times). Additionally, ex vivo treatment of a biological sample derived from a subject with a therapeutic compound, as described herein, may be repeated at regular intervals (non-limiting examples include daily, weekly, monthly, twice a month, three times a month, four times a month, bi-monthly, once a year, twice a year, three times a year, four times a year, five times a year, six times a year, seven times a year, eight times a year, nine times a year, ten times a year, eleven times a year, and twelve times a year).

Therapeutic formulations are prepared using standard methods known in the art by mixing the active ingredient having the desired degree of purity with optional physiologically acceptable carriers, excipients or stabilizers (Remington's Pharmaceutical Sciences (20th edition), ed. A. Gennaro, 2000, Lippincott, Williams & Wilkins, Philadelphia, Pa.), in the form of lyophilized formulations or aqueous solutions. Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, but preferably, the formulation contains a pharmaceutically acceptable salt, preferably sodium chloride, and preferably at about physiological concentrations. The formulation may also contain the inhibitor compound in the form of a calcium salt. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant. Preferred surfactants are non-ionic detergents.

For parenteral administration, the therapeutic compound is formulated in a unit dosage injectable form (solution, suspension, emulsion) in association with a pharmaceutically acceptable parenteral vehicle. Such vehicles are inherently nontoxic and non-therapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives.

The dosage required depends on the choice of the route of administration; the nature of the formulation; the nature of the subject's illness; the subject's size, weight, surface area, age, and sex; other drugs being administered; and the judgment of the attending physician. For example, oral administration would be expected to require higher dosages than administration by intravenous injection. Variations in these dosage levels can be adjusted using standard empirical routines for optimization as is well understood in the art. Administrations can be single or multiple (e.g., 2-, 3-, 6-, 8-, 10-, 20-, 50-, 100-, 150-, or more). Encapsulation of the therapeutic compound in a suitable delivery vehicle (e.g., polymeric microparticles or implantable devices) may increase the efficiency of delivery, particularly for oral delivery.

As described above, the dosage of the therapeutic compound will depend on other clinical factors such as weight and condition of the subject and the route of administration of the compound. For treating subjects, between approximately 0.001 mg/kg to 500 mg/kg body weight of the inhibitor compound can be administered. A more preferable range is 0.01 mg/kg to 50 mg/kg body weight with the most preferable range being from 1 mg/kg to 25 mg/kg body weight. Depending upon the half-life of the therapeutic compound in the particular subject, the compound can be administered between several times per day to once a week. The methods of the present invention provide for single as well as multiple administrations, given either simultaneously or over an extended period of time.

Alternatively, a polynucleotide containing a nucleic acid sequence which is itself or encodes a therapeutic compound (e.g., an inhibitory nucleic acid molecule that inhibits the expression of a nucleic acid molecule encoding an activated polypeptide of the invention or the biological activity of the activated polypeptide of the invention or a nucleic acid molecule that encodes a downregulated polypeptide of the invention) can be delivered to the appropriate cells in the subject. Expression of the coding sequence can be directed to any cell in the body of the subject, preferably an endothelial cell. This can be achieved, for example, through the use of polymeric, biodegradable microparticle or microcapsule delivery devices known in the art.

The nucleic acid can be introduced into the cells by any means appropriate for the vector employed. Many such methods are well known in the art (Sambrook et al., supra, and Watson et al., Recombinant DNA, Chapter 12, 2d edition, Scientific American Books, 1992). Examples of methods of gene delivery include liposome-mediated transfection, electroporation, calcium phosphate/DEAE dextran methods, gene gun, and microinjection. Delivery of “naked DNA” (i.e., without a delivery vehicle) to an intramuscular, intradermal, or subcutaneous site is another means to achieve in vivo expression. Gene delivery using viral vectors such as adenoviral, retroviral, lentiviral, or adeno-associated viral vectors can also be used. An ex vivo strategy can also be used for therapeutic applications. Ex vivo strategies involve transfecting or transducing cells obtained from the subject with a therapeutic nucleic acid compound. The transfected or transduced cells are then returned to the subject. Such cells act as a source of the therapeutic nucleic acid compound for as long as they survive in the subject.

Therapeutic compounds (e.g., inhibitor or activator compounds) for use in the present invention may also be modified in a way to form a chimeric molecule comprising a therapeutic compound fused to another, heterologous polypeptide or amino acid sequence, such as an Fc sequence for stability.

The therapeutic compound can be packaged alone or in combination with other therapeutic compounds as a kit (e.g., with one or more additional therapeutic compounds of the invention or with a statin, cholesterol lowering agents such cholestyramine and niacin, aspirin, non-steroid anti-inflammatory drugs, steroids, angiotensin converting enzyme inhibitors, platelet inhibitory agent, such as Plavix, anti-coagulative agent, such heparin, and coumadin. Additional therapeutic compounds that can be used in combination with the therapeutic compounds of the invention include compounds that inhibit smooth muscle cell proliferation or migration, including but not limited to taxol and rapamycin, and compounds that inhibit PDGF, including but not limited to Gleevec. Non-limiting examples include kits that contain, for example, two pills, a powder, a suppository and a liquid in a vial, or two topical creams.

The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

### Combination Therapies

Therapeutic compounds that inhibit the activated polypeptides of the invention can be used alone or in combination with one, two, three, four, or more of the inhibitor compounds of the invention or with a known therapeutic compound for the treatment or prevention of a vascular inflammatory disorder or an endothelial cell disorder, such as statin, cholesterol lowering agents such cholestyramine and niacin, aspirin, non-steroid anti-inflammatory drugs, steroids, angiotensin converting enzyme inhibitors, platelet inhibitory agent, such as Plavix, anti-coagulative agent, such heparin, and coumadin, compounds that inhibit smooth muscle cell proliferation or migration, such as taxol and rapamycin, and compounds that inhibit PDGF, including but not limited to Gleevec. In one example, a Tie-1 or EphA2 inhibitor compound is used in combination with a therapeutically effective amount of one, two, three, four, five, or more inhibitor compounds, where each inhibitor compound inhibits the expression level or biological activity of one or more of the following: tissue factor, thrombin, IP-10, G-SCF, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, KDR, c-RET, MER, EphA2, VEGFR2 endodomain, or Tie-2. In another example, a Tie-1 inhibitor compound is administered in combination with an eNOS activator compound. In another example, an EphA2 inhibitor compound is used in combination with a therapeutically effective amount of one, two, three, four, five, or more inhibitor compounds, where each inhibitor compound inhibits the expression level or biological activity of one or more of the following: tissue factor, thrombin, IP-10, G-SCF, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, KDR, c-RET, MER, VEGFR2 endodomain, Tie-1, or Tie-2. In addition, as EphA2 has been shown herein to function in a Src dependent manner, a Src kinase inhibitor can also be used in combination with an EphA2 inhibitor of the invention. SU5416 is one example of a Src kinase inhibitor.

Combination therapies may provide a synergistic benefit and can include sequential administration, as well as administration of these therapeutic agents, in a substantially simultaneous manner. In one example, substantially simultaneous administration is accomplished, for example, by administering to the subject a Tie-1 inhibitor compound or an EphA2 compound and a second inhibitor in multiple capsules or injections at approximately the same time. The components of the combination therapies, as noted above, can be administered by the same route or by different routes (e.g., via oral administration). In different embodiments, a first inhibitor compound (e.g., Tie-1 inhibitor or EphA2 inhibitor) may be administered by orally, while the one or more additional inhibitor compounds may be administered intramuscularly, subcutaneously, topically or all therapeutic agents may be administered orally or all therapeutic agents may be administered by intravenous injection.

### Diagnostic Methods

The polypeptides identified herein as activated or downregulated in the presence of activated Tie-1 or Tie-1 endodomain or activated thrombin can also be used for the diagnosis of vascular inflammatory disorders, such as atherosclerosis, or an endothelial cell disorder, or a risk of developing a vascular inflammatory disorder or an endothelial cell disorder. These proteins can also be used to monitor the therapeutic efficacy of compounds, including compounds of the invention, used to treat the vascular inflammatory disorder, such as atherosclerosis, or an endothelial cell disorder.

Alterations in the expression or biological activity of one or more polypeptides of the invention in a test sample as compared to a normal reference can be used to diagnose any of the vascular inflammatory disorders or endothelial cell disorders of the invention.

A subject having a vascular inflammatory disorder or an endothelial cell disorder, or a propensity to develop a vascular inflammatory disorder or an endothelial cell disorder, will show an alteration (e.g., an increase or a decrease of 10%, 20%, 30%, 40%, 50%, 60%, 70%, 80%, 90%, or more) in the expression or biological activity of one or more of the activated or downregulated polypeptides of the invention. In one example, an increase in Tie-1, Tie-1 endodomain, thrombin, VEGFR2 or VEGFR2 endodomain, EphA2, or a cytokine or tyrosine kinase shown to be upregulated in the presence of Tie-1 or thrombin expression or biological activity in a subject sample as compared to a normal reference is indicative of a vascular inflammatory disorder or a risk of developing the same. The Tie-1, Tie-1 endodomain, thrombin, VEGFR2 or VEGFR2 endodomain, EphA2, or a cytokine or tyrosine kinase shown to be upregulated in the presence of Tie-1 or thrombin (e.g., tissue factor, IP-10, G-SCF, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, KDR, c-RET, MER, VEGFR2 endodomain, or Tie-2) can include full-length polypeptide, degradation products, alternatively spliced isoforms of the polypeptide, enzymatic cleavage products of the polypeptide, the polypeptide bound to a substrate or ligand, or free (unbound) forms of the polypeptide. In one example, a decrease in the level or biological activity of eNOS in a subject sample as compared to a normal reference sample is indicative of a vascular inflammatory disorder or an endothelial cell disorder or a risk of developing the same.

Standard methods may be used to measure polypeptide levels in any bodily fluid, including, but not limited to, urine, blood, serum, plasma, saliva, or cerebrospinal fluid. Such methods include immunoassay, ELISA, Western blotting using antibodies directed to a polypeptide of the invention (e.g., including but not limited to Tie-1, Tie-1 endodomain, thrombin, VEGFR2 or VEGFR2 endodomain, EphA2, tissue factor, IP-10, G-SCF, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, c-RET, MER, or Tie-2), and quantitative enzyme immunoassay techniques. ELISA assays are the preferred method for measuring polypeptide levels. In one example, an antibody that specifically binds Tie-1, Tie-1 endodomain, thrombin, VEGFR2 or VEGFR2 endodomain, or EphA2 polypeptide is used in an immunoassay for the detection of Tie-1, Tie-1 endodomain, thrombin, VEGFR2 or VEGFR2 endodomain, or EphA2 and the diagnosis of any of the vascular inflammatory disorders or endothelial cell disorders described herein or the identification of a subject at risk of developing a vascular inflammatory disorder or an endothelial cell disorder.

The measurement of antibodies specific to a polypeptide of the invention in a subject may also be used for the diagnosis of a vascular inflammatory disorder or a propensity to develop the same. Antibodies specific to one or more polypeptides of the invention may be measured in any bodily fluid, including, but not limited to, urine, blood, serum, plasma, saliva, or cerebrospinal fluid. ELISA assays are the preferred method for measuring levels of antibodies in a bodily fluid. An increased level of, for example, anti-Tie-1, anti-Tie-1 endodomain, anti-thrombin, anti-VEGFR2 or anti-VEGFR2 endodomain, or anti-EphA2 antibodies in a bodily fluid is indicative of a vascular inflammatory disorder or an endothelial cell disorder or a propensity to develop the same.

Nucleic acid molecules encoding a polypeptide of the invention, either activated or downregulated, or fragments or oligonucleotides thereof that hybridize to a nucleic acid molecule encoding a polypeptide of the invention n at high stringency may be used as a probe to monitor expression of nucleic acid molecules encoding a polypeptide of the invention in the diagnostic methods of the invention. Any of the nucleic acid molecules above can also be used to identify subjects having a genetic variation, mutation, or polymorphism in a nucleic acid molecule that are indicative of a predisposition to develop the conditions. These polymorphisms may affect nucleic acid or polypeptide expression levels or biological activity. Detection of genetic variation, mutation, or polymorphism relative to a normal, reference sample can be used as a diagnostic indicator of a subject likely to develop a vascular inflammatory disorder or an endothelial cell disorder or a propensity to develop the same.

In one embodiment, a subject having a vascular inflammatory disorder or an endothelial cell disorder or a propensity to develop the same, will show an increase in the expression of a nucleic acid encoding a polypeptide of the invention, e.g., Tie-1, Tie-1 endodomain, thrombin, VEGFR2 or VEGFR2 endodomain, or a cytokine or tyrosine kinase shown to be upregulated in the presence of Tie-1 or thrombin (e.g., tissue factor, IP-10, G-SCF, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, c-RET, MER, or Tie-2). Methods for detecting such alterations are standard in the art and are described in Sandri et al. (Cell, 117:399-412, 2004). In one example Northern blotting or real-time PCR is used to detect mRNA levels (Sandri et al., supra, and Bdolah et al., Am. J. Physio. Regul. Integre. Comp. Physiol. 292:R971-R976, 2007).

In another embodiment, hybridization at high stringency with PCR probes that are capable of detecting a Tie-1, Tie-1 endodomain, thrombin, VEGFR2 or VEGFR2 endodomain, or a cytokine or tyrosine kinase shown to be upregulated in the presence of Tie-1 or thrombin nucleic acid molecule (e.g., tissue factor, IP-10, G-SCF, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, c-RET, MER, or Tie-2), including genomic sequences, or closely related molecules, may be used to hybridize to a nucleic acid sequence derived from a subject having a vascular inflammatory disorder, or at risk of developing such a disorder. The specificity of the probe, whether it is made from a highly specific region, e.g., the 5′ regulatory region, or from a less specific region, e.g., a conserved motif, and the stringency of the hybridization or amplification (maximal, high, intermediate, or low), determine whether the probe hybridizes to a naturally occurring sequence, allelic variants, or other related sequences. Hybridization techniques may be used to identify mutations in a nucleic acid molecule, or may be used to monitor expression levels of a gene encoding a polypeptide of the invention.

Diagnostic methods can include measurement of absolute levels of a polypeptide, nucleic acid, or antibody of the invention, or relative levels of a polypeptide, nucleic acid, or antibody of the invention as compared to a reference sample. In one example, an increase in the level or biological activity of a Tie-1, Tie-1 endodomain, thrombin, VEGFR2 or VEGFR2 endodomain, or EphA2 polypeptide, nucleic acid, or antibody as compared to a normal reference, is considered a positive indicator of a vascular inflammatory disorder or an endothelial cell disorder or a propensity to develop the same.

In any of the diagnostic methods, the level of a polypeptide, nucleic acid, or antibody, or any combination thereof, can be measured at least two different times from the same subject and an alteration in the levels (e.g., by 10%, 20%, 30%, 40%, 50%, 60%, 70%, 80%, 90%, or more) over time is used as an indicator of a vascular inflammatory disorder or an endothelial cell disorder, or the propensity to develop the same. It will be understood by the skilled artisan that for diagnostic methods that include comparing of the polypeptide, nucleic acid, or antibody level to a reference level, particularly a prior sample taken from the same subject, a change over time (e.g., an increase for Tie-1, Tie-1 endodomain, thrombin, VEGFR2 or VEGFR2 endodomain, EphA2, or a cytokine or tyrosine kinase shown to be upregulated in the presence of Tie-1 or thrombin) with respect to the baseline level can be used as a diagnostic indicator of a vascular inflammatory disorder, or a predisposition to develop the same. The level of the polypeptide (e.g., Tie-1, Tie-1 endodomain, thrombin, VEGFR2 or VEGFR2 endodomain, EphA2, or a cytokine or tyrosine kinase shown to be upregulated in the presence of Tie-1 or thrombin), nucleic acid encoding the polypeptide, or antibody that binds the polypeptide in a bodily fluid sample of a subject having a vascular inflammatory disorder, or the propensity to develop such a condition may be altered, e.g., increased by as little as 10%, 20%, 30%, or 40%, or by as much as 50%, 60%, 70%, 80%, or 90% or more, relative to the level of the polypeptide, nucleic acid, or antibody in a prior sample or samples.

The diagnostic methods described herein can be used individually or in combination with any other diagnostic method described herein for a more accurate diagnosis of the presence of, severity of, or predisposition to a vascular inflammatory disorder or an endothelial cell disorder, or a predisposition to the same. In one example, the level of two or more of the activated polypeptides of the invention (e.g., Tie-1, Tie-1 endodomain, thrombin, VEGFR2 or VEGFR2 endodomain, EphA2, tissue factor, G-CSF, IL-6, IP-10, VCAM-1, ICAM-1, CCL20, CCL2, CXCL5, E-selectin, soluble CD44, EGFR, insulin receptor, IGF-1R, AXL, HGFR, FLt-1, c-RET, MER, and Tie-2) is measured. In another example, the level of eNOS is also measured, wherein a decrease in the level of eNOS as compared to a reference sample is diagnostic of a vascular inflammatory disorder or an endothelial cell disorder or a propensity to develop the same.

### Diagnostic Kits

The invention also provides for a diagnostic test kit. For example, a diagnostic test kit can include polypeptides (e.g., antibodies that specifically bind to any of the polypeptides of the invention), and components for detecting, and more preferably evaluating binding between the polypeptide (e.g., antibody) and the polypeptide of the invention. In another example, the kit can include a polypeptide of the invention, or fragment thereof, for the detection of antibodies in the serum or blood of a subject sample that bind to polypeptides of the invention. For detection, either the antibody or the polypeptide is labeled, and either the antibody or the polypeptide is substrate-bound, such that the polypeptide-antibody interaction can be established by determining the amount of label attached to the substrate following binding between the antibody and the polypeptide. A conventional ELISA is a common, art-known method for detecting antibody-substrate interaction and can be provided with the kit of the invention. The polypeptides of the invention can be detected in virtually any bodily fluid, such as urine, plasma, blood serum, semen, or cerebrospinal fluid. A kit that determines an alteration in the level of a polypeptide of the invention relative to a reference, such as the level present in a normal control, is useful as a diagnostic kit in the methods of the invention. Such a kit may further include a reference sample or standard curve indicative of a positive reference or a normal control reference.

Desirably, the kit will contain instructions for the use of the kit. In one example, the kit contains instructions for the use of the kit for the diagnosis of a vascular inflammatory disorder or an endothelial cell disorder or a propensity to develop the same. In yet another example, the kit contains instructions for the use of the kit to monitor therapeutic treatment or dosage regimens.

### Subject Monitoring

The diagnostic methods described herein can also be used to monitor a vascular inflammatory disorder or an endothelial cell disorder during therapy or to determine the dosages of therapeutic compounds. For example, alterations (e.g., a decrease as compared to the positive reference sample or level for a vascular inflammatory disorder or an endothelial cell disorder indicates an improvement in or the absence of vascular inflammatory disorder or an endothelial cell disorder). In this embodiment, the levels of the polypeptide, nucleic acid, or antibodies are measured repeatedly as a method of not only diagnosing disease but also monitoring the treatment, prevention, or management of the disease. In order to monitor the progression of a vascular inflammatory disorder or an endothelial cell disorder in a subject, subject samples are compared to reference samples taken early in the diagnosis of the disorder. Such monitoring may be useful, for example, in assessing the efficacy of a particular drug in a subject, determining dosages, or in assessing disease progression or status. For example, levels of Tie-1, Tie-1 endodomain, thrombin, VEGFR2 or VEGFR2 endodomain, EphA2, tissue factor, G-CSF, IL-6, IP-10, VCAM-1, ICAM-1, CCL20, CCL2, CXCL5, E-selectin, soluble CD44, EGFR, insulin receptor, IGF-1R, AXL, HGFR, FLt-1, c-RET, MER, or Tie-2, or any combination thereof, can be monitored in a patient having a vascular inflammatory disorder or an endothelial cell disorder and as the levels of decrease, the dosage or administration of therapeutic inhibitor compounds may be decreased as well. In addition, the diagnostic methods of the invention can be used to monitor a subject that has risk factors indicative of a vascular inflammatory disorder or an endothelial cell disorder (e.g., a subject having a family history of a cardiovascular disease or a history of pre-eclampsia or eclampsia). In such an example, the therapeutic methods of the invention or those known in the art can then be used proactively to promote endothelial cell health and to prevent the disorder from developing or from developing further.

### VEGFR-2 Compounds

VEGFR-2 was identified as one of the tyrosine kinases that was activated by thrombin stimulation of endothelial cells in our assays. VEGFR-2, was activated in a VEGF-independent manner and a previously unidentified truncated form of VEGFR-2 was also identified. We have shown that this newly discovered truncated form, which we termed the VEGFR2 endodomain, results from receptor cleavage and shedding of the VEGFR-2 ectodomain. The VEGFR2 endodomain has a molecular weight of approximately 120 kDa (but can be 90 kDa, 100 kDa, 105 kDa, 110 kDa, 115 kDa, 120 kDa, 125 kDa, 130 kDa, 135 kDa, 140 kDa, 145 kDa, and 150 kDa depending on the conditions used for determining the molecular weight) is detected by antibodies that specifically bind to the carboxy terminus of VEGFR2, and is phosphorylated in its activated form.

The invention features compositions that include an isolated or purified VEGFR2 endodomain, including the active phosphorylated form. The compositions can be a VEGFR2 endodomain fusion protein where the VEGFR2 endodomain is fused to another polypeptide, such as an Fc fusion, to increase stability of the protein or a tag polypeptide sequence for detection.

The invention also provides a composition that includes a biologically active VEGFR2 endodomain and a pharmaceutically acceptable carrier, examples of which are described above. Pharmaceutical compositions useful for promotion of vascular or lymph endothelial cell growth generally include a therapeutically effective amount of the VEGFR2 endodomain in a pharmaceutically acceptable carrier. Optionally, the pharmaceutical compositions can further include another cell growth factor such as VEGF and/or PDGF, or fragments thereof.

Because the VEGFR2 endodomain is an activated form of VEGFR2, the invention also features the use of the VEGFR2 endodomain to promote any of the functions that VEGF is known to promote through the VEGFR2, including but not limited to angiogenesis, vasculogenesis, pseudovasculogenesis, vessel co-option, survival of endothelial cells, proliferation of endothelial cells, migration of endothelial cells, endothelial permeability, and inflammation. Furthermore, the invention features the use of the VEGFR2 endodomain, or an activated form thereof, for the treatment of any disorder in which VEGF, VEGFR2, or agonists thereof would be useful. Examples include any disorder that is characterized by insufficient angiogenesis, vasculogenesis, insufficient vessel regression, altered vasomotor tone, hypercoagulation, anti-inflammatory properties, and poor endothelial cell health. Non-limiting examples include Alzheimer's disease, amyotrophic lateral sclerosis, diabetic neuropathy, stroke, diabetes, restenosis, coronary artery disease, peripheral vascular disease, vasculitis, vasculitidis, injuries or wounds of the blood vessels or heart, Wegner's disease, gastric or oral ulcerations, cirrhosis, hepatorenal syndrome, Crohn's disease, hair loss, skin purpura, telangiectasia, venous lake formation, delayed wound healing, pre-eclampsia, eclampsia, ischemia-reperfusion injury, acute renal failure, hypertension, chronic or acute infection, menorrhagia, neonatal respiratory distress, pulmonary fibrosis, emphysema, nephropathy, hemolytic uremic syndrome, glomerulonephritis, sclerodoma, and vascular abnormalities. Additional conditions that can be treated using the VEGFR2 endodomain, or the activated form thereof, include dermal ulcers, including the categories of pressure sores, venous ulcers, and diabetic ulcers, as well as full-thickness burns and injuries where angiogenesis is required to prepare the burn or injured site for a skin graft or flap. In this case, the VEGFR2 endodomain, or the activated form thereof, is either applied directly to the site or it is used to soak the skin or flap that is being transplanted prior to grafting. In a similar fashion, the VEGFR2 endodomain, or the activated form thereof, can be used in plastic surgery when reconstruction is required following a burn or other trauma, or for cosmetic purposes.

For the traumatic indications referred to above, the VEGFR2 endodomain, or the activated form thereof, will be formulated and dosed in a fashion consistent with good medical practice taking into account the specific disorder to be treated, the condition of the individual patient, the site of delivery of the VEGFR2 endodomain, or the activated form thereof, the method of administration, and other factors known to practitioners.

In cases where the VEGFR2 endodomain, or the activated form thereof, is being used for topical wound healing, as described above, it may be administered by any of the routes described below for the re-endothelialization of vascular tissue, or more preferably by topical means. In these cases, it will be administered as either a solution, spray, gel, cream, ointment, or dry powder directly to the site of injury. Slow-release devices directing the VEGFR2 endodomain, or the activated form thereof, to the injured site will also be used. In topical applications, the VEGFR2 endodomain, or an activated form thereof, will be applied either in a single application, or in dosing regimens that are daily or every few days for a period of one week to several weeks.

The VEGFR2 endodomain, or an activated form thereof, can be used as a post-operative wound healing agent in balloon angioplasty, a procedure in which vascular endothelial cells are removed or damaged, together with compression of atherosclerotic plaques. The VEGFR2 endodomain, or the activated form thereof, can be applied to inner vascular surfaces by systemic or local intravenous application either as intravenous bolus injection or infusions. If desired, the VEGFR2 endodomain, or an activated form thereof, can be administered over time using a micrometering pump. Suitable compositions for intravenous administration comprise the VEGFR2 endodomain, or an activated form thereof, in an amount effective to promote endothelial cell growth and a parenteral carrier material. The VEGFR2 endodomain, or an activated form thereof, can be present in the composition over a wide range of concentrations, for example, from about 50 μg/mL to about 1,000 μg/mL using injections of 3 to 10 mL per patient, administered once or in dosing regimens that allow for multiple applications. Any of the known parenteral carrier vehicles can be used, such as normal saline or 5-10% dextrose. Therapeutic formulations of VEGFR2 endodomain, or an activated form thereof, are prepared for storage by mixing VRP having the desired degree of purity with optional physiologically acceptable carriers, excipients, or stabilizers (Remington's Pharmaceutical Sciences, (20^{th} edition), ed. A. Gennaro, 2000, Lippincott, Williams & Wilkins, Philadelphia, Pa.) in the form of lyophilized cake or aqueous solutions. Acceptable carriers, excipients, or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counter-ions such as sodium; and/or non-ionic surfactants such as Tween, Pluronics or polyethylene glycol (PEG).

The invention also features compositions that include inhibitor compounds that specifically inhibit or reduce the biological activity or expression of the VEGFR2 endodomain, including the active phosphorylated form. The inhibitor compound can be any compound (peptidyl or non-peptidyl), small molecules, nucleic acids, or otherwise. Pharmaceutical compositions of the invention also include antagonists that specifically inhibit or reduce the biological activity or expression of the VEGFR2 endodomain, including the active phosphorylated form. The antagonists can include compounds (peptidyl or non-peptidyl), small molecules, antibodies, nucleic acids, or otherwise. In one example, the inhibitor compound is an antagonistic antibody or polypeptide that specifically binds to the VEGFR2 endodomain and not the full-length VEGFR2. In another example, the inhibitor compound is a small molecule inhibitor that binds to the ATP binding pocket of VEGFR2 (e.g., SU5416 or derivates or analogs thereof). The inhibitor compound can further include a pharmaceutically acceptable carrier. Such antagonistic compositions are useful for reducing or inhibiting angiogenesis, vasculogenesis, pseudovasculogenesis, vessel co-option, survival of endothelial cells, proliferation of endothelial cells, migration of endothelial cells, endothelial permeability, and inflammation. In one embodiment, a VEGFR2 endodomain specific inhibitor can be used to treat or prevent any of the following angiogenic disorders: cancers which require neovascularization to support tumor growth, infectious diseases, autoimmune disorders, vascular malformations, DiGeorge syndrome, HHT, cavernous hemangioma, transplant arteriopathy, vascular access stenosis associated with hemodialysis, vasculitis, vasculitidis, vascular inflammatory disorders, atherosclerosis, obesity, psoriasis, warts, allergic dermatitis, scar keloids, pyogenic granulomas, blistering disease, Kaposi sarcoma, persistent hyperplastic vitreous syndrome, retinopathy of prematurity, choroidal neovascularization, macular degeneration, diabetic retinopathy, ocular neovascularization, primary pulmonary hypertension, asthma, nasal polyps, inflammatory bowel and periodontal disease, ascites, peritoneal adhesions, contraception, endometriosis, uterine bleeding, ovarian cysts, ovarian hyperstimulation, arthritis, rheumatoid arthritis, chronic articular rheumatism, synovitis, osteoarthritis, osteomyelitis, osteophyte formation, sepsis, and vascular leak.

### EphA2

EphA2 was identified as one of the tyrosine kinases that was activated by thrombin stimulation of endothelial cells in our assays. This activation is rapid and appears to be independent of EphA2 cognate ligands including Ephrin A1. Functionally, we have discovered that EphA2 is an absolute requirement for thrombin-induced ICAM-1 upregulation in endothelial cells and that this upregulation occurs in NFkB dependent manner. We have also discovered that EphA2 knockdown potently reduces leukocyte attachment to thrombin-stimulated endothelial cells in vitro. Ephrins and Eph receptors have been implicated to be important in inflammation. For example, Ephrin-A1 was first identified as an immediately-early response gene of endothelial cells induced by inflammatory stimuli such as TNF-a, IL-1f3, and lipopolysaccharide (Dixit, Green et al., J. Biol. Chem. 265: 2973-2978, (1990); Holzman, Marks et al., Mol. Cell. Biol. 10: 5830-5838, (1990)). Ephrin receptors, including EphA2, are shown to be upregulated during inflammation (Ivanov, Steiner et al., Physiol. Genomics 21: 152-160, (2005)). In addition, EphB/EphrinB system appears to play a role in the inflammatory responses in rheumatoid arthritis (Kitamura, Kabuyama et al., Am J Physiol Cell Physiol (2007)). Other than attribution of EphA2 being a mediator of TNF-a-induced angiogenesis in micro-pocket corneal assays in mice (Pandey, Shao et al., Science (New York, N.Y. 268: 567-569, (1995)), very little is known about the specific functions of these Eph receptors/Ephrins in endothelial inflammation. Importantly, our observation that EphA2 is a downstream mediator of thrombin in regulation of ICAM-1 expression provides the first direct evidence to link EphA2 to thrombin-endothelial biology. In addition, it is worth noting that in our experiments, EphB/Ephrin B, previously believed to have a role in inflammation, was not activated upon thrombin stimulation of endothelial cells.

The invention features inhibitor compounds that specifically inhibit or reduce the biological activity or expression of EphA2, including the active phosphorylated form. Such inhibitor compounds can be used to treat vascular inflammatory disorders and to inhibit thrombin activation of pro-inflammatory pathways. Desirably, the EphA2 inhibitor compound will inhibit the pro-inflammatory activity of thrombin in the absence of inhibition of the pro-coagulation activity of thrombin. EphA2 inhibitor compounds can include any compound (peptidyl or non-peptidyl), small molecules, nucleic acids, or otherwise. In one example, the inhibitor compound is an antagonistic antibody or polypeptide that specifically binds to the EphA2 and that reduces or prevents the biological activity of EphA2. In another example, the inhibitor compound is a small molecule inhibitor that binds to the ATP binding pocket of EphA2 or to the substrate binding domain of EphA2. The EphA2 inhibitor compound can also be a nucleic acid molecule that reduces or inhibits the expression of EphA2 polypeptide or nucleic acid molecules and examples of such siRNA molecules are provided in the Examples section below.

For any of the EphA2 inhibitor compounds, a reduction in the biological activity of EphA2 can be evaluated using any of the assays described below including, but not limited to, assays for a reduction in EphA2 protein expression levels, kinase assays, ICAM-1 activation assays, NFkB assays, leukocyte attachment assays, and assays for binding to substrates including CrkL, α and β subunits of PI3K, and SHP-2.

For any of the EphA2 inhibitor compounds, the compounds can be in a composition that can further include a pharmaceutically acceptable carrier. The composition can be formulated in any formulation as described above. Such antagonistic compositions are useful for reducing or inhibiting angiogenesis, vasculogenesis, pseudovasculogenesis, vessel co-option, survival of endothelial cells, proliferation of endothelial cells, migration of endothelial cells, endothelial permeability, and inflammation. Desirably, the EphA2 inhibitor compound is used to treat or prevent an endothelial cell disorder or a vascular inflammatory disorder, such as atherosclerosis.

In one specific example, an EphA2 inhibitor compound can be used to treat or prevent pre-eclampsia or eclampsia. Pre-eclampsia is characterized by an anti-angiogenic state and a pro-inflammatory state. Inhibitors of EphA2 would be effective for the treatment or prevention of pre-eclampsia or eclampsia, particularly the inflammatory aspects of the disorder.

### Screening Assays

As discussed above, we have discovered that Tie-1 expression upregulates thrombin and a number of cytokine and tyrosine kinase molecules that are involved in endothelial cell dysfunction and vascular inflammatory disorders. Based on these discoveries, Tie-1, Tie-1 endodomain, VEGFR2, VEGFR2 endodomain, EphA2, or thrombin are useful for the high-throughput low-cost screening of candidate compounds to identify those that modulate, alter, or decrease (e.g., by at least 10%, 20%, 30%, 40%, 50%, 60%, 70%, 80%, 90%, 95%, or more), the expression or biological activity of Tie-1. Tie-1 endodomain, thrombin, or any of the polypeptides shown to be up or down regulated by the expression of activated forms of these proteins. Compounds that decrease the expression or biological activity of an activated polypeptide of the invention (e.g., Tie-1, Tie-1 endodomain, thrombin, VEGFR2 or VEGFR2 endodomain, EphA2, tissue factor, G-CSF, IL-6, IP-10, VCAM-1, ICAM-1, CCL20, CCL2, CXCL5, E-selectin, soluble CD44, EGFR, insulin receptor, IGF-1R, AXL, HGFR, FLt-1, c-RET, MER, and Tie-2) can be used for the treatment or prevention of a vascular inflammatory disorder or endothelial cell disorder. Compounds that increase the expression or biological activity of a downregulated polypeptide of the invention (e.g., eNOS) can also be used for the treatment or prevention of a vascular inflammatory disorder or endothelial cell disorder. Candidate compounds can be tested for their effect on thrombin or Tie-1 biological activities (e.g., phosphorylation of proteins including MLC, VE cadherin, and p120; increased endothelial cell permeability; intracellular gap junction formation) using assays known in the art or described in the Examples below.

In general, candidate compounds are identified from large libraries of both natural product or synthetic (or semi-synthetic) extracts, chemical libraries, or from polypeptide or nucleic acid libraries, according to methods known in the art. Those skilled in the field of drug discovery and development will understand that the precise source of test extracts or compounds is not critical to the screening procedure(s) of the invention.

### EXAMPLES

### Example 1

### Upregulation of Proinflammatory Cytokines and Adhesion Molecules in Endothelial Cells by Overexpression of Tie-1 Endodomain

Tie-1 receptor is an endothelial specific cell surface tyrosine kinase. Genetic deletion of this protein in mice confers embryonic lethality between days 13.5 to 14.5 of gestation. In murine embryonic development, Tie-1 appears not to be required in early angiogenic processes but is important in maintaining vessel integrity. In agreement with these findings, expression knockdown of Tie-1 in zebrafish by antisense morpholino oligonucleotides does not appear to affect vessel development and integrity up to day 3 post fertilization, a period when the basic framework of the vasculature is established to support initial blood flow. However, vessels begin to regress from this point onwards. Previously patent lumens, especially the caudal artery and vein, regress and narrow, resulting in sluggish blood flow.

Since a high affinity binding, signaling ligand has not been conclusively identified for Tie-1, very little is known about the specific biology of this molecule. There have even been conflicting reports regarding the kinase activity of Tie-1. For example, the function of Tie-1 was investigated by Kontos at el. (Mol. Cell. Bio. 22: 1704-1713, (2002)) using a chimeric construct composed of the extracellular domain of c-fms receptor and the intracellular domain of Tie-1. When expressed in NIH3T3 cells, this chimera was found to be tyrosine phosphorylated in response to CSF-1, resulting in the activation of the PI3 kinase and AKT pathways. As a result, UV-irradiation-induced apoptosis was blocked. However, a different conclusion was reached from an analogous study. Marron et al. constructed a chimera composed of the extracellular domain of the nerve growth factor receptor TrkA and the C terminal domain of Tie-1 (Marron et al., J. Biol. Chem. 275: 39741-39746, (2000)). When expressed in bovine aortic endothelial cells, no autophosphorylation was detected on this chimeric receptor when stimulated with nerve growth factor. Recently, Saharinen et al. reported a study suggesting the angiopoietin 1 and 4 could induce Tie-1 autophosphorylation in vitro in a largely Tie-2 dependent manner (Saharinen et al., J. Cell Biol. 169: 239-243, (2005)). However, biological functions of such activation were not addressed. In addition, an in vivo tumor experiment further illustrates the complexity and our lack of knowledge of the mechanism of Tie-1 activation. Tie-1 has been shown to be upregulated in the vasculature in tumors, yet overexpression of the soluble extracellular domain of Tie-1 by tumors does not affect tumor growth in mice.

Some receptor tyrosine kinases can be activated by a ligand independent mechanism that involves shedding of the ectodomain. Examples include Her2/Neu receptor, ErbB-4, the sevenless receptor tyrosine kinase in drosophila, TrkA receptor, and insulin receptor. Consistent with this theme, a mutant form of EGF receptor commonly found in tumors has an in-frame deletion of the EGF-ligand binding domain and remains constitutively active. This raises the possibility that Tie-1 receptor could be activated through a ligand independent mechanism.

In vitro experiments have shown that Tie-1 undergoes ectodomain shedding upon stimulation to generate a membrane-bound C-terminal endodomain. External stimuli that can result in Tie-1 cleavage include phorbol ester, VEGF, thrombin, TNFα, and LPS. This shedding event appears to be dependent on a cell-surface bound metalloproteinase. In addition, as noted above, change in shear stress has also been shown to induce Tie-1 ectodomain shedding. However, there has been no report to date to show that the endodomain of Tie-1 is either phosphorylated or has kinase activity. Tie-1 endodomain has been shown to co-immunoprecipitate with a tyrosine-phosphorylated protein later identified to be SHP2. Therefore, Tie-1 endodomain generated by ectodomain shedding may be capable of transmitting intracellular signals.

Expression of Tie-1 in adult vasculature may be a marker of perturbed flow experienced by endothelial cells. For example, Tie-1 promoter activity is asymmetrically upregulated in aortic valve endothelial cells at locations where disturbed blood flow is expected to be high. Tie-1 is also upregulated in lesions of arteriovenous malformations, a location where hemodynamic stress due to increased flow and pressure resulted from arteriovenous shunting is expected to be high. In addition, Tie-1 promoter activity in mice is specifically upregulated in endothelial cells at arterial bifurcations and in endothelial cells at the branch points of arterioles and capillaries. The arterial sites are well known to be atherosclerosis-prone. Strikingly, endogenous Tie-1 mRNA is upregulated in atherosclerotic lesions in ApoE-null mice and in endothelial cells in the vicinity of abdominal aneurysms and vein-to-artery interposition grafts. Despite its unique spatial expression pattern in the vasculature, Tie-1 has not been studied in the context of vascular dysfunctions associated with hemodynamic stress, such as atherosclerosis, to date, an issue that is addressed in the Examples described herein.

Thrombin may also play a role in the development of atherosclerosis. Studies show that inhibition of thrombin with specific inhibitors, hirudin for example, reduces the development of stenosis after balloon angioplasty in rabbit, rat, and pig models. Furthermore, the thrombin receptor PAR-1 has been shown to be upregulated in human atherosclerotic plaques and vascular lesions. Additionally, tissue factor is upregulated in human atherosclerotic lesions. Interestingly, tissue factor expression and activity are upregulated in vitro when endothelial cells are exposed to oscillatory shear stress. Consistent with these observations, active thrombin is present in human atherosclerotic intima. In addition, plasminogen activator inhibitor-1 (PAI-1) null mice showed significantly reduced atherosclerosis development at the carotid bifurcations but not in the aortic arch. Since a deficiency of PAI-1 should result in an increase in fibrin clearance by plasminogen activators, the results of this study suggest that there is enhanced thrombin activity and fibrin deposition at arterial bifurcations. Perhaps, upregulation of thrombin activity may also be a result of turbulent flow. However, thrombin affects many cell types. Information on the contribution of the endothelial component of thrombin activation to atherosclerosis is very limited. Furthermore, a relationship between thrombin and Tie-1 has never been established.

To date, little is known about the molecular mechanisms that govern the initiation of atherosclerosis at specific sites. ICAM-1, VCAM-1, and nitric oxide have all been implicated.

Knowing that turbulent flow may cleave Tie-1, we hypothesized that this cleavage may initiate inflammation. We, thus, tested whether overexpression of Tie-1 endodomain would upregulate the secretion of proinflammatory cytokines by endothelial cells. Expression of inflammatory markers ICAM-1 and VCAM-1 in endothelial cells was also examined.

To facilitate in vitro analysis, overexpression was achieved through either retroviral or adenoviral infection. Therefore, two different routes of overexpression (stable and transient) were provided to corroborate the findings. Initially, zebrafish Tie-1 endodomain was used for stable expression. The endodomain of zebrafish Tie-1 has a high protein sequence identity to human (>87%) and a low GC content in the coding sequence (−46%). The mouse Tie-1 endodomain was also cloned and used in subsequent experiments (protein sequence identity to human: 96%; coding region GC content: 57%).

Methods: Human pulmonary artery endothelial (HPAE) cells stably expressing zebrafish Tie-1 endodomain or GFP via retroviral infection were grown to confluency. Cytokine contents in the conditioned media were screened using the TranSignal Angiogenesis Antibody Array (Panomics). Expression of the candidate gene was verified by ELISA (R&D) and real-time PCR (Taqman, ABI). Results were further validated by infecting both HPAE and HUVE cells with adenovirus encoding mouse Tie-1 endodomain (MOI˜10). Expression of candidate genes was again verified by real-time PCR. All real-time PCR experiments were normalized to GAPDH mRNA content and analyzed as reported (Dupuy et al., Exp. Cell Res. 185: 363-372, (1989)).

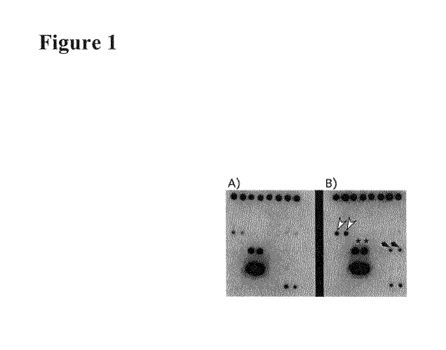

Results: Using an antibody array, we detected upregulation of three cytokine inflammatory markers in HPAE cells when Tie-1 endodomain was stably overexpressed (FIGS. 1A and 1B). They were interferon-inducible protein-10 (IP-10; solid arrows), granulocyte-colony stimulating factor (G-CSF; open arrows), and interleukin-6 (IL-6, asterisks). Other factors screened by this experiment that did not show a significant change at the protein level included angiotensin, IL-1α, FGFα, IFNγ, IL-1β, FGFβ, IL-12, HGF, TNFα, leptin, IL-8, TGFβ, TIMP1, TIMP2, P1GF, and VEGF. Upregulation of these three cytokines by Tie-1 endodomain expression is particularly relevant to atherosclerosis development. For example, injections of exogenous IL-6 significantly enhanced early development of atherosclerosis in both C57B1/6 mice and an atherosclerosis-prone mouse line (ApoE null) (Huber et al., Arterioscler. Thromb. Vasc. Biol. 19: 2364-2367, (1999)). Moreover, G-CSF is a chemotactic agent and a growth stimulant for vascular smooth muscle cells (Chen et al., Proc Soc Exp Biol Med 196: 280-283, (1991); Chen et al., Arterioscler. Thromb. Vasc. Biol. 24: 1217-1222, (2004)) and can induce expression of E-Selectin, VCAM-1, and ICAM-1 in endothelial cells, resulting in enhanced leukocyte adhesion to endothelial monolayer in vitro (Fuste et al., Haematologica 89: 578-585, (2004)). IP-10 also plays a role in atherosclerosis. It has been shown to be upregulated in endothelial cells in atherosclerotic lesions (Mach et al., J. Clin. Invest. 104: 1041-1050, (1999)) and is a chemotactic and mitogenic factor for smooth muscle cells (Wang et al., J. Biol. Chem. 271: 24286-24293, (1996)). In addition, IP-10 is also a potent chemoattractant for monocytes and activated T-lymphocytes (Taub et al., J. Exp. Med. 177: 1809-1814, (1993); Farber, J Leukoc Biol 61: 246-257, (1997)). Importantly, atherosclerosis development was significantly inhibited in IP10^{-/-}/ApoE^{-/-} double knockouts compared to control ApoE^{-/-} mice (Heller et al., Circulation 113: 2301-2312, (2006)).

We performed a cDNA Microarray experiment by infecting HUVECs with an adenovirus expressing either GFP or Tie-1. At 24 and 48 hours post infectin, total RNAs were harvested using the Rneasy Kit (Qiagen). The RNA samples were submitted to the BIDMC for analysis using the Human Genome U133 Plus genechip from Affymetrix. The results of this analysis are described herein and in the Appendix.

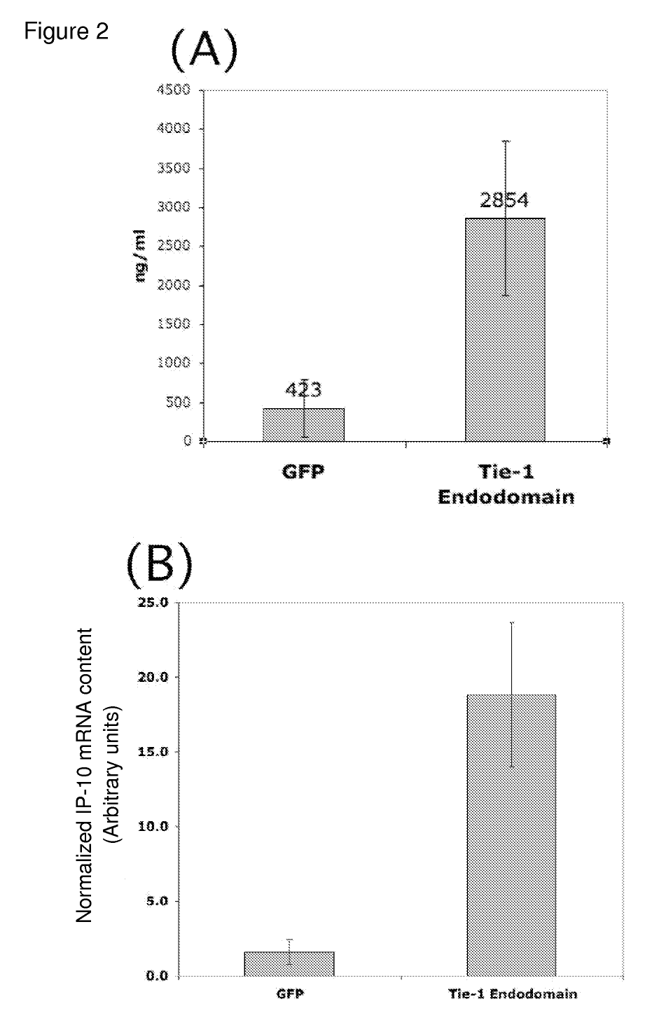

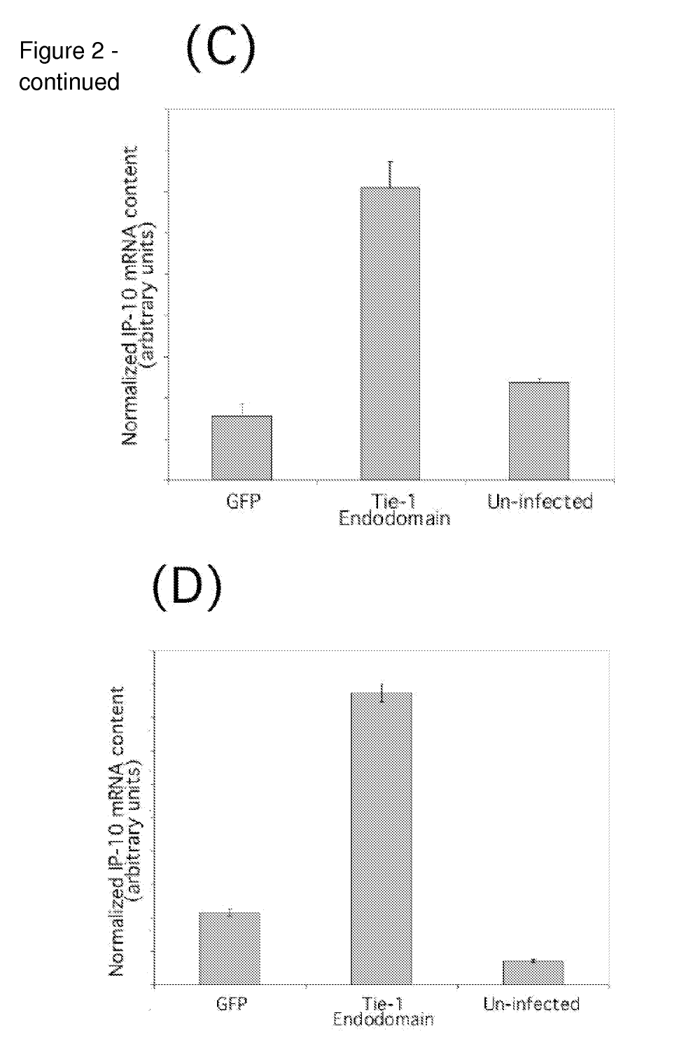

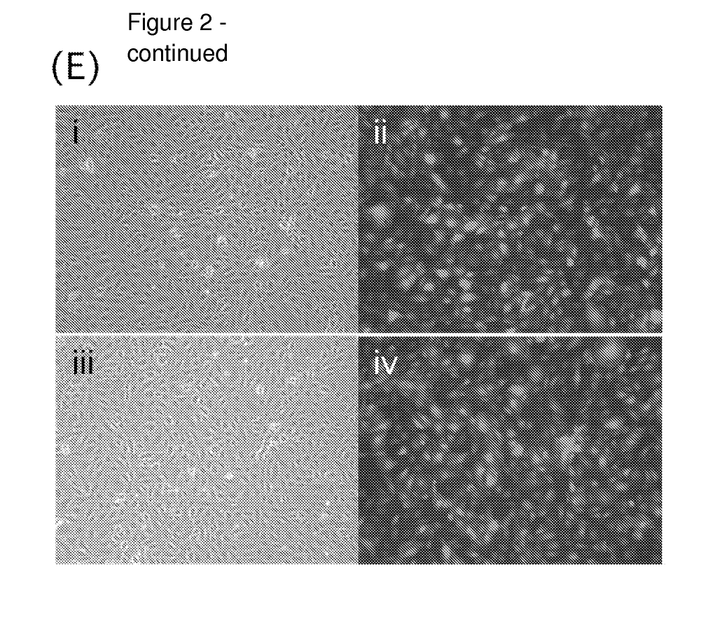

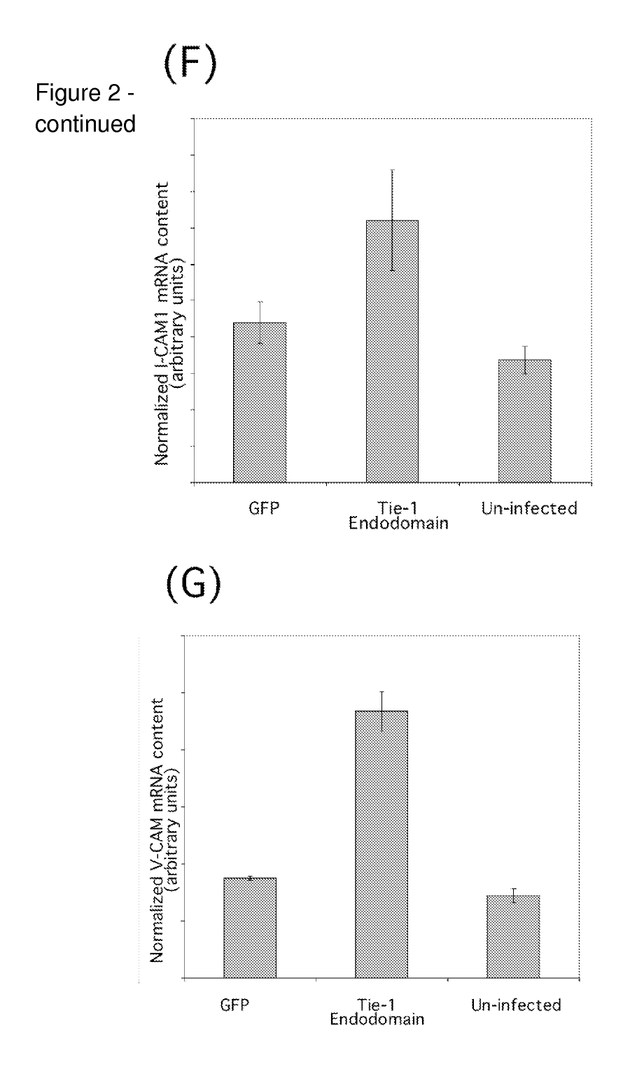

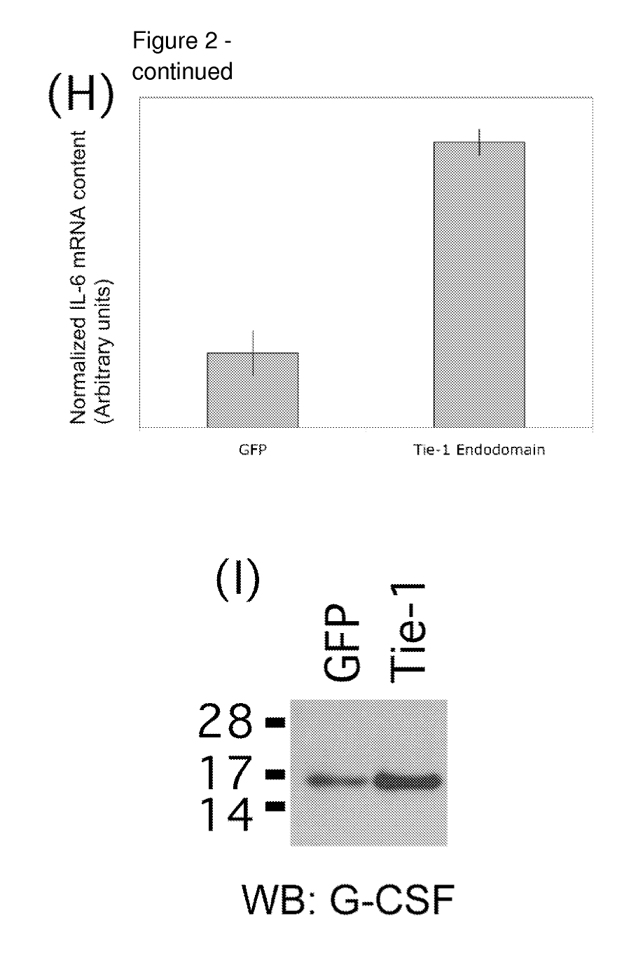

Next, we sought to validate these findings of proinflammatory cytokine upregulation induced by Tie-1 endodomain. First, upregulation of IP-10 in HPAE cells stably expressing Tie-1 endodomain was verified at the protein level by ELISA (FIG. 2A) and at the mRNA level by real-time PCR (FIG. 2B). Transient overexpression of mouse Tie-1 endodomain via adenovirus also caused upregulation of IP-10 at the mRNA level in both HPAE and HUVE cells (FIGS. 2C and 2D, respectively), recapitulating the results obtained from stable-HPAE cells. A control experiment using uninfected endothelial cells was carried out at the same time to show that the upregulation of expression was not due to viral infection. At the MOI chosen (˜10), adenoviral infection did not appear to affect the health of endothelial cells, as judged by morphology (FIG. 2E). Since expression of ICAM-1 and VCAM-1 has been reported to be upregulated at atherosclerosis-prone sites in mice (Nakashima et al., Arterioscler. Thromb. Vasc. Biol. 18: 842-851, (1998)), the effect of Tie-1 endodomain on ICAM-1 and VCAM-1 expression was also examined. By real-time PCR, a significant induction of ICAM-1 and VCAM-1 was observed in HUVE cells (FIGS. 2 F and 2G, respectively) in response to Tie-1 endodomain expression. Similar results were obtained in HPAE cells. Using this transient expression assay, we also validated that IL-6 (FIG. 2H) and G-CSF (FIG. 2I) were upregulated when Tie-1 endodomain was overexpressed in HUVECs. From these results, we conclude that overexpression of Tie-1 endodomain in endothelial cells elicits a proinflammatory response, as judged by the upregulation of IP-10, IL-6, G-CSF, ICAM-1, and VCAM-1.

### Example 2

### Expression of Tie-1 Endodomain in Endothelial Cells Enhances Attachment of Monocytic Cell Line U937

ICAM-1 and VCAM-1 are both upregulated in endothelial cells in response to Tie-1 endodomain overexpression (FIGS. 2F and 2G). Since both adhesion molecules are important in leukocyte binding to the endothelium, we performed cell adhesion assays to test whether retention of cells of monocytic lineage on HUVEC would be affected by Tie-1 endodomain.

Methods: A published procedure was followed with modifications (Kalogeris et al., Am J Physiol 276: C856-864, (1999)). 300,000 HUVE cells were seeded in each well of a 6-well plate and infected with either GFP— or Tiel endodomain-adenovirus. Medium was changed five hours post infection. Adhesion assays were performed 24 hours later. U937 (ATCC) cells were first labeled with a red fluorescent dye using Cell Tracker Red CMTPX (Molecular Probes). 1×10⁶ labeled U937 cells were resuspended in 0.5 ml endothelial medium and added to HUVE cells. After incubation at 37° C. for 1 hr, unattached U937 cells were washed away gently with endothelial medium five times. Attached U937 cells were visualized using fluorescence microscopy.

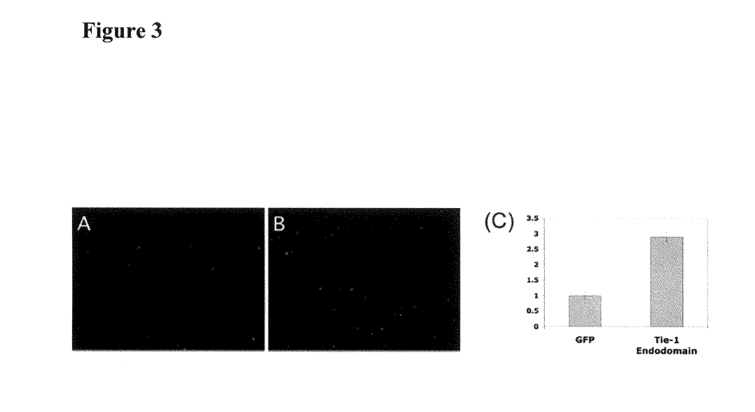

Results: Using an in vitro attachment assay, we showed that adhesion of U937 cells to HUVE cells was significantly enhanced by the expression of Tie-1 endodomain (FIGS. 3A-3C). This is consistent with the observation described in Example 1 that both ICAM-1 and VCAM-1 were upregulated in the presence of Tie-1 endodomain.

### Example 3

### Expression of Tie-1 Endodomain in Endothelial Cells Stimulates Migration of Smooth Muscle Cells

Activation of smooth muscle cells is an essential step in the development of atherosclerotic lesions. Since IP-10 and G-CSF are potent chemotactic agents for smooth muscle cells and we have shown that they are upregulated when Tie-1 endodomain is expressed (FIG. 2A-2I), we tested whether the conditioned medium from HUVE cells expressing Tie-1 endodomain would promote smooth muscle cell migration in vitro.

Methods: HUVECs were infected with control GFP or Tie-1 endodomain adenovirus as described above. Conditioned media were collected 48 hrs post infection, and cell debris was removed by centrifugation. The Transwell system with pore size of 5 μm in a 24-well format (Corning) was used in the migration assays. Human pulmonary artery smooth muscle cells (HPASMC, Cambrix) were seeded in the insert in 100 μl of smooth muscle cell medium with 0.5% FBS. HUVEC conditioned medium (600 μl) was placed in the lower chamber. Eight hours later, cells were stained with Cell Tracker Red CMTPX (Molecular Probes). HPASMCs that had not migrated were removed by a cotton swab. Migrated, stained cells were fixed in 4% PFA in PBS for 5 mins and visualized by fluorescence microscopy.

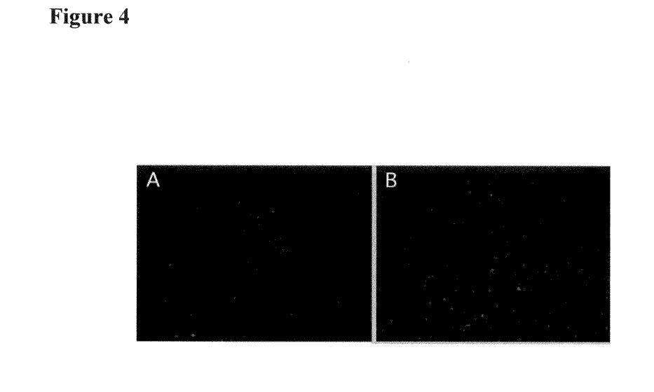

Results: As shown in FIGS. 4A-4B, conditioned medium from HUVECs expressing Tie-1 endodomain significantly stimulated migration of HPASMCs. This is consistent with our observation of IP-10 and G-CSF upregulation and that both stimulate chemotaxis of smooth muscle cells.

### Example 4

### Tie-1 Endodomain Expression Activates p38 MAP Kinase

We have shown that expression of Tie-1 endodomain upregulates several proinflammatory cytokines, VCAM-1, and ICAM-1. We investigated the intracellular signaling pathway responsible for such upregulation by probing MAP kinase p38 activation, since it has been reported that the p38 pathway is critical for inducible expression of IP-10, VCAM-1, and ICAM-1.

Methods: HUVE cells were infected with either GFP- or Tie-1 endodomain adenovirus as described above. Culture medium was changed 5 hours after infection. Activation of p38 was determined 48 hrs post infection by Western blot analysis using a phospho-specific anti p38 antibody (EMD Biosciences). To control for loading, the PVDF membrane was stripped and reblotted with an antibody against p38 cc (EMD Biosciences).

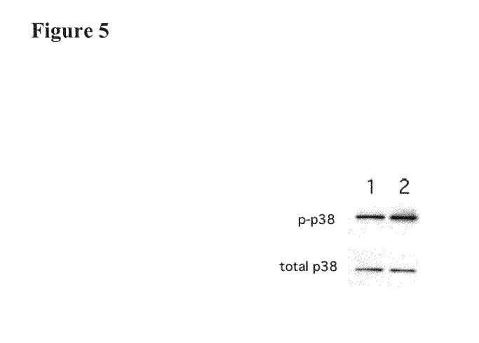

Results: As shown in FIG. 5, the basal activation level of p38 in HUVE cells expressing Tie-1 endodomain was elevated (FIG. 5 lane 2), when compared to that in cells expressing only GFP (Figure lane 1). This observation is consistent with published reports showing that activation of p38 is necessary for induction of inflammatory molecules such as IP-10, VCAM-1, and ICAM-1. Therefore, p38 activation may be central to the proinflammatory response induced by Tie-1 endodomain in endothelial cells.

### Example 5

### Tie-1 Endodomain Expression in Endothelial Cells Specifically Activates Thrombin In Vitro

IL-6 has been shown to induce an increase in procoagulant activity of HUVECs in vitro. We have identified IL-6 to be one of the several proinflammatory cytokines that are upregulated by Tie-1 endodomain expression in endothelial cells. Therefore, we investigated whether expression of Tie-1 endodomain in HUVEC monolayer would activate thrombin in vitro.

Methods: HUVECs were grown to confluency in a 12-well plate. Cells were either uninfected, infected with control GFP adenovirus, or Tie-1 endodomain adenovirus. Next day, medium was changed to fresh full EGM2-MV. 72-hr post infection, cells were washed with EBM2 basal medium (without phenol red), and 0.5 ml of assay medium [EBM2 basal medium (without phenol red)/10% human plasma (Calbiochem 527420)/200 μM chromogenic thrombin substrate (Calbiochem 539518)] was added. Hirudin (Calbiochem 377853, 100 U/ml) was added to one set of cells infected with Tie-1 endodomain. The assay was quenched by the addition of aprotinin (Sigma A1154, 5.6 U/ml). Absorbance at 405 nm was measured using a spectrophotometer and used as an indicator of thrombin activity. The assays were done in triplicate. All measurements were normalized and represented as a percent increase relative to the value obtained from the GFP-infected HUVEC samples.

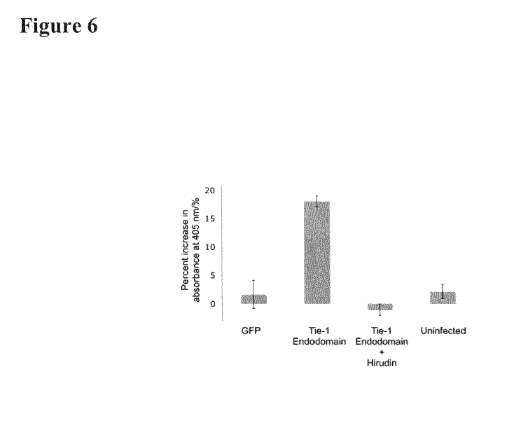

Results: We used a colorimetric assay to determine whether expression of Tie-1 endodomain could induce activation of thrombin in vitro. In these assays, normal human plasma (buffer exchanged into PBS) was added to the HUVEC monolayer together with an excess of a specific thrombin chromogenic substrate. Thrombin activation was detected by an increase in absorbance of the cleaved thrombin substrate (para-nitrophenol) at 405 nm. As shown in FIG. 6, adenoviral infection did not affect thrombin activation (compare GFP virus and uninfected). However, transient expression of Tie-1 endodomain in HUVECs induced a ˜20% increase in thrombin activation. This increase is specific to thrombin activity, because hirudin, a specific thrombin inhibitor, completely blocked the cleavage of the chromogenic thrombin substrate. Therefore, over-expression of Tie-1 endodomain in endothelial cells specifically activates thrombin in vitro. This finding is significant, because local activity of thrombin may induce endothelial activation, providing a proinflammatory environment favorable for atherosclerosis development.

### Example 6

### Thrombin Transactivates Multiple Receptor Tyrosine Kinases in Endothelial Cells In Vitro

Thrombin has been suggested to play a role in the development of vascular lesions. Intimal thickening in the early stage of atherosclerosis is, in part, a result of thrombus formation Tissue factor is highly expressed in atherosclerotic plaques. Since tissue factor is a critical initiating factor in the coagulation cascade, it is not unexpected to detect active thrombin in human atherosclerotic intima. However, no one has shown a connection between thrombin and Tie-1.

Our results showing expression of Tie-1 endodomain triggers thrombin activation is consistent with the notion that Tie-1 is proinflammatory and may modulate atherogenesis through the activity of thrombin. We hypothesized that thrombin achieves its diverse cellular responses in endothelial cells by transactivating multiple receptor tyrosine kinases.

Methods: Confluent HUVECs/RCC7 were pretreated with 1 mM sodium orthovanadate for 15 mins and stimulated with α-thrombin (Calbiochem) at 5 U/ml for 30 mins. Lysates were prepared according to the protocol included in the Phospho-RTK Array kit (R and D Systems). To validate the results obtained from this antibody array, thrombin-treated lysates were prepared in RIPA Buffer supplemented with 1× complete protease inhibitor (Roche), 2 mM sodium orthovanadate, 1 mM NaF, 2.5 mM β glycerol phosphate, 2.5 mM sodium pyrophosphate, and 1 mM EDTA. Tyrosine phosphorylated proteins were immunoprecipitated with 4G10 (Upstate) and captured with Protein A/G Plus (Santa Cruz). After SDS-PAGE, western blots were performed using the following antibodies: (a) anti phospho KDR (Y1054/Y1059) (Abcam 5473-50), (b) anti Tie-1 (Santa Cruz, C-18), (c) anti Tie-2 (Upstate, 05-584), (d) anti phospho EGFR (Y1068) (Cell Signaling, 2234), (e) anti EphA2 (Upstate, 05-480), (f) anti AXL (Cell Signaling, 4977), (g) anti phospho MERTK (Y749/753/754) (Abcam, 14921), (h) anti phospho RET (Y905) (Cell Signaling, 3221), (i) anti phospho c-MET (Y1234/1235) (Upstate, 07-211), and (j) anti phospho KDR (Y951) (Cell Signaling, 2471). To demonstrate that KDR is cleaved upon thrombin stimulation, HUVEC lysates prepared in RIPA buffer were immunoprecipitated with an anti KDR antibody (Santa Cruz, SC 6251) and immunoblotted with the same antibody.

Results: We used a phospho receptor tyrosine kinase antibody array to survey transactivation of receptor tyrosine kinases in HUVEC upon thrombin stimulation. In this assay, antibodies against the extracellular domain of 42 receptor tyrosine kinases were spotted on a membrane in duplicate. These antibodies captured the cognate receptors from the lysate. Activation status was evaluated using an anti phospho-tyrosine antibody conjugated to HRP. Therefore, if a receptor is expressed in HUVEC and is phosphorylated, two spots would appear at a specific location on the membrane upon chemiluminescence detection.

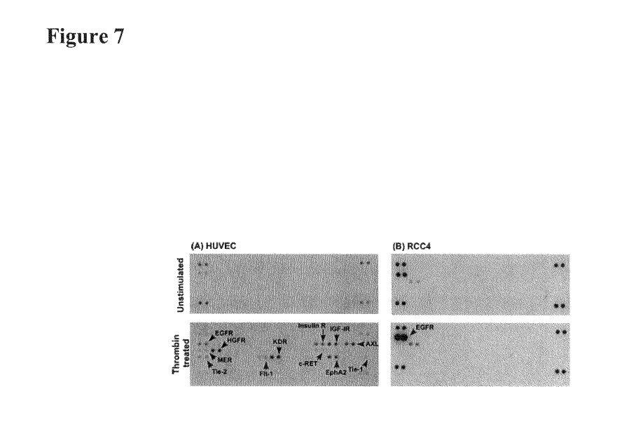

As shown in FIG. 7A, thrombin treatment induced significant phosphorylation of 12 receptor tyrosine kinases. They included EGFR, insulin receptor, IGF-IR, AXL, HGFR (c-met), Flt-1, KDR (VEGFR2), c-RET, MER, EphA2, Tie-1, and Tie-2. Receptor tyrosine kinases that were probed but were either not phosphorylated or not expressed in HUVECs included ErbB2, ErbB3, ErbB4, FGF R1, FGF R2α, FGF R3, FGF R4, Dtk, MSP R, PDGF Rα, PDGF Rβ, SCF R (c-kit), Flt-3, M-CSF R, ROR1, ROR2, TrkA, TrkB, TrkC, VEGF R3, MuSK, EphA1, EphA3, EphA4, EphA6, EpHA7, EphB1, EphB2, EphB4, and EphB6. When the same experiment was repeated with an epithelial cancer cell line (RCC4), only EGFR was significantly transactivated (FIG. 7B). Thus, the extensive cross talk between the thrombin receptor and receptor tyrosine kinases may be unique to endothelial cells.

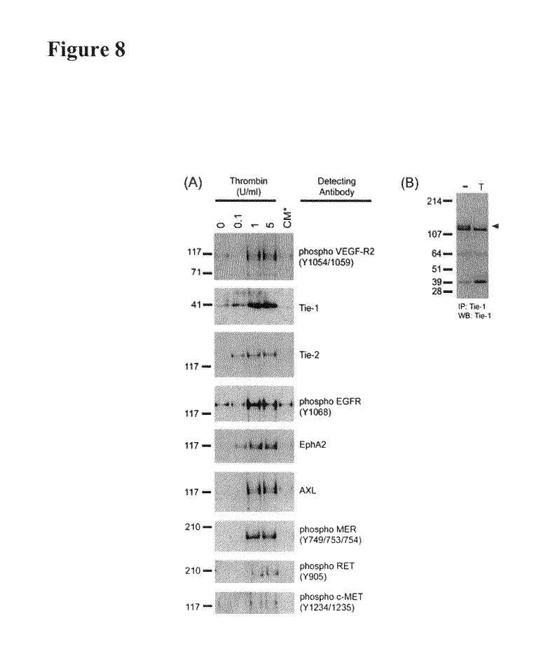

Next, we sought to validate the results from the antibody array. Because of the large number of candidates, we opted to immunoprecipitate tyrosine-phosphorylated cellular proteins with an anti phospho tyrosine antibody (4G10) and detect the identity of the phosphorylated proteins by western blotting with specific antibodies. As shown in FIGS. 8A-8B, we validated the results from the antibody array blot.

Consistent with published reports, thrombin stimulation of endothelial cells led to ectodomain shedding of Tie-1 (Yabkowitz et al., Blood 90: 706-715, (1997); Yabkowitz et al., Blood 93: 1969-1979, (1999)) (FIG. 2B). This may provide a ligand-independent activating mechanism of this orphan receptor. We showed here that the thus generated Tie-1 endodomain is tyrosine-phosphorylated (FIG. 8A). To our knowledge, this is the first study demonstrating tyrosine phosphorylation of Tie-1 endodomain. This finding is of particular interest. Tie-1 is overexpressed in atherosclerosis-prone sites. Our in vitro data suggest that expression of Tie-1 may activate thrombin locally, which in turn stimulates endothelial cells through PAR-1 and transactivates Tie-1. This scenario may set up an amplification loop of endothelial inflammation, triggering the onset of atherogenesis.

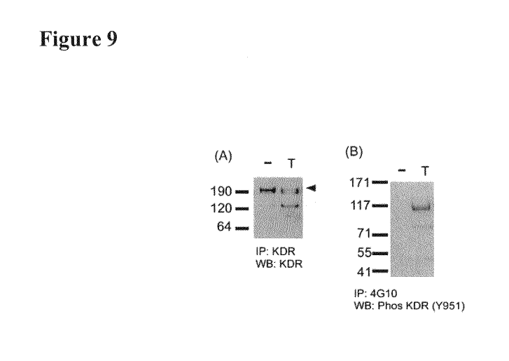

In the course of these experiments, we also noticed that the phosphorylated VEGFR2 band detected with an anti phospho VEGFR2 (Y1054/Y1059) was approximately 120 kDa in size, much smaller than the expected size of full-length VEGFR2 (about 180-230 kDa) (FIG. 8A). We hypothesized that thrombin stimulation resulted in shedding of part of the ectodomain of VEGFR2. To address this issue, we used an antibody directed against the C-terminus of VEGFR2 in immunoprecipitation experiments. As shown in FIG. 9A, the band corresponding to full length VEGFR2 (solid arrow) disappeared upon thrombin treatment, with the concomitant appearance of a VEGFR2 moiety of approximately 120 kDa in size (open arrow). To further verify the identity of the 120 kDa band was indeed a variant of VEGFR2, we performed western blotting using another phospho-specific VEGFR2 antibody (Y951). As shown in FIG. 9B, upon immunoprecipitation with 4G10 antibody, a band of 120 kDa was once again detected using this antibody, which is directed against a different epitope of activated VEGFR2, when HUVECs were treatment with thrombin. Taken together, our results strongly suggest that thrombin stimulation triggers ectodomain shedding of VEGFR2. To our knowledge, this observation has not been reported to date.

We then addressed whether the transactivation of receptor tyrosine kinases may be a result of release/secretion of growth factors induced by thrombin. We stimulated HUVECs with 5 U/ml thrombin for 30 mins. The supernatant was harvested and cell debris removed. The activity of thrombin was neutralized with excess hirudin (50 U/ml). We reasoned that any growth factors that were released as a result of thrombin stimulation should be present in this preparation. This supernatant was used immediately to stimulate a new batch of HUVECs. As shown in FIG. 8A (lane CM*), this supernatant failed to cause tyrosine phosphorylation of any of the receptor tyrosine kinases examined. Therefore, transactivation of these receptor tyrosine kinases upon thrombin stimulation was probably through an intracellular signaling pathway.

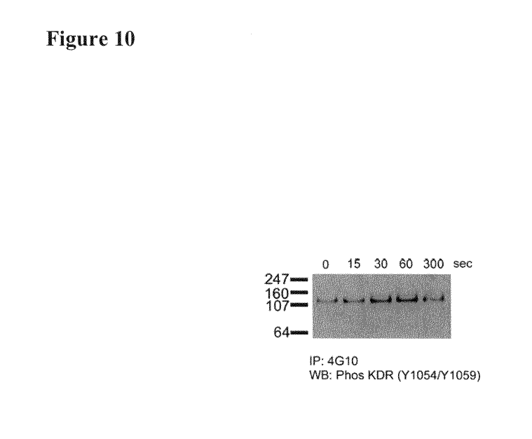

Next, we examined the time course of VEGFR2 activation by thrombin. No sodium orthovanadate pre-treatment was performed in this experiment. As seen in FIG. 10, thrombin rapidly activated VEGFR2. An increase in phosphorylation at Y1054/Y1059 was detected as early as 15 seconds. Therefore, transactivation of VEGFR2 appears to be an early downstream event of thrombin signaling.

Since VEGFR2 is activated by thrombin at an early time point, we decided to examine whether VEGFR2 activation is a pre-requisite for transactivation of other receptor tyrosine kinases by thrombin stimulation. We used a well-characterized RTK inhibitor SU5416. This small molecule inhibitor binds to the ATP binding pocket of the kinase domain of a subset of RTKs and prevents autophosphorylation of these receptors. It is most effective in blocking VEGFR-2 function in cells (Mendel et al., Clin Cancer Res 6: 4848-4858, (2000)). SU5416 has also been reported to inhibit the following receptors: PDGF receptor in cell based assays (20 times less effective), FLT3, c-kit/SCF R, and Flt-1. It does not inhibit EGFR and is a poor inhibitor of FGF R, IGF-IR, and c-Met. Since neither PDGFR nor c-kit/SCF R was activated by thrombin, as evidenced from our antibody array experiment (FIG. 1A), the use of SU5416 in our experiments would address the role of VEGFR1/2 in thrombin-induced endothelial functions.

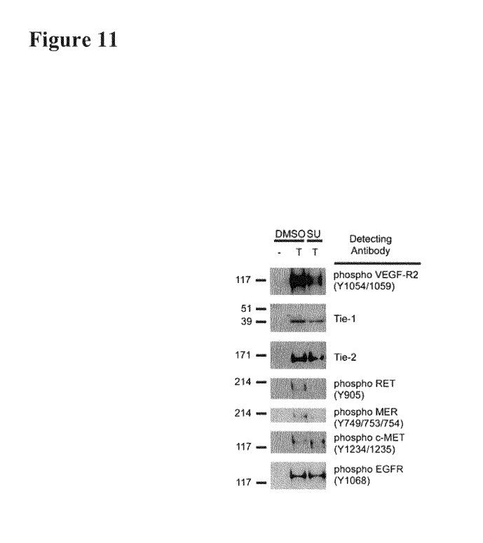

As expected, pre-incubation of confluent HUVECs with SU5416 significantly attenuated phosphorylation of VEGFR2 induced by thrombin (FIG. 11). Interestingly, phosphorylation of several of the RTKs induced by thrombin was also blocked by SU5416 pre-treatment. For example, activation of RET and MER by thrombin stimulation was completely blocked in the presence of SU5416. Thrombin-induced phosphorylation of Tie-1 endodomain and Tie-2 was partially blocked by SU5416. In contrast, neither the activation of c-MET nor EGFR by thrombin was affected by SU5416. Taken together, these results suggest that VEGFR may serve as a downstream effector of thrombin stimulation to expand the intracellular signaling network by transactivating other receptor tyrosine kinases.

### Example 7

### Thrombin-Induced Adherens Complex Disruption and Endothelial Gap Formation In Vitro Requires VEGFR Activity

Since VEGFR activation by thrombin was an early event and appears to play a critical role in the transactivation of other RTKs, we concentrated on understanding the precise role of VEGFR in thrombin-induced endothelial inflammation. One of the earliest responses of endothelial monolayer to thrombin stimulation is gap formation. Therefore, we asked whether inhibition of VEGFR activation would affect thrombin-induced endothelial gap formation.

### Methods

Permeability assay: 50,000 HUVECs were seeded in a Transwell insert (Corning 3496) in 100 μl of full EGM2-MV medium. EGM2-MV medium (600 μl) was added to the lower chamber. An endothelial monolayer was allowed to establish overnight. The next day, HUVECs were pre-treated with 10 μM SU5416 or DMSO for 1 hour, followed by the addition of 200 μg/ml FITC-BSA with or without 5 U/ml thrombin to the Transwell inserts. The inserts were then immediately transferred to a new well containing 500 μl PBS (with Mg²⁺/Ca²⁺; GIBCO 14040-141). After 10 mins of incubation, the Transwells were removed, and fluorescence of the PBS in the lower chamber was measured using a CytoFluor Multiwell Plate Reader (Series 4000) with the following settings: excitation λ 485/20 mm, emission λ 530/25.
Immunostaining: LabTek slide chambers (177429) were precoated with 50 μg/ml rat tail collagen type-1 (BD Biosciences, 354236) at 37° C. for 1 hour according to the manufacturer's protocol. Each slide chamber was seeded with 200,000 HUVECs in one ml of EGM2-MV medium. An endothelial monolayer was allowed to established overnight. The next day, cells were pre-treated with 10 μM SU5416 or DMSO for 2 hours, after which thrombin (or PBS) was added to 5 U/ml. After 15 mins of stimulation, cells were fixed with 4% paraformaldehyde in PBS for 15 mins and permeabilized with 0.5% Triton X-100 in PBS for 5 mins. After blocking in 0.5% FBS/PBS at room temperature for 1 hour, a mouse VE-Cadherin antibody (BD Biosciences 610251; 1:100 in 0.5% FBS/PBS) was applied for one hour, followed by three washes with 0.5% FBS/PBS. Next, the samples were incubated in 0.5% FBS/PBS containing an ALEXA-488-conjugated anti-mouse antibody (Probes, A11029) and ALEXA-546-conjugated phalloidin (Probes, A22283) for one hour. After 3 washes with 0.5% FBS/PBS, the samples were mounted in a drop of ProLong Gold antifade reagent with DAPI (Probes, P36931). Images were recorded using a fluorescence microscope (Nikon Corporation, Tokyo, Japan) and coupled to a Spot RT camera (Diagnostic Instruments Inc, Sterling Heights, Mich.).
Western blotting: To examine the phosphorylation status of VE-Cadherin and myosin light chain

(MLC) upon thrombin stimulation, a procedure derived from a published protocol was used (Ukropec et al., J. Biol. Chem. 275: 5983-5986, (2000)). Confluent HUVECs were pre-treated with either 10 μM SU5416 or DMSO for 2 hours and stimulated with thrombin (1 U/ml) for 5 mins. Cells were then washed with PBS containing Mg²⁺/Ca²⁺ (GIBCO 14040-141) supplemented with 1 mM Na₃VO₄ and 0.2 mM H₂O₂ at room temperature for 5 mins. Cells were then lysed in 1 ml of ice-cold lysis buffer (1% TritonX100/20 mM HEPES pH 7.5/50 mM NaC1/3 mM Na₄P₂O₇/50 mM NaF/2.5 mM glycerol 13 phosphate/2 mM Na₃VO₄/2 mM H₂O₂/1× Protease Complete. After rocking at 4° C. for 30 mins, cell debris was removed by centrifugation. A portion of the clarified lysate was used for ascertaining MLC phosphorylation status by western blotting using a phospho-specific MLC antibody (Cell Signaling, 3671, 1:1000). The membrane was stripped and reblotted for GAPDH (Chemicon, MAB 374, 1:5000) for loading. The rest of the clarified lysate was immunoprecipitated with a goat polyclonal anti VE-cadherin antibody (Santa Cruz 6458). After SDS-PAGE, phosphorylation of VE-cadherin was detected by western blotting with the antibody 4G10. The membrane was then stripped and reblotted with a mouse monoclonal anti VE-cadherin antibody (BD Transduction 610251, 1:500) for loading. Lysates used in p120 phosphorylation assay were prepared as described above. After immunoprecipitation with 4G10, western blotting was performed using a goat polyclonal anti p120 antibody (Santa Cruz, SC-1730, 1:1000).

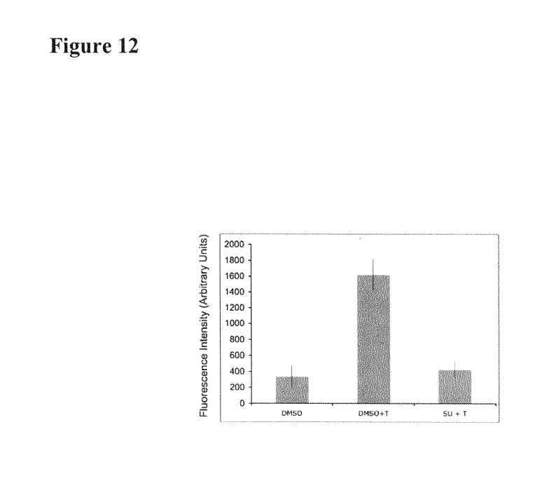

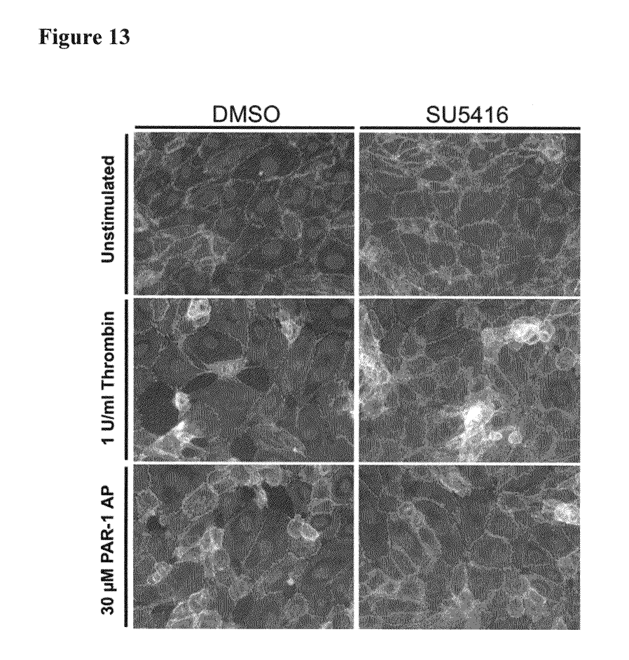

Results: As expected, thrombin induced a significant increase in endothelial permeability (FIG. 12). This effect was completely blocked in the presence of SU5416, strongly suggesting that VEGFR plays a critical role in thrombin-induced endothelial permeability in vitro. To further investigate the contribution of VEGFR to thrombin's action, we performed fluorescence immunostaining of HUVEC monolayer before and after thrombin stimulation. As shown in FIG. 13, VE-cadherin (green) tightly localized at the endothelial junctions prior to thrombin treatment. In addition, only very low amounts of actin stress fiber (red) were detected in this basal condition. Neither DMSO alone nor SU5416 had observable effects on VE-cadherin organization and stress fiber formation. Addition of thrombin (1 U/ml) significantly disrupted VE-cadherin pericellular localization and induced the formation of actin stress fiber. Consequently, large inter-cellular gaps were easily observable. Similar results were obtained with a PAR-1 agonistic peptide (PAR-1 AP, 30 μM). While SU5416 did not block stress fiber formation induced by thrombin, it completely blocked VE-cadherin disruption induced by thrombin or PAR-1 AP, and no intercellular gaps formed. These data are very consistent with our observation that SU5416 completely blocked thrombin induced endothelial permeability (FIG. 12). The data presented here also suggest that VEGFR activation is downstream of PAR-1 and is required for thrombin-induced endothelial permeability (likely via a VE-cadherin dependent pathway).

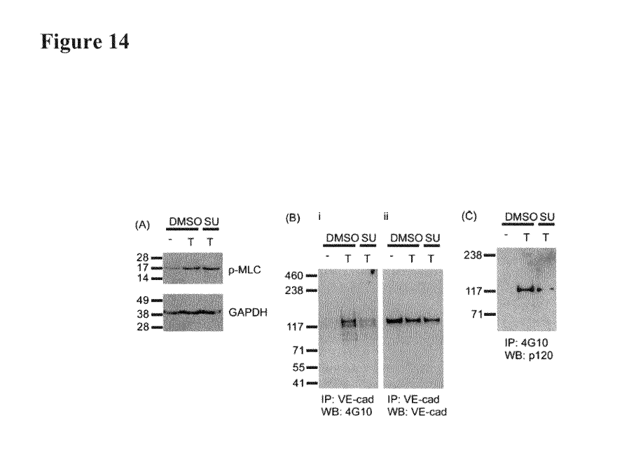

Next, we examined phosphorylation of myosin light chain (MLC) and VE-cadherin, two major intracellular signaling pathways that have been shown to be important in endothelial gap formation upon thrombin stimulation. As shown in FIG. 14A, thrombin, as expected, induced phosphorylation of MLC at serine 19. This signaling pathway apparently is independent of VEGFR, because pretreatment of HUVECs with SU5416 did not affect serine phosphorylation of MLC induced by thrombin. Since MLC signaling plays a critical role in stress fiber formation in endothelial cells, this result is in agreement with our immunostaining results indicating that VEGFR inhibition did not reduce stress fiber formation induced by thrombin or PAR-1 activating peptide (FIG. 13).

Since VEGFR inhibition blocked thrombin-induced VE-cadherin redistribution (FIG. 13), we examined changes of VE-cadherin and p120 catenin at the molecular level. Thrombin-induced tyrosine phosphorylation of VE-cadherin (FIG. 14B) and p120 (FIG. 14C) was significantly blocked by SU5416. These results are in excellent agreement with the notion that tyrosine phosphorylation of proteins comprised of the adherens junction governs endothelial permeability. Regulation of adherens junctions by thrombin is thought to involve protein-tyrosine phosphatase SHP-2 (Ukropec et al., J. Biol. Chem. 275: 5983-5986, (2000)). Upon stimulation, SHP-2 dissociates from VE-cadherin, followed by tyrosine phosphorylation of VE-cadherin, p120, β catenin, and γ catenin (Ukropec et al., J. Biol. Chem. 275: 5983-5986, (2000)). These molecular changes lead to endothelial barrier breakdown. Indeed, tyrosine phosphorylation of adherens junction components, including VE-cadherin, β-catenin, and p120, decreases dramatically as endothelial cells grow from subconfluent to confluent state (Lampugnani et al., J. Cell Sci. 110 (Pt 17): 2065-2077, (1997)). Furthermore, phosphorylation of tyrosine residues 658 and 731 of VE-cadherin prevents its binding to p120 and β catenin and reduces cell-barrier function (Potter J. Biol. Chem. 280: 31906-31912, (2005)). Collectively, our data provide compelling evidence that VEGFR may serve as a mediator to relay signals from PAR-1 to VE-cadherin, resulting in dismantling of endothelial adherens junctions.

Examples 1-7, described above, demonstrate that expression of the endodomain of Tie-1 in endothelial cells elicits a proinflammatory response as judged by three parameters:
1) Induction of expression of specific proinflammatory cytokines (IP-10, IL-6, and G-CSF);
2) Upregulation of adhesion molecules (VCAM-1 and ICAM-1); and
3) Activation of thrombin.
   Consistent with the enhanced expression of adhesion molecules, we have shown that binding of monocytes to HUVECs expressing Tie-1 endodomain is elevated, one of the earliest detectable cellular responses in the formation of lesions of atherosclerosis. Our results demonstrate that expression of the endodomain of Tie-1 in endothelial cells provides a favorable environment for atherosclerosis to develop by presenting both chemotactic and retention signals for leukocytes to migrate and attach to the endothelium. In addition, Tie-1 endodomain also promotes migration of smooth muscle cells, another key cellular response observed in atherosclerotic lesions. Furthermore, Tie-1 endodomain expression triggers activation of thrombin, which may exert its effect locally on endothelial cells and may be an important molecular step in the development of atherosclerosis. Little is known about the signaling pathway of thrombin-mediated endothelial cell activation and the examples above provide the identification of some signaling molecules that are involved in this signaling pathway. We found that multiple receptor tyrosine kinases are transactivated in endothelial cells upon thrombin stimulation. One of them is VEGFR2, which appears to be critical in mediating thrombin-induced endothelial gap formation through regulating VE-cadherin stability. The discovery of transactivation of receptor tyrosine kinases provides a unique opportunity to inhibit thrombin-mediated endothelial inflammation responses using small molecule receptor tyrosine kinase inhibitors without interfering with thrombin ability to promote fibrin clot formation.

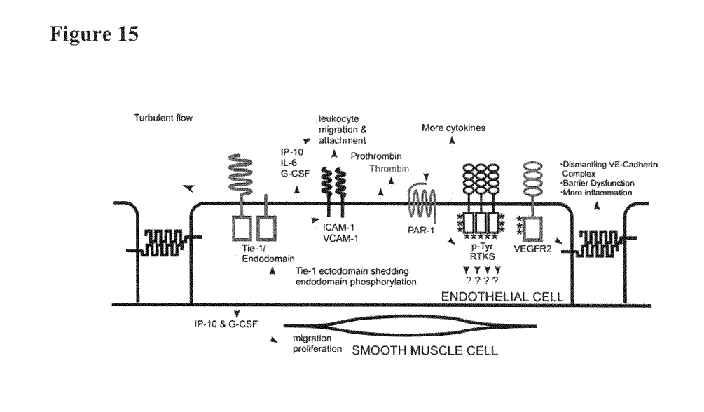

FIG. 15 provides a proposed working model on how Tie-1 affects endothelial inflammation and how it may be a key precipitating molecular factor that triggers the onset of atherosclerosis based on in vitro data described above. At arterial branch points, endothelial cells experience unusually high turbulent flow. This hemodynamic condition upregulates Tie-1 expression and its activation, possibly through ectodomain shedding. Proinflammatory cytokines, such as IP-10, IL-6, and G-CSF, and adhesion molecules ICAM-1 and VCAM-1 are subsequently induced. These responses lead to recruitment and attachment of leukocytes from blood and proliferation and migration of smooth muscle cells in the intimal layer. Additionally, prothrombin to thrombin conversion is enhanced. Locally generated thrombin may then activate PAR-1, which is abundantly expressed in endothelial cells. Activation of endothelial cells by thrombin not only induces upregulation of more inflammatory cytokines but also transactivates multiple receptor tyrosine kinases. Through the activity of VEGFR2, thrombin induces the dismantling of VE-cadherin complexes. Exposure of basal membrane components such as collagen or tissue factor due to endothelial gap formation further amplifies the inflammatory response. Since Tie-1 is one of the receptor tyrosine kinases that is transactivated by thrombin through PAR-1, an amplification loop may set up, providing an environment for atherosclerosis to develop. In the Examples described below, we test the functional role of Tie-1, PAR-1, and VEGFR2 (FIG. 15, red) expression/activity in mice plays an essential role in the pathobiology of atherosclerosis.

### Example 8

### Assays to Examine the Role of IP-10, IL-6, G-CSF, ICAM-1, and VCAM-1 in Endothelial Inflammation Induced by Tie-1 Endodomain

The role of IP-10, IL-6, G-CSF, ICAM-1, and VCAM-1 in endothelial inflammation induced by Tie-1 endodomain are assayed using antibody blockade experiments. Using the methods described herein, we can identify intracellular signaling pathways that are responsible for Tie-1 endodomain-induced upregulation of these proinflammatory markers.

We have shown that adhesion of U937 cells to HUVECs is enhanced by the expression of Tie-1 endodomain (FIGS. 3A-3C). This is likely due to the upregulation of ICAM-1 and VCAM-1 on the endothelial cell surface (FIGS. 2F and G). Likewise, the stimulation of smooth muscle cell migration by the conditioned medium produced by HUVE cells expressing the endodomain is, in part, due to the increased level of IP-10 or G-CSF (FIGS. 2A-D and I). In addition, Tie-1 endodomain promotes activation of thrombin (FIG. 6). The mechanism for this coagulation response may be a result of IL-6 upregulation (FIG. 2H), since IL-6 stimulates tissue factor expression in HUVECs in vitro, resulting in an increase procoagulant activity. We hypothesize that IP-10, IL-6, G-CSF, ICAM-1, and VCAM-1 mediate the cellular responses observed when Tie-1 endodomain is overexpressed. Antibody blockade experiments to assess the role of each molecule in these functional assays.

Methods: The following antibodies can be purchased from R and D Systems: 1) Monoclonal anti human CXCL-10/IP-10 antibody (MAB266); 2) monoclonal anti-human ICAM-1 antibody (BBA3); 3) polyclonal anti-VCAM-1 antibody (AF809); 4) monoclonal anti-human IL-6 antibody (MAB 227); 5) monoclonal anti-human G-CSF antibody (MAB214). These antibodies have been tested by the manufacturer to be functionally neutralizing.

U937 adhesion assays: First, the blocking antibodies are characterized using HUVECs that have been pre-treated with TNF-α (10 ng/ml) for 4 hours. Increase in both ICAM-1 and VCAM-1 expression has been reported with this treatment and will be verified and quantified by western blotting. Attachment of U937 cells to the activated HUVECs is performed in the absence or presence of increasing amounts of the blocking antibody. At the beginning, only one antibody is used to obtain a dose that achieves maximum inhibition of attachment. Then, both antibodies (anti VCAM-1 and anti ICAM-1) will be applied at maximum effective doses to block U935 cell attachment. Next, the importance of ICAM-1 and VCAM-1 in Tie-1 endodomain-induced U937 adhesion is assessed using HUVE cells will be transduced with either GFP or Tie-1 endodomain adenovirus as described in Example 1. The magnitude of ICAM-1 and VCAM-1 upregulation is determined by real-time PCR and compared to that in TNFα-treated HUVECs. This comparison will provide a guideline for the doses of the blocking antibodies to use. HUVE cells are pretreated with the antibody against either ICAM-1 or VCAM-1 (or in combination) at various concentrations prior to the addition of U937 cells. The optimal length of pre-incubation period is determined empirically. A control antibody that matches the Ig class can be used in each experiment as controls.
Smooth muscle cell migration assay: The optimal dose of blocking antibody and pre-incubation time is determined using purified, recombinant human IP-10 and G-CSF (R and D) at a concentration similar to that in the conditioned medium (as determined by ELISA, see FIG. 2B for example). The conditioned medium is pre-incubated with anti-IP-10 antibody under those conditions prior to use as a stimulant for migration. The contribution of G-CSF to smooth muscle cell migration can also be tested using a similar procedure. Finally, a combination of the two blocking antibodies at their respective maximum effective doses is tested in blocking smooth muscle cell migration induced by the HUVEC conditioned medium.
In vitro thrombin activation assay: We can determine by antibody blockade experiments whether activation of thrombin induced by the expression of Tie-1 endodomain is IL-6 dependent. The experiments are performed as follows. IL-6 blocking monoclonal antibody is added to HUVECs 24 hr post transduction with either GFP or Tie-1 endodomain adenovirus. Activity of thrombin is determined using the chromogenic thrombin substrate 72 hr post transduction as described above. Next, we can examine whether the elevated thrombin activity induced by Tie-1 endodomain expression is due to upregulation of tissue factor in HUVECs. This is achieved in two steps. First, we determine whether tissue factor is upregulated upon Tie-1 endodomain expression by real-time PCR and western blot analysis. Next, we employ a neutralizing anti-tissue factor antibody in our in vitro thrombin activation assay to ascertain whether tissue factor contributes to the increase in thrombin activity observed when Tie-1 endodomain is overexpressed in HUVECs.

In addition, siRNA knockdown technology can also be used. Small siRNAs specific to ICAM-1, VCAM-1, IP-10, IL-6, G-CSF, and tissue factor are available from Ambion. Three sequences per target will be tested (IP-10: ID#10111, 10020, 144783; ICAM-1: ID#144512, 105997, 105995; VCAM-1: ID#138776-8; IL-6: ID#199821-3; tissue factor: ID#10909, 10904, 146260; G-CSF: ID#8910, 9005, 9094). siRNA duplexes are transfected into HUVECs using SilentFect (BioRad), because we have achieved high gene-knockdown and low toxicity in HUVECs in other experiments. Efficiency of knockdown is determined by real-time PCR.

A different antibody array can also be used to screen for upregulation of other cytokines by Tie-1 endodomain. Two examples are the RayBio Human Inflammation Antibody Array and the Human Atherosclerosis Antibody Array. Alternatively, a microarray analysis can be performed using cDNA prepared from endothelial cells expressing Tie-1 endodomain.

### Example 9

### Assays for Identification of the Mechanism by which the Expression of Tie-1 Endodomain Leads to Thrombin Activation

Expression of Tie-1 endodomain in HUVECs induces activation of thrombin in vitro (FIG. 6). The assays described below can be used to identify the mechanism by which Tie-1 endodomain exerts this effect. Since IL-6 expression is upregulated by Tie-1 endodomain expression and IL-6 has been shown to increase HUVEC's procoagulant activity in vitro through tissue factor induction, we use IL-6 as an example for the methods below.

Methods/Data Interpretation: First, the amount of IL-6 secreted in response to Tie-1 endodomain expression is determined by ELISA (R&D, D6050). Next, recombinant, purified human IL-6 (R&D, 206-IL) at this dose is added to HUVEC and the procoagulant activity is determined as described above. Antibody blockade experiments re performed using a neutralizing anti human IL-6 polyclonal antibody from R and D Systems (AB206-NA). The dose of the antibody needed to achieve maximum inhibition of procoagulant activity induced by recombinant IL-6 is noted. Next, the neutralizing antibody is applied to HUVECs expressing the Tie-1 endodomain. The doses used will bracket the maximum inhibitory dose established earlier. A decrease in procoagulant activity in the presence of the neutralizing antibody suggests that IL-6 plays a role in thrombin activation by Tie-1 endodomain expression.

Assays to determine tissue factor is involved in thrombin activation include the use of RNA inference. Three siRNA specific to human tissue factor (NM_{—}001993) can be purchased from Ambion (#146260, 10904, and 10809). Delivery of siRNA oligos into endothelial cells is achieved using SilentFect (Bio-Rad) (Parikh et al., PLoS Med 3: e46, (2006)). The specificity of these siRNAs is tested by stimulating the endothelial cells with TNFα (10 ng/ml), which is a potent inducer of tissue factor. Efficiency of expression knockdown is determined by real-time PCR. Once the gene-knockdown ability of the siRNAs has been established, they are used to determine whether tissue factor is involved in thrombin activation by Tie-1 endodomain as follows. After transfection with the siRNAs, expression of Tie-1 endodomain is induced by adenoviral infection. 48-hr post infection, thrombin activity is determined using the chromogenic assay described above. A decrease in thrombin activity will indicate that tissue factor mediates Tie-1-endodomain-induced thrombin activation in endothelial cells.

To further investigate the role of p38 activation in endothelial inflammation induced by Tie-1 endodomain, a pyridinyl imidazole type p38 specific inhibitor SB-203580 (Calbiochem) is used. HUVE cells are infected with either the GFP- or Tie-1 endodomain adenovirus at MOI˜10:1. Five hours post infection, growth medium is changed, and SB-203580 is added (0 to 10 μM). Mock treatments are performed in parallel by addition of equal amount of vehicle (DMSO). After 48 hours of incubation, the level of proinflammatory cytokines, IP-10, for example, in the growth medium is determined by ELISA. Effects at the RNA level can also be determined by real-time PCR. Additionally, monocyte adhesion and smooth muscle cell migration assays are performed under similar conditions.

The role of MKK3/6 and MKK4 in the activation of p38 caused by Tie-1 endodomain expression can be probed by Western blot analysis using antibodies specific to the phosphorylated MKK3/6 or MKK4 (Cell Signaling). MKK4 activation will be used as an example to illustrate the strategy. HUVE cells are infected with either GFP or Tie-1 endodomain adenovirus. A time course of activation of both p38 and MKK4 is determined using phospho-specific antibodies.

The role of NFκB in Tie-mediated inflammation can be determined using a NFκB ELISA kit (Panomics). IκB phosphorylation inhibitor BAY 11-7082 (Calbiochem) can be used to test the role of NFκB in Tie-1 endodomain-induced IP-10 upregulation. HUVE cells are infected with Tie-1 endodomain adenovirus for 5 hours, at which point the cells are incubated with fresh medium containing various amounts of BAY 11-7082 (0 to 1 μM). 48 hrs post infection, the protein level of IP-10 in the medium is determined by ELISA. Since it is a 48-hr assay, fresh BAY 11-7082 may be added within the assay period. A decrease in IP-10 expression in the presence of BAY 11-7082 will indicate that Tie-1 endodomain upregulates IP-10 via NFκB.

### Example 10

### Use of a Transgenic Mouse Line that Conditionally Suppresses Tie-1 Expression Via RNA Interference to Assay Tie-1 Mediated Inflammatory Pathways

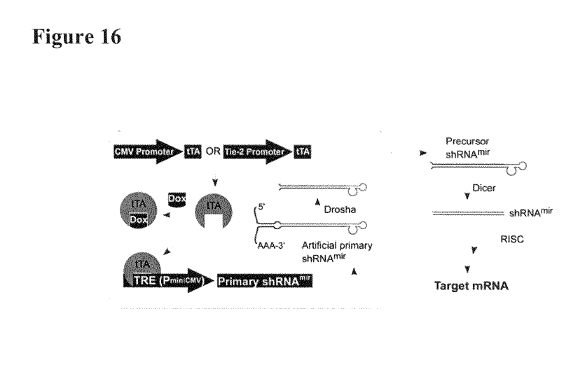

For these experiments, the Tie-1/Tie-1 endodomain can be overexpressed in the endothelium of a transgenic mouse line for a gain of function model to test whether expression of Tie-1/Tie-1 endodomain is sufficient to initiate atherogenesis in mice and to assay candidate Tie-1 inhibitor compounds. In addition, a model which conditionally suppresses the expression of Tie-1 can be used to test whether Tie-1 expression is necessary for atherosclerosis to develop. For the suppression model, a temporal regulation of expression knockdown can be achieved using the Tet-Off system to express a Tie-1-specific microRNA upon tetracycline withdrawal. The Tet-Off method is used because it has been shown to exhibit higher degree of gene knockdown compared to the Tet-On method using this shRNA-miR30 system. To achieve gene knockdown, a microRNA strategy was chosen over the traditional shRNA method because of its higher gene targeting efficiency even at single-copy integration level (Dickins et al., Nat Genet 37: 1289-1295, (2005); Stegmeier et al., Proc. Natl. Acad. Sci. 102: 13212-13217, (2005)). The microRNA will be embedded in and transcribed as an artificial primary shRNAmir of miR30 in the absence of doxycycline (FIG. 16). Specific RNA processing will generate the targeting microRNA.

The plasmid pTet-Off (Clontech) can be used for creating this line. The BsrGI/HindIII fragment of this plasmid contains the CMV promoter, the coding sequence of tTA, and a polyadenylation signal. This fragment will be used for microinjection (see below)

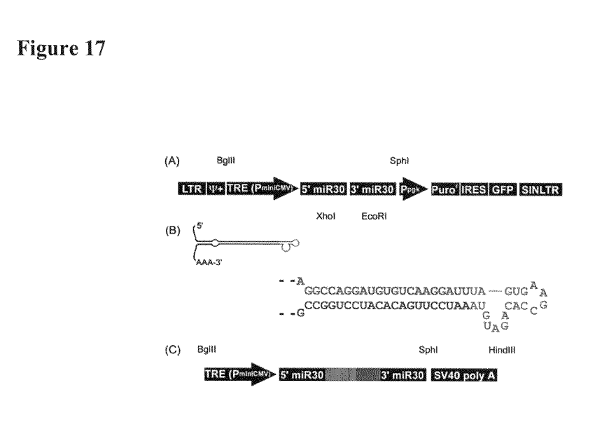

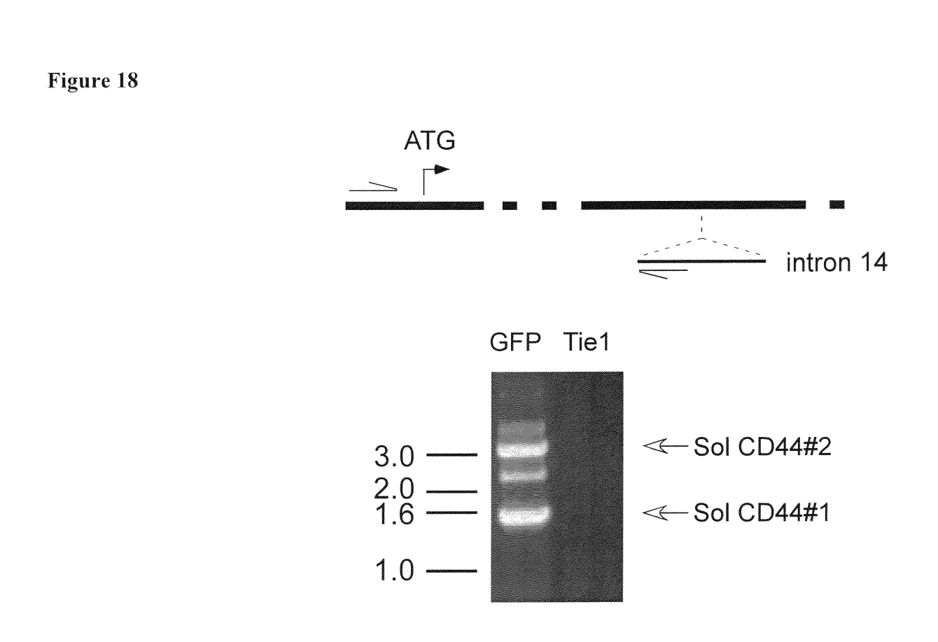

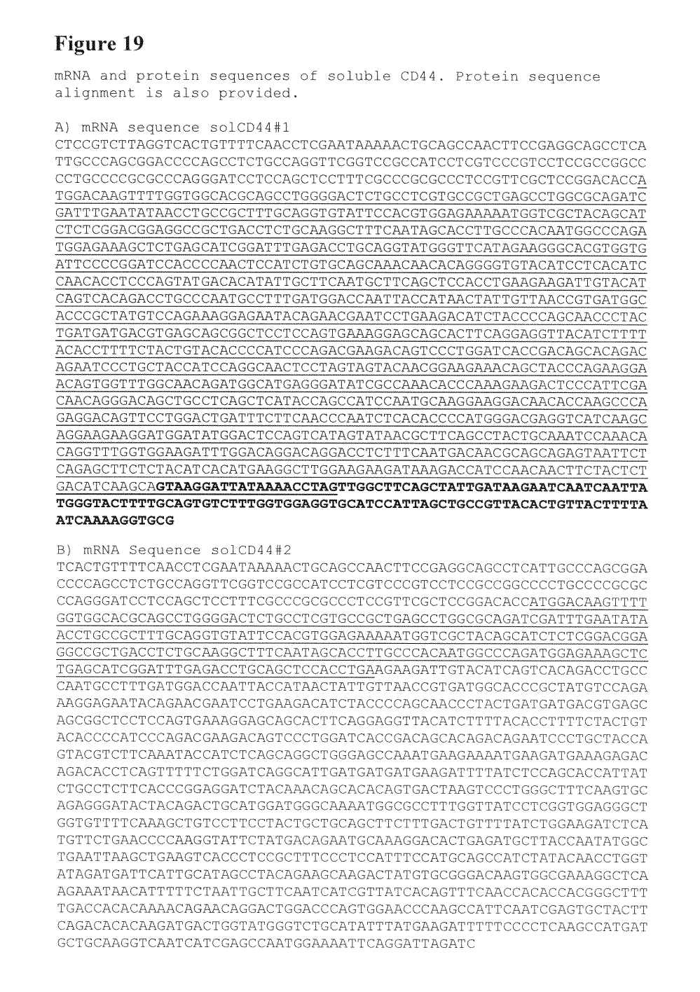

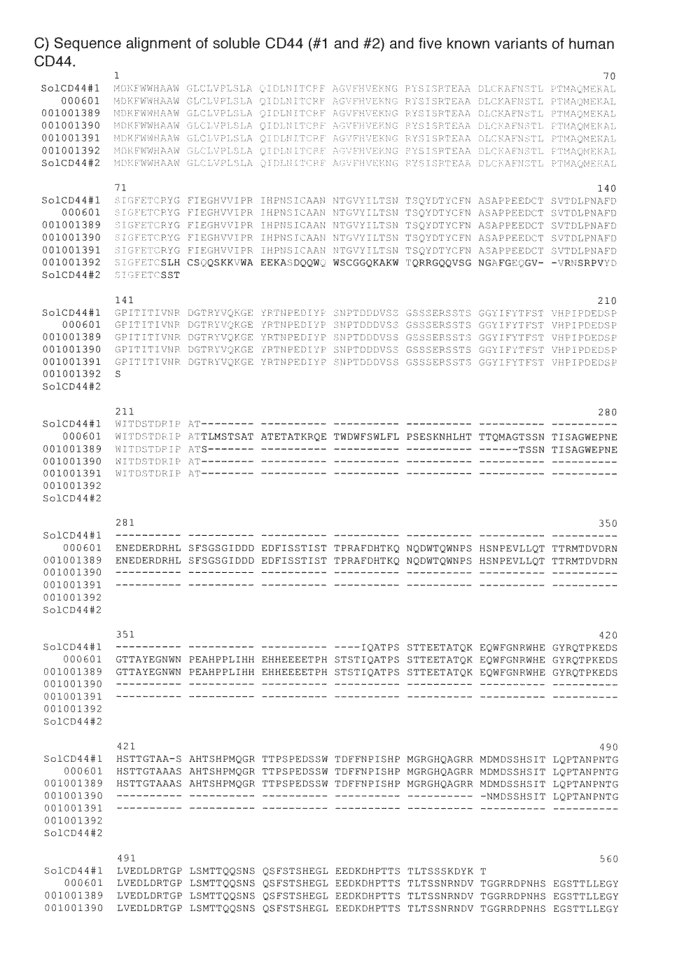

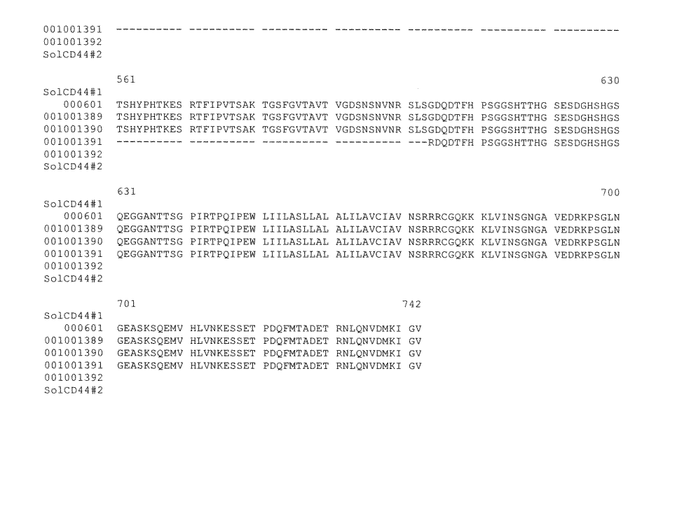

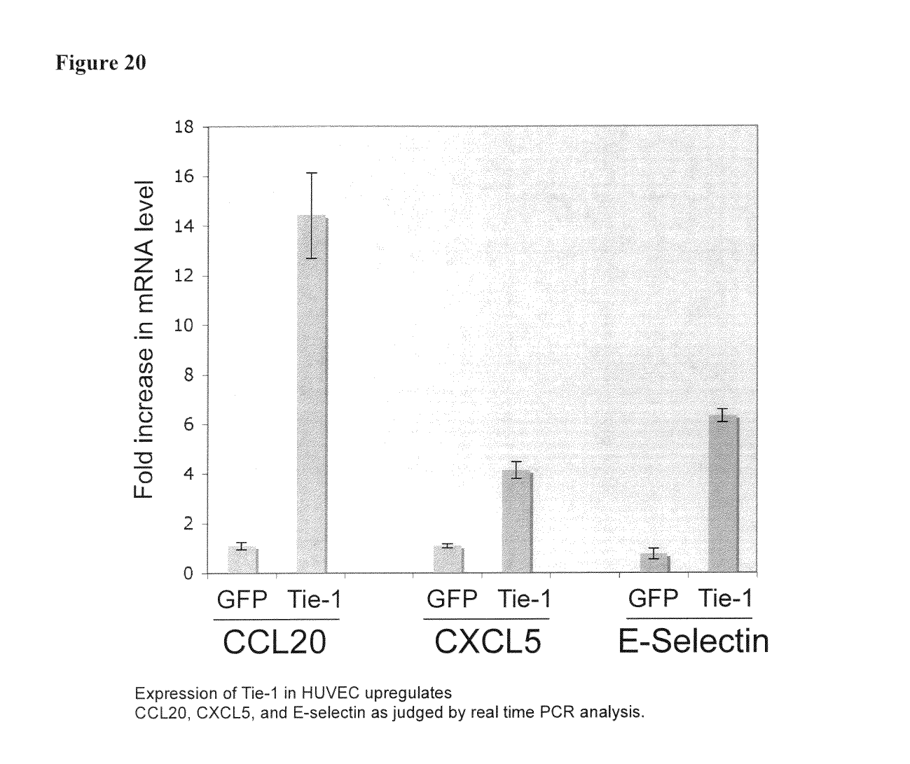

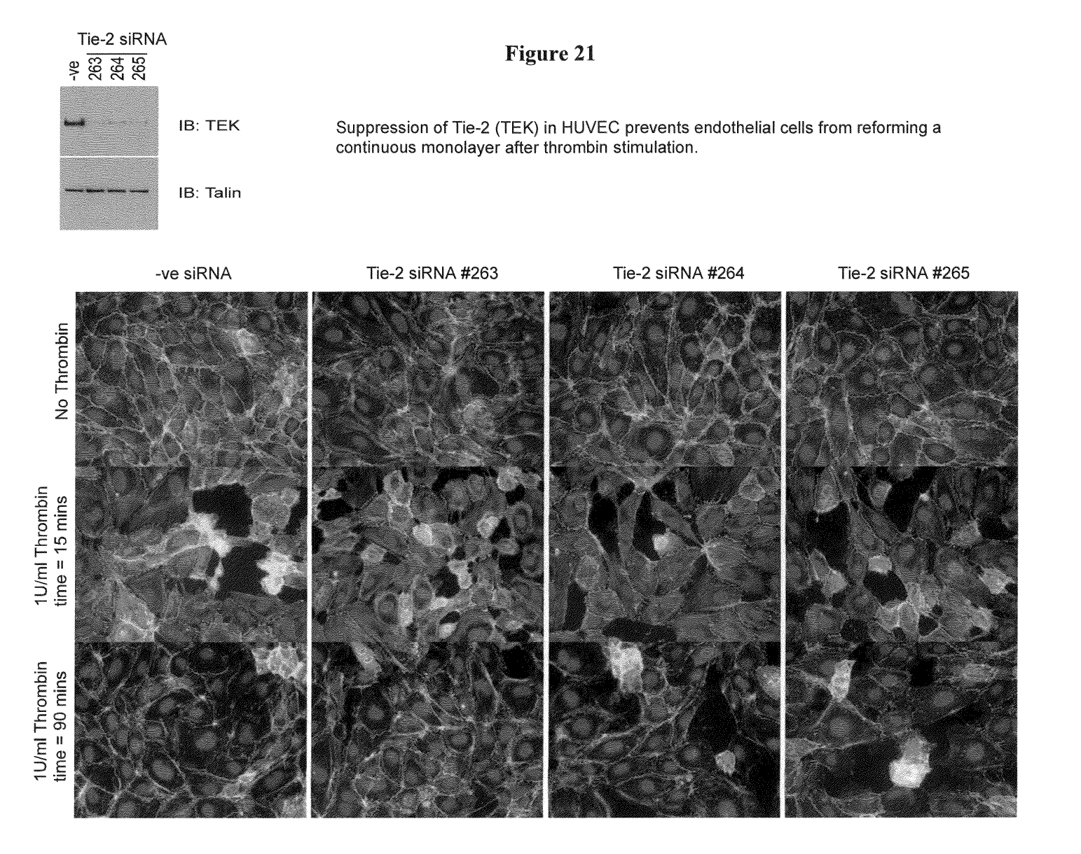

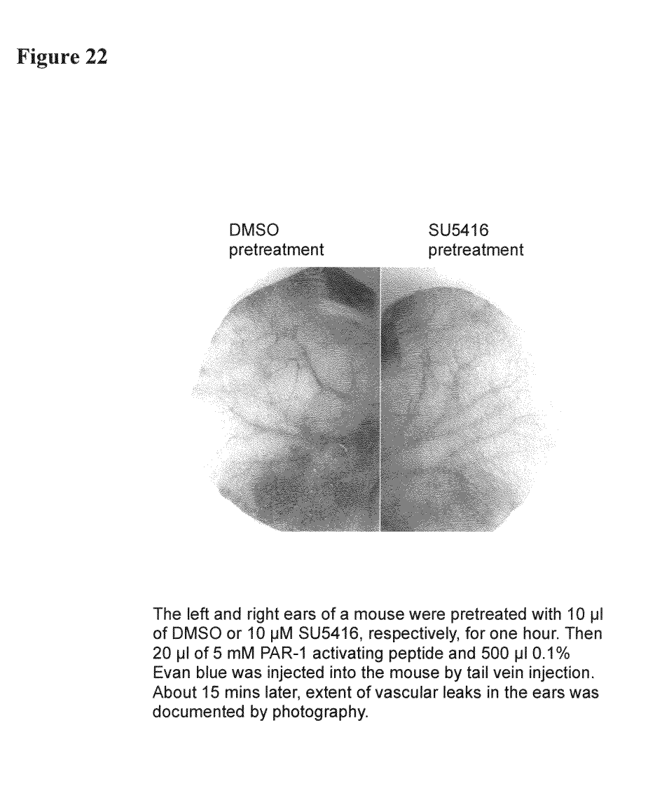

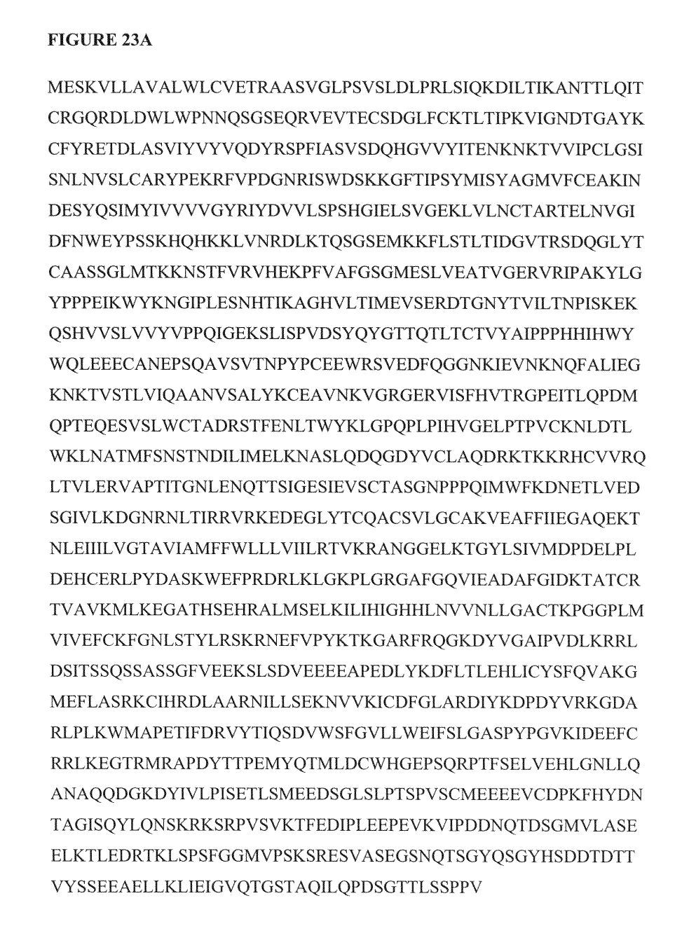

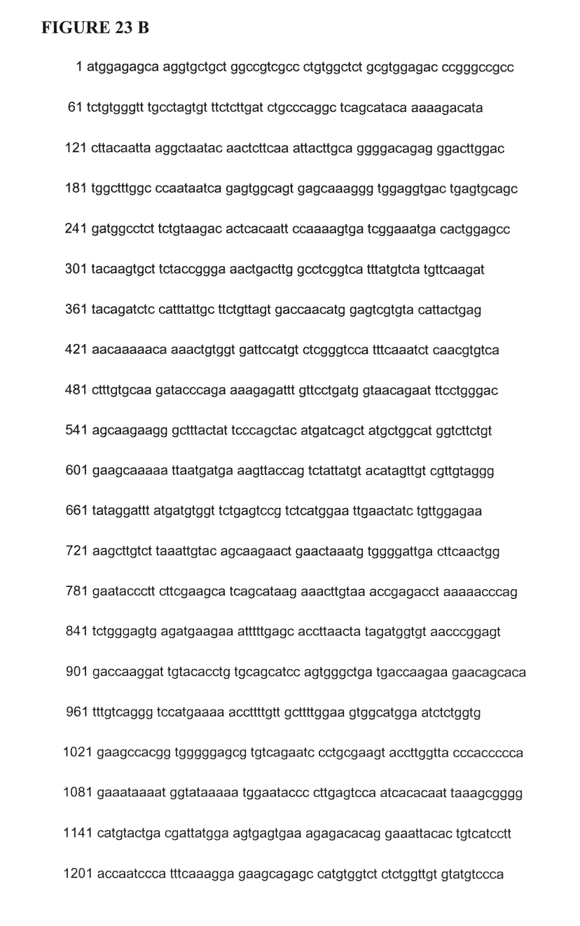

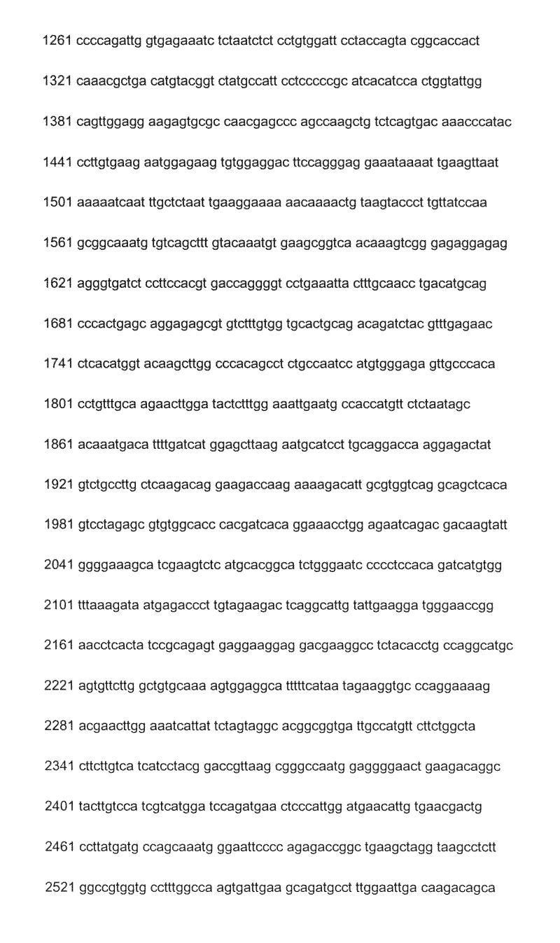

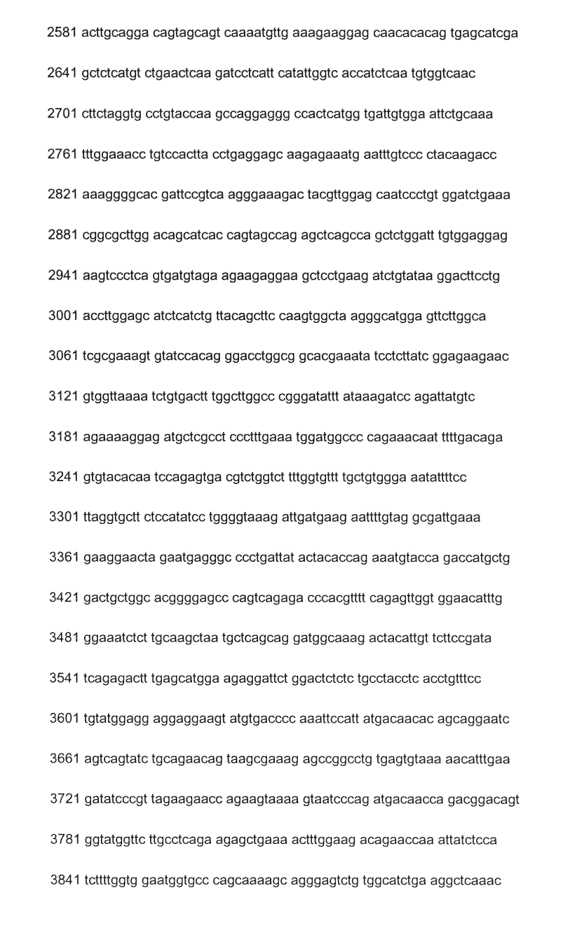

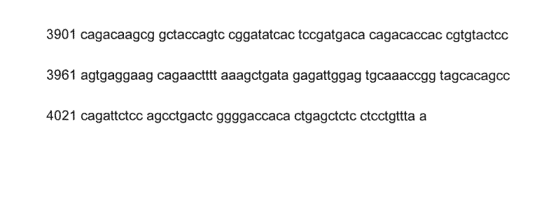

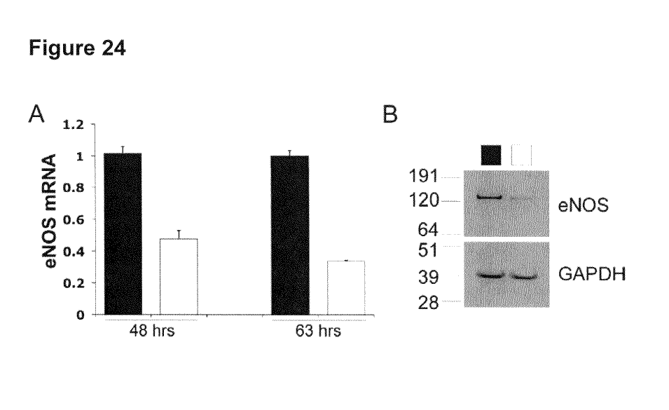

A retroviral vector named SIN-TREmiR30-PIG (TMP) (OpenBiosystem) issued to express Tie-1 microRNA (FIG. 17A). Three microRNAs targeting mouse Tie-1 will be prepared. The sense (FIG. 17B, red) and the antisense (FIG. 17B, blue) sequences are generated at RNAi Central (http://katandin.cshl.org:9331/homepage/siRNA/RNAi.cgi?type=shRNA):

Construct #1:
(SEQ ID NO: 15)
5′-AGGCCAGGATGTGTCAAGGATT-3′
and
(SEQ ID NO: 16)
5′-AATCCTTGACACATCCTGGCCC-3′;
Construct #2:
(SEQ ID NO: 17)
5′-CCGCAGCCATCAAGATGCTAAA-3′
and
(SEQ ID NO: 18)
5′-TTTAGCATCTTGATGGCTGCGT-3′;
Construct #3:
(SEQ ID NO: 19)
5′-ACCAGTGAGAATGTGACATTAA-3′
and
(SEQ ID NO: 20)
5′-TTAATGTCGCATTCTCACTGGG-3′.

Appropriate primers/oligonucleotides can be purchased and cloned into TMP following the protocol outlined by OpenBiosystem. Retrovirus harboring the shRNAmir sequence is prepared using the Pantropic Retroviral Expression System (Clontech).

The efficiency and doxycycline response of these three shRNAmir constructs can be tested in vitro using a 293-based cell line. This cell line will be transfected with pRevTet-Off to confer tTA expression. After antibiotic selection, this cell line expressing Tie-1 and tTA will be used to test the shRNAmir constructs. Briefly, cells are infected with the retrovirus encoding the shRNAmir at low MOI (e.g. 0.1) to promote single copy integration. Transduced cells express GFP and can be selected by FACS sorting. Doxycycline (1 μg/ml) is included after these procedures to suppress the expression of the shRNAmir. Responsiveness to induction is examined by titrating down the concentration of doxycycline in the growth medium (Dickins, Hemann et al., Nat Genet 37: 1289-1295, (2005)). These experimental conditions are used as general guidelines to characterize the Tie-1 shRNAmir constructs in vitro. The efficiency of Tie-1 knockdown is determined by western blotting using an anti Tie-1 antibody (Santa Cruz, C-18). The BglII/SphI fragment of the most efficient shRNAmir construct is excised and ligated into pLITMUS28i (NEB) together with a DNA duplex containing the following sequences: SphI-SV40 polyA signal-HindIII (FIG. 17C). The BglII/HindIII restriction fragment from this pLITMUS28i-based clone is used in transgenic mouse line construction. Restriction sites flanking the cassette are chosen carefully to minimize exogenous sequences at the ends of the cassette. After digestion with restriction enzymes, the expression cassette is excised in preparative scale by electro-elution prior to microinjection (Abbott, Mouse Genetics and Trangenics: A Practical Approach (2000)). Introduction of targeting constructs into pronuclear-stage zygotes and production of founders is performed by the Transgenic Core Facility. C57BL/6J is used as the host, because ApoE null mice in this background are much more susceptible to the development of atherosclerosis when compared to the ApoE null mice in the FVB/NJ background (Dansky et al., Arterioscler. Thromb. Vasc. Biol. 19: 1960-1968, (1999)) and has become the standard strain for studying atherosclerosis (Eitzman et al., Blood 96: 4212-4215, (2000)). PCR analysis is performed to screen for potential transgenic founders using genomic DNA isolated from tail biopsies (DNeasy Tissue Kit, Qiagen). “Tail tipping” will be done at weaning age (3 weeks). Once the transgenic lines are established, they are crossed to obtain dual heterozygote mice. Depending on the expression level of the transgene, analysis of the transgenic phenotype is done with the heterozygotes. This may alleviate the concern of insertional mutation of an endogenous gene during the transgenic construction (Abbott, Mouse Genetics and Trangenics: A Practical Approach (2000)). An alternative strategy for suppressing Tie-1 expression is to use Cre-Lox technology, which is more tedious but can give 100% Tie-1 knockdown.

Conditional suppression of Tie-1 expression in mice: Dual heterozygotes (CMV:tTA/TRE:Tie-1shRNAmir) are conceived and raised with Tie-1 endodomain expression suppressed by addition of doxycycline (200 μg/ml; Sigma) in the drinking water. Expression of the shRNAmir is induced by withdrawal of doxycycline when mice are 3 weeks old (weaning). A time course experiment is performed with doxycycline off up to 20 weeks. Expression of Tie-1 in the vasculature, especially at branch points, is examined by mRNA in situ hybridization. Once the expression knockdown of Tie-1 is confirmed to be correctly regulated temporally and the time course determined, the role of Tie-1 in the pathobiology of atherosclerosis can be ascertained. In addition, candidate Tie-1 inhibitor compounds can also be evaluated.

### Example 11

### Construction of a Mouse Line Expressing tTA Under the Murine Tie-2 Promoter (Mouse^{Tie-2:tTA})

The plasmid for creating this line is constructed as follows. The tTA coding sequence is excised from pRevTet-Off (Clontech) and used to replace the lacZ in pT2HlacZpA11.7 (a generous gift from Dr. Sato, Cornell U). The resultant plasmid harbors the following elements: a 2.1-kb murine Tie-1 promoter, tTA, pA, and a 10-kb Tie-2 promoter enhancer. This promoter/enhancer combination has been shown to express a transgene uniformly in the vasculature in both embryonic and adult mice (Schlaeger et al., Proc. Natl. Acad. Sci. 94: 3058-3063, (1997)).

Construction of mouse line expressing shRNAmir under TRE (mouse^{TRE:PAR1shRNAmir}): The strategy used in Example 10 is used to construct the transgenic line TRE:PAR1shRNAmir. Three constructs are made:

Construct #1:
(SEQ ID NO: 21)
5′-AGGCCAGCTGATGCCGAGTAAA-3′
and
(SEQ ID NO: 22)
5′-TTTACTCGGCATCAGCTGGCCG-3′;
Construct #2:
(SEQ ID NO: 23)
5′-AGGCCTTCTCCGCCATCTTCTT-3′
and
(SEQ ID NO: 24)
5′-AAGAAGATGGCGGAGAAGGCCG-3′;
Construct #3:
(SEQ ID NO: 25)
5′-CCCTGAATAACAGCATATACAA-3′
and
(SEQ ID NO: 26)
5′-TTGTATATGCTGTTATTCAGGT-3′.

Since mouse embryonic fibroblast 3T3 cells express endogenous PAR-1 (Marinissen et al., J. Biol. Chem. 278: 46814-46825, (2003)), the efficiency and doxycycline response of these shRNAmir constructs is tested in vitro using a 3T3-based cell line stably expressing tTA (3T3-tTA) (Clontech) using the strategy described above.

Once the transgenic mouse^{TRE:PAR1shRNAmir} is established, it is crossed with mouse^{Tie-2:tTA} to obtain the transgenic mouse^{Tie-2:tTA/TREPAR1shRNAmir}. Mice are conceived and raised with the shRNAmir expression suppressed by addition of doxycycline (200 μg/ml) in the drinking water. Conditional knockdown of PAR-1 in the endothelium is induced by withdrawal of doxycycline when mice are 3 weeks old (weaning) and a time course of PAR-1 knockdown is first determined with doxycycline off for 10 weeks at 1-week intervals. PAR-1 expression knockdown in endothelial cells is determined by immunostaining of sections of different organs with anti mouse PAR-1 antibody. The endothelium will be counterstained with anti PE-CAM-1 antibody. For evaluation of the role of PAR-1 or inhibitor compounds of the invention, the expression of shRNAmir (thus the knockdown of PAR-1 in the endothelium) is induced by doxycycline withdrawal. Mice will also be fed with a western type diet from this point onward. Experiments will last for 20 weeks. Mice will be sacrificed at weeks 5, 8, and 20. Experiments using ApoE null mice will be performed in parallel as controls. Aortic lipid accumulation and immunostaining of IP-10, IL-6, G-CSF, VCAM-1, and ICAM-1 is performed as described above.

### Example 12

### Assays to Evaluate the Involvement of VEGFR2 in Thrombin-Induced Endothelial Cell Inflammation

Three siRNA duplexes specific for human VEGFR-2 (NM_{—}002253) can be purchased from Ambion (#220-222). Delivery of siRNA oligos into endothelial cells is achieved using SilentFect (Bio-Rad) as described (Parikh, Mammoto et al., PLoS Med 3: e46, (2006)). Efficiency of expression knockdown of VEGFR2 is determined by real-time PCR analysis. Once the efficiency of the VEGFR2 siRNA is established, the contribution of VEGFR2 in thrombin-induced permeability and gap formation can be determined using the assays described above. In addition the contribution of VEGFR2 for thrombin-induced cytokine upregulation in endothelial cells can also be determined using HUVECs transfected with either the control siRNA or VEGFR2-siRNA and adding thrombin to 1 U/ml. The supernatant is harvested and analyzed using an antibody array such as the Human Inflammation Antibody Array 3.1 from RayBio (#H0129803). This antibody array allows simultaneous examination of 40 different cytokines. The results from such antibody array experiment will be validated by real-time PCR.

The VEGFR inhibitor, SU5416 can also be used to further assess the role of VEGFR2 in atherosclerosis. In one group, 50 μl of SU5416 in DMSO will be administrated into the mice by intra-peritoneal injection twice weekly (50 mg/kg). This dose is chosen because SU5416 effectively blocks tumor progression in several xenograph models using this regimen of injection (Mendel, Schreck et al., Clin Cancer Res 6: 4848-4858, (2000)). 50 μl of DMSO will be injected into mice of the second group under the same regimen. After 8 weeks, mice will be sacrificed and the development of atherosclerotic lesion in the SU5416 treated mice will be examined and compared to that in the control DMSO injected group as described above. This model can also be used to test additional VEGFR inhibitors as candidate therapeutic inhibitor compounds of the invention.

### Example 13

### Tie-1 Overexpression in Endothelial Cells Induces Proinflammatory Responses In Vitro

In the experiments described below, we have found that when overexpressed in endothelial cells in vitro, Tie-1 is specifically tyrosine phosphorylated. We have also found that Tie-1 upregulates inflammatory markers IP-10, IL-6, CCL2, VCAM-1, E-selectin, and ICAM-1, through a p38-dependent mechanism. Additionally, attachment of cells of monocytic lineage to endothelial cells is also enhanced by Tie-1 expression. Collectively, our data show that Tie-1 has a proinflammatory property and plays a role in the development of vascular inflammatory diseases such as atherosclerosis.

### Methods

Adenovirus construction: For adenovirus production, we chose the AdEasy adenoviral expression system due to its ease of use and ability to generate high titer virus. Coding sequence of mouse Tie-1 excluding the endogenous leader sequence was amplified from cDNA prepared from mouse lung tissue. The following expression cassette was constructed: IgK leader sequence, a Flag epitope tag sequence, and the coding region of mouse Tie-1. This cassette was subcloned into the shuttle vector pAdTrack, which contains two CMV promoters to express the gene of interest and the green fluorescent protein (GFP) independently. After homologous recombination in the E. coli strain BJ5183 (Stratagene), the recombined pAdeasy DNA (PacI-cut) was used to transfect 293A cells (Invitrogen). After several rounds of amplification, the virus was purified using the Ad-Pure Adenoviral Purification Kit (BioVintage) and tittered using 293A as the host. Immediately prior to use, the virus was desalted and buffer-exchanged into endothelial growth medium using a spin column (Pierce).
Adenoviral infection: Human umbilical vein endothelial cells (HUVECs) and human aortic endothelial cells (HAECs) were purchased from Cambrex and maintained as recommended by the manufacturer. To overexpress Tie-1, 200,000 endothelial cells were plated in one well of a E-well plate. After overnight incubation, adenovirus harboring the Tie-1 expression cassette was added to the endothelial cells at MOI of 10:1. An “empty virus” expressing only GFP was used as a control. Seventeen hours later, medium was replaced with fresh medium. Cells were maintained and processed at the desired time points for the experiments described below.
Tie-1 tyrosine phosphorylation status: Seventeen hours post infection, cells were treated with 1 mM sodium orthovanadate for 45 mins and lysed with RIPA buffer supplemented with 1× complete protease inhibitor (Roche), 2 mM sodium orthovanadate, 1 mM NaF, 2.5 mM b glycerol phosphate, 2.5 mM sodium pyrophosphate, 0.0045% (v/v) hydrogen peroxide, and 1 mM EDTA. Lysates were immunoprecipitated with an anti-Tiel antibody (Santa Cruz, SC-342). Western blotting with anti phosphotyrosine antibody (4G10, Upstate) was used to determine the phosphorylation status of Tie-1. The membrane was stripped and reblotted with the anti-Tiel antibody. A portion of the lysates was immunoprecipitated with 4G10. Tyrosine phosphorylation of Tie-1 was confirmed by western blotting with the anti-Tiel antibody using these 4G10 immunoprecipitates.
Antibody array experiment: HUVECs were infected as described. Forty-eight hours later, supernatants from Tie-1 expressing or GFP expressing cells were collected. After brief centrifugation to remove cell debris, the supernatants were spun through a 0.22-μm cellulose acetate filter (Spin-X, Costar). Cytokine and chemokine contents in the conditioned media were screened using the RayBio Human Inflammation Antibody Array 3.1(RayBiotech).
Real-time polymerase chain reactions (PCR)—Total RNAs from endothelial cells were isolated by the RNAeasy Mini Kit (Qiagen) and used as templates in oligo-dT primed reverse transcription using the Superscript III reverse transcriptase (Invitrogen). Real-time PCRs were performed using the QuantiTect Probe PCR Kit (Qiagen) with the 7500 Real Time PCR System (Applied Biosystems). Gene of interest and GAPDH were multiplexed for normalization as described (Dupuy et al., Exp. Cell Res. 185: 363-372., (1989)). The following real time PCR probes were purchased from Applied Biosystems and used in this study: CCL2 5′-GATGCTGAAAAATGGCAAATCCAAC-3′(SEQ ID NO: 27); VCAM-1: 5′-TGATGTTCAAGGAAGAGAAAACAAC-3 (SEQ ID NO: 28)′; ICAM: 5′-GGGGCTCTGTTCCCAGGACCTGGCA-3′(SEQ ID NO: 29); CXCL10: 5′-GTGGCATTCAAGGAGTACCTCTCTC-3′(SEQ ID NO: 30); E-selectin: 5′-GTGTGAGCAAATTGTGAACTGTACA-3′(SEQ ID NO: 31); IL-6: 5′-GGATTCAATGAGGAGACTTGCCTGG-3′ (SEQ ID NO: 32).
ELISA—HUVECs were infected as described above. Supernatants were collected from Tie-1 expressing or GFP expressing cells 70 hours post infection. The ELISA kit for human IL-6 was purchased from R and D Systems.
Western blots—Endothelial cell lysates were prepared in denaturing SDS-PAGE loading buffer, sonicated, heated at 80° C. for 5 mins, and fractionated on SDS-PAGE. After transferring to a PVDF membrane, expression of Tie-1 (Santa Cruz), VCAM-1 (Santa Cruz), ICAM-1 (Santa Cruz), and GAPDH (Chemicon) were determined by western blotting.
p38 inhibition study—HUVECs were infected with either GFP- or Tie-1 adenovirus as described above. DMSO or SB-203580 (p38 specific inhibitor, Calbiochem) was also added to the medium. 18 hours later, cells were incubated with fresh medium with either DMSO or SB-203580. Total RNAs were harvested 48 hours post infection and used in real-time PCR analysis.
U937/HAEC adhesion assay—A published procedure was followed with modifications (Luscinskas et al., J. Cell Biol. 125: 1417-1427, (1994)). HAECs were infected with either GFP or Tie-1 adenovirus as described above. 48 hours post infection, 1×10⁶ U937 cells (ATCC) were added. Cells were incubated at room temperature under rotation (64 rpm) for 30 mins. Cells were then incubated statically at 37° C. for 30 mins. Unattached U937 cells were washed off with growth medium. Cells were then fixed in 4% PFA in PBS for 15 mins.

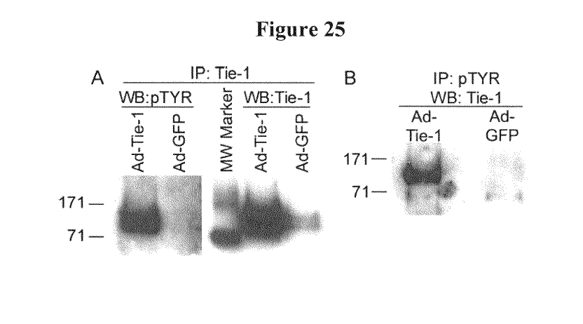

Results: In order to investigate the role of Tie-1 in endothelial inflammation, we overexpressed full-length mouse Tie-1 in human endothelial cells in vitro. We examined the phosphorylation status of Tie-1 when overexpressed in HUVECs. Tie-1 was overexpressed by adenoviral infection. As a control, cells were infected with a GFP-producing adenovirus. As shown in FIGS. 25A and 25B, overexpression of Tie-1 in HUVECs led to tyrosine phosphorylation of the receptor kinase. This activation was achieved presumably due to receptor clustering resulting from the high protein level. Endogenous Tie-1 was not tyrosine phosphorylated. To our knowledge, this is the first documentation of Tie-1 autophosphorylation when overexpressed in endothelial cells.

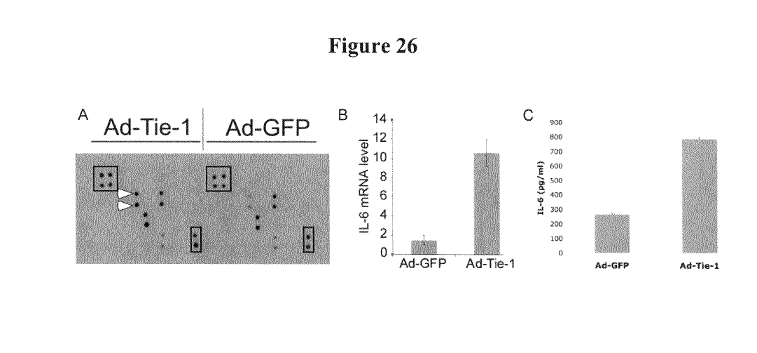

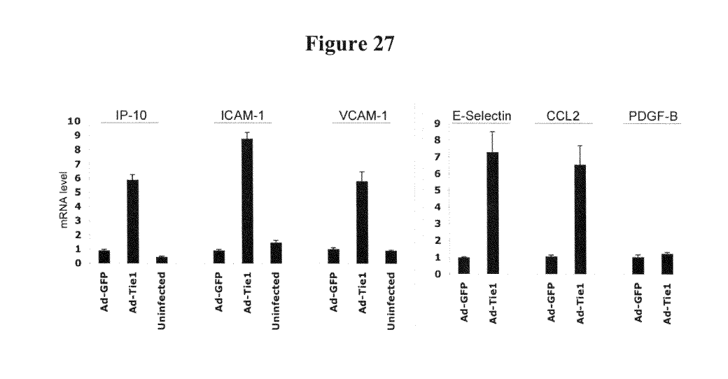

Next, conditioned medium from HUVECs infected with either GFP or Tie-1 adenovirus was used in an antibody array experiment designed to screen for inflammatory cytokines/chemokines. We detected upregulation of IL-6 in HUVECs when Tie-1 was overexpressed (FIG. 26A). This result was validated by both real-time PCR and EILSA assays (FIGS. 26B and 26C). We also screened other inflammatory markers by real-time PCR and found that interferon-inducible protein-10 (IP-10), CCL2 (also called monocyte chemoattractant protein-1 or MCP-1), ICAM-1, VCAM-1, and E-selectin were upregulated by Tie-1, whereas PDGF-B was not induced (FIG. 27).

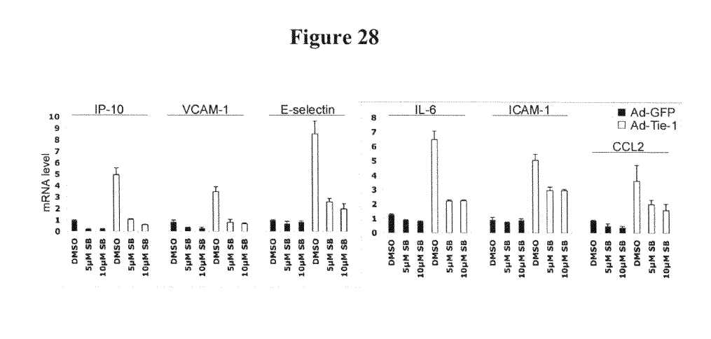

Since it has been reported that the p38 pathway is critical for inducible expression of IP-10, VCAM-1, and ICAM-1 (Rahman et al., Mol. Cell. Biol. 21: 5554-5565, (2001); Minami et al., J. Biol. Chem. 278: 6976-6984, (2003); Sheng et al., J Leukoc Biol 78: 1233-1241, (2005); Wong et al., Clin. Exp. Immunol. 139: 90-100, (2005); Wong et al., Allergy 61: 289-297, (2006)), we investigated the requirement of p38 activation in Tie-1-induced endothelial inflammation. As shown in FIG. 28, SB-203580 almost completely blocked Tie-1-induced upregulation of IP-10, VCAM-1, E-selectin, and IL-6. Significant inhibition of ICAM-1 and CCL2 upregulation was also observed. Therefore, Tie-1 induces endothelial inflammation through p38 activity.

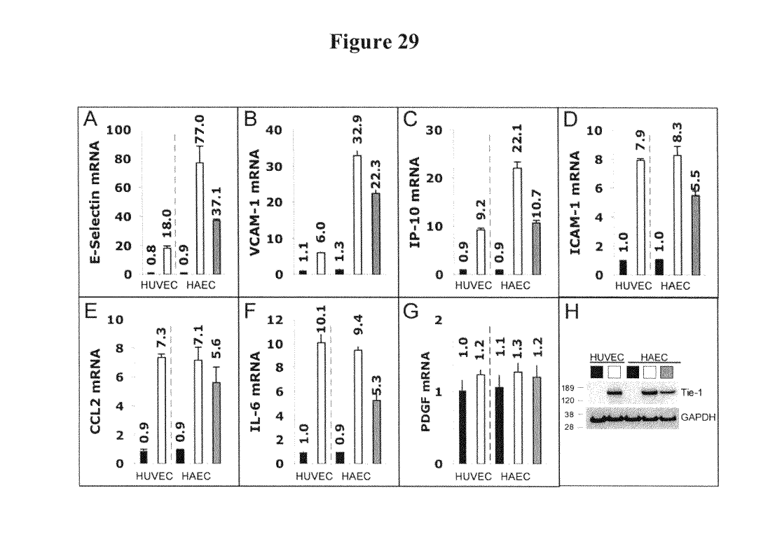

We compared HUVECs with human aortic endothelial cells (HAECs) with respect to Tie-1-induced inflammation. As shown in FIG. 29A-29C, at 48 hrs, Tie-1-induced upregulation of VCAM-1, E-selectin, and IP-10 was significantly higher in HAECs than in HUVECs. Note that upregulation of E-selectin, VCAM-1, and IP-10 was already the same or higher in HAECs infected with half the amount of Tie-1 adenovirus than those in HUVECs infected with full amount of virus (FIGS. 29A-29C, compare open and gray bars). Upregulation of IL-6, CCL2, and ICAM-1 was comparable in both cell types (FIGS. 29D-29F). PDGF-B was not upregulated in either cell type (FIG. 29G).

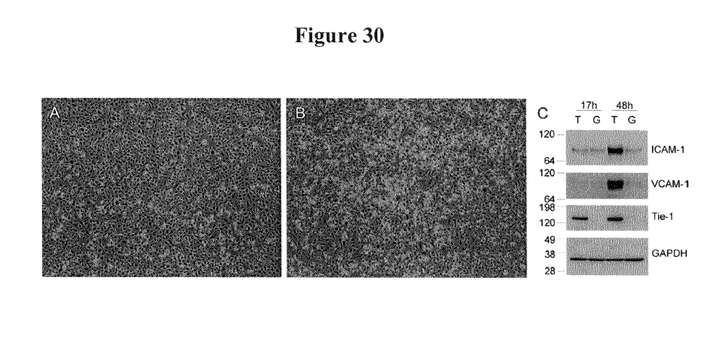

We have shown that expression of Tie-1 in endothelial upregulates expression of adhesion molecules ICAM-1, VCAM-1, and E-selectin by real time PCR analysis. Therefore, we tested whether overexpression of Tie-1 in endothelial cells in vitro would promote monocyte attachment in vitro. As shown in FIG. 30A-30C, expression of Tie-1 in HAECs significantly promoted attachment of U937 cells to the endothelial cells. This result is consistent with our observation that adhesion molecules are upregulated in endothelial cells when Tie-1 is overexpressed.

In the experiments described above, we show that inflammatory markers such as IP-10, IL-6, and CCL2, VCAM-1, ICAM-1, and E-selectin are upregulated when Tie-1 is overexpressed in endothelial cells. We further show that several proinflammatory responses-namely upregulation of IP-10, VCAM-1, and E-selectin-are more pronounced in endothelial cells of aortic origin. In addition, attachment of U937 cells to HAECs is enhanced by Tie-1 overexpression.

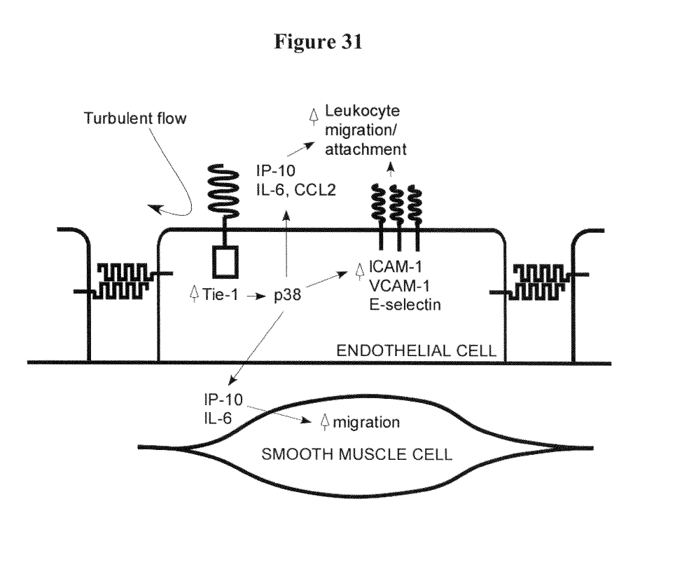

Our findings are particularly relevant to atherosclerosis development. For example, injections of exogenous IL-6 significantly enhanced early development of atherosclerosis in both C57B1/6 mice and an atherosclerosis-prone mouse line (ApoE null). IP-10 has been shown to be upregulated in endothelial cells in atherosclerotic lesions and is a chemotactic and mitogenic factor for smooth muscle cells. In addition, IP-10 is also a potent chemoattractant for monocytes and activated T-lymphocytes. Importantly, atherosclerosis development was significantly inhibited in IP10^{-/-}/ApoE^{-/-} double knockouts compared to control ApoE^{-/-} mice. Anti-CCL2 (MCP-1) gene therapy significantly inhibits atherosclerosis development and progression in ApoE mice^{-/-}. Adhesion molecules ICAM-1, VCAM-1, and E-selectin have all been shown to play a critical role in atherogenesis (Nageh et al., Arterioscler. Thromb. Vasc. Biol. 17: 1517-1520, (1997); Nakashima et al., Arterioscler. Thromb. Vasc. Biol. 18: 842-851, (1998); Collins et al., J. Exp. Med. 191: 189-194, (2000); Cybulsky et al., J. Clin. Invest. 107: 1255-1262, (2001)). FIG. 31 provides our working hypothesis in how Tie-1 may play a role in atherosclerosis development based on our data. At arterial branch points, endothelial cells experience unusually high turbulent flow. This hemodynamic condition upregulates Tie-1 expression and activities. Proinflammatory molecules such as IP-10, IL-6, CCL2, E-selectin, ICAM-1, and VCAM-1 are subsequently induced through a p38-dependent mechanism. These events lead to recruitment and attachment of leukocytes. Proinflammatory cytokines/chemokines such as IP-10 and IL-6 may also activate smooth muscle cell to migrate to the intima and to proliferate. These molecular and cellular events collectively may correspond to the initial stage of atherosclerosis development.

### Example 14

### Thrombin Transactivates Multiple Receptor Tyrosine Kinases in Endothelial Cells

Thrombin is a multifunctional serine protease that plays a critical role in endothelial biology. The principal receptors for thrombin belong to a class of receptors known as the protease-activated receptors (PARs). Four PARs have been identified to date. PAR-1, 2, and 3 are expressed on human endothelial cells. Amongst these three receptors, thrombin specifically activates PAR-1 and PAR-3. Thrombin activates these receptors by cleavage at the N terminus of the receptor, which acts as its own ligand to signal. Activation of PARs by thrombin initiates a complex network of intracellular signals including protein kinase C, PI3 kinase, Src, MAP kinases, Rho kinase, and those proteins discovered in the experiments described in the Examples, above.

We sought to identify the downstream effectors of thrombin that may be involved in the thrombin-mediated proinflammatory response in endothelial cells. We hypothesized that thrombin achieves its diverse cellular responses in endothelial cells by transactivating multiple receptor tyrosine kinases.

Methods: Confluent HUVECs/RCC7 were pretreated with 1 mM sodium orthovanadate for 15 mins and stimulated with α-thrombin (Calbiochem) at 5 U/ml for 30 mins. Lysates were prepared according to the protocol included in the Phospho-RTK Array kit (R&D Systems).

Results: We used a phospho receptor tyrosine kinase antibody array to survey transactivation of receptor tyrosine kinases in HUVECs upon thrombin stimulation. In this assay, antibodies against the extracellular domain of 42 receptor tyrosine kinases were spotted on a membrane in duplicate. These antibodies captured the cognate receptors from the lysate. Activation status was evaluated using an anti phospho-tyrosine antibody conjugated to HRP. Therefore, if a receptor is expressed in HUVECs and is phosphorylated, two spots would appear at a specific location on the membrane upon chemiluminescence detection.

As shown in FIG. 7A, thrombin treatment induced significant phosphorylation of 12 receptor tyrosine kinases. They included EphA2, EGFR, insulin receptor, IGF-IR, AXL, HGFR (c-met), Flt-1, KDR, c-RET, MER, Tie-1, and Tie-2. Receptor tyrosine kinases that were probed but were either not phosphorylated or not expressed in HUVECs included ErbB2, ErbB3, ErbB4, FGF R1, FGF R2α, FGF R3, FGF R4, Dtk, MSP R, PDGF Rα, PDGF Rβ, SCF R (c-kit), Flt-3, M-CSF R, ROR1, ROR2, TrkA, TrkB, TrkC, VEGF R3, MuSK, EphA1, EphA3, EphA4, EphA6, EpHA7, EphB1, EphB2, EphB4, and EphB6. When the same experiment was repeated with an epithelial cancer cell line (RCC4), only EGFR was significantly transactivated (FIG. 7B). Thus, the extensive cross talk between the thrombin receptor and receptor tyrosine kinases may be unique to endothelial cells.

### Example 15

### Thrombin Induces Time-Dependent Tyrosine Phosphorylation of EphA2 in HUVECs

Having shown in Examples 1-14, above, that thrombin induces extensive cross-talk among 12 RTKs, we chose to focus on EphA2. Ephrins and Eph receptors have been implicated in to be important in vascular function, endothelial cell cancers and tumorigenesis, and in some inflammatory disorders such as rheumatoid arthritis. Ephrin-A1 was first identified as an immediately-early response gene of endothelial cells induced by inflammatory stimuli such as TNF-α, IL-1β, and lipopolysaccharide. Ephrin receptors, including EphA2, are shown to be upregulated during inflammation. For example, EphB/EphrinB system appears to play a role in the inflammatory responses in rheumatoid arthritis. However, it is worth noting that these EphB/EphrinB proteins were not identified as phosphorylated or expressed in the assay described above. Other than attribution of EphA2 being a mediator of TNF-a-induced angiogenesis in micro-pocket corneal assays in mice, very little is known about the specific functions of these Eph receptors/Ephrins in endothelial inflammation and the role of EphA2 in thrombin biology and endothelial inflammation has not been suggested to date.

Methods: To establish a time course of tyrosine phosphorylation of EphA2 by thrombin, a published protocol developed to detect tyrosine phosphorylation of VE-cadherin-associated proteins induced by thrombin in endothelial cells was used (Ukropec et al., J. Biol. Chem. 275: 5983-5986, (2000)). Confluent HUVECs were stimulated with thrombin (1 U/ml) for the desired amount of time. Clarified lysates were immunoprecipitated with 2 μg of a polyclonal anti human EphA2 antibody (R&D Systems) and Protein A/G Plus. Tyrosine phosphorylation was detected by western blot using the 4G10 antibody. The membrane was stripped and reblotted with an rabbit polyclonal antibody against EphA2 (Santa Cruz).

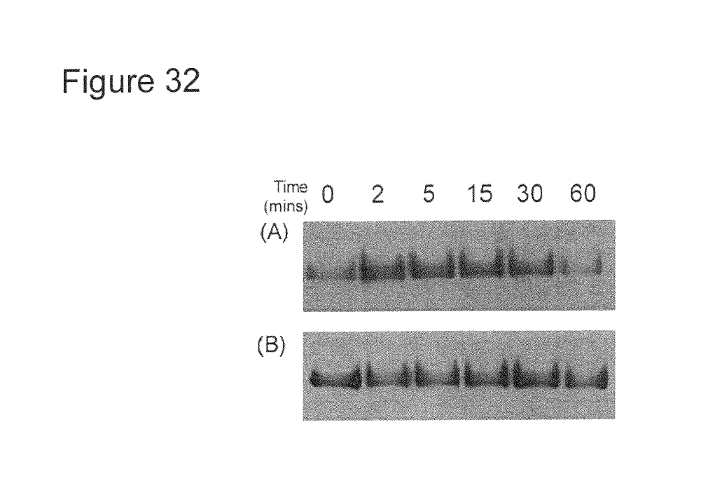

Results: FIGS. 32A-32B illustrate the time course of EphA2 phosphorylation induced by thrombin. Tyrosine phosphorylation of EphA2 was detected as early as 2 minutes and lasted to at least 30 minutes post thrombin-stimulation. Therefore, this result was a validation of transactivation of EphA2 by thrombin determined by the antibody array in FIGS. 7A-7B.

### Example 16

### Thrombin-Induced ICAM-1 Upregulation in HUVECs Requires EphA2

In this example, we sought to identify the function of EphA2 activation by thrombin in endothelial cells by using siRNA knockdown technology. Because thrombin potently upregulates ICAM-1 expression in endothelial cells, we opted to determine whether EphA2 was involved in this regulation.

Methods: Two validated Stealth siRNA duplexes specific to human EphA2 and a negative control duplexes were purchased from Invitrogen (EphA2 siRNA, catalog number: 12938-022, sequence: 5′-gca agg aag ugg uac ugc ugg acu u-3′ (siRNA #1, SEQ NO ID 33) and control siRNA, catalog number 12935-300, sequence: 5-ggg acc uga ugc aga aca uca uga a-3 (siRNA #2, SEQ NO ID 34)). A mixture of siRNA (0.2 nmol) and Silentfect (4 μl, BioRad) was prepared in 500 μl serum-free EBM2 basal medium. After incubation at room temperature for 20 mins, the mixture was added to 80%-confluent HUVECs in a 10-cm plate in the presence of 5 ml fresh EBM2-MV medium. Sixteen hours later, cells were split and seeded into 6-well plates. Next day, the confluent HUVECs were stimulated with thrombin (1 U/ml) in 2 ml EBM-2 supplemented with 0.5% FBS for 6 hours. Experiments were terminated with lysis of cells using 300 μl 2×SDS PAGE loading buffer. ICAM-1 protein expression were detected by western blots using an anti ICAM-1 antibodies. The following protocol was used to stably express mouse EphA2 in HUVEC. The coding region of mouse EphA2 was amplified from cDNAs prepared from 4T1 cells and cloned into a pLNCX2-based retroviral vector, in which an IRES-EGFP cassette was inserted downstream of mouse EphA2. To generate retrovirus, either IRES-EGFP-alone or EphA2-IRES-EGFP plasmid was transfected into Phoenix-Ampo cells using Lipofectamine 2000 (Invitrogen). Medium was replaced with serum-free DMEM supplemented with 10 ng/ml bFGF and 200 ng/ml EGF 16-hours post transfection. After an additional 24 hours, supernatant was collected and used to infect HUVECs in the presence of 40 μg/ml protamine sulfate. Transduced cells were selected and expanded in the presence of 0.8 mg/ml G418.

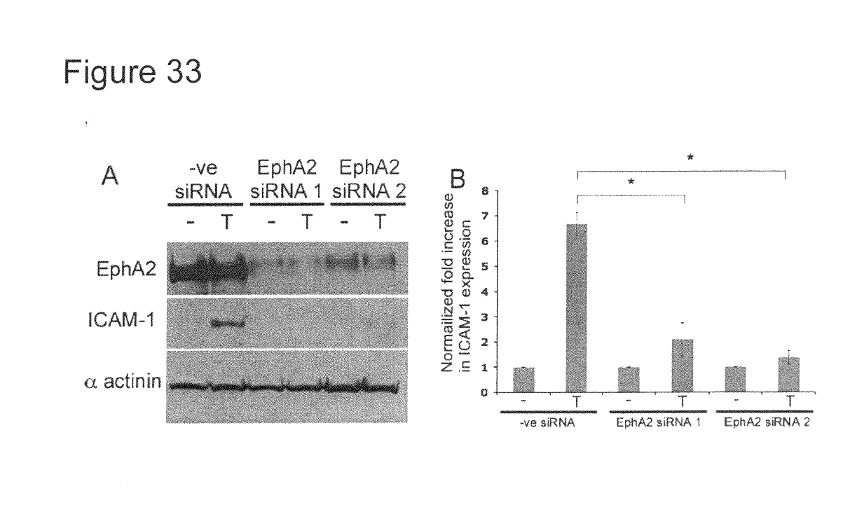

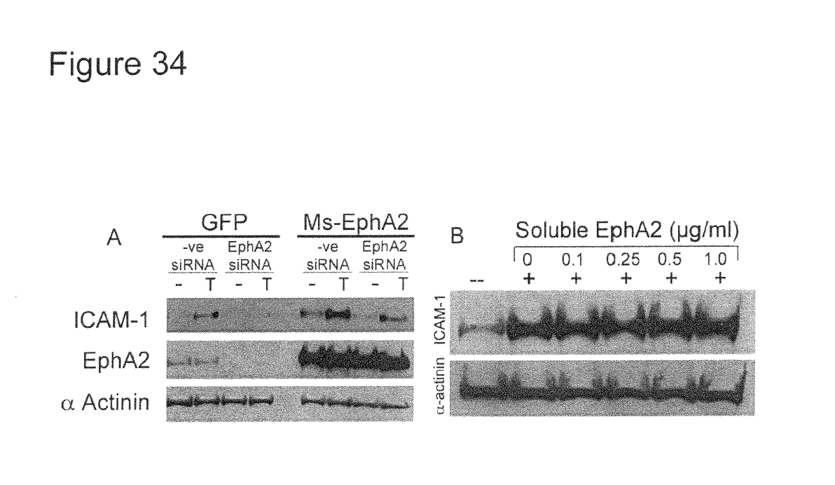

Results: As shown in FIGS. 33A-33B, transient transfection of each of these two siRNA duplexes significantly reduced EphA2 protein expression in HUVECs. Transfection of a control siRNA duplex had no effect on EphA2 expression. Thrombin significantly upregulated ICAM-1 expression in HUVECs treated with the control siRNA but failed to induce ICAM-1 expression when EphA2 was knockdown by either EphA2-specific siRNA duplex. To further corroborate the involvement of EphA2 in ICAM-1 upregulation by thrombin, we performed expression rescue experiments. HUVECs were transduced by either GFP- or mouse-EphA2 retrovirus. Infected cells were selected and expanded in the presence of G418. Retrovirus infection and G418 selection had no effects on thrombin-induced, EphA2-mediated ICAM-1 upregulation (FIG. 34A, left). Expression knockdown of endogenous EphA2 by siRNA again blocked ICAM-1 expression after thrombin stimulation in the GFP-virus infected group. However, in the presence of exogenously expressed mouse EphA2, thrombin-induced ICAM-1 upregulation was significantly restored even when endogenous human EphA2 was knockdown by siRNA. Collectively, our results strongly suggest that EphA2 is a downstream effector of thrombin stimulation and is critical in upregulation of ICAM-1 in endothelial cells. To exclude the possibility that induction of ICAM-1 expression upon thrombin stimulation was due to EphA2 interactions with ephrins, we added soluble EphA2 in excess during thrombin stimulation. As shown in FIG. 34B, co-incubation of soluble EphA2 at concentrations up to 1 μg/ml failed to block ICAM-1 expression induced by thrombin. Consistent with this result, addition of EphrinA1-FC up to 10 μg/ml did not stimulate ICAM-1 expression.

### Example 17

### Suppression of EphA2 Blocks Monocyte Attachment to Thrombin-Stimulated HUVECs

One of the consequences of thrombin-induced ICAM-1 upregulation is enhanced attachment of leukocytes to the endothelium. Since suppression of EphA2 blocked ICAM-1 upregulation induced by thrombin, we reasoned that it would also block leukocyte attachment to stimulated HUVE monolayer. Therefore, we tested the ability of U937, a monocytic cell line, to attach to thrombin-treated HUVECs in the presence or absence of EphA2. In this experiment, both GFP-expressing and mouse-EphA2-expressing HUVECs were used.

Methods: A published procedure was followed with modifications (Luscinskas, Kans. et al., J. Cell Biol. 125: 1417-1427, (1994)). Confluent HUVECs in a 24-well plate were stimulated with thrombin (5 U/ml) in 2 ml EBM-2 supplemented with 0.5% FBS for 6 hours. Meanwhile, U937 were labeled with 0.1 μM Cell Tracker Red CMTPX (Molecular Probes) in PBS for 10 mins, followed by a 6-hour recovery period in 10% FBS/RIPM. At the end of the 6-hour thrombin stimulation, labeled U937 cells were incubated with HUVECs at room temperature under rotation for 60 mins. Unattached U937 cells were washed off with growth medium. Cells were then fixed in 4% PFA in PBS for 15 mins prior to visualization by fluorescence microscopy.

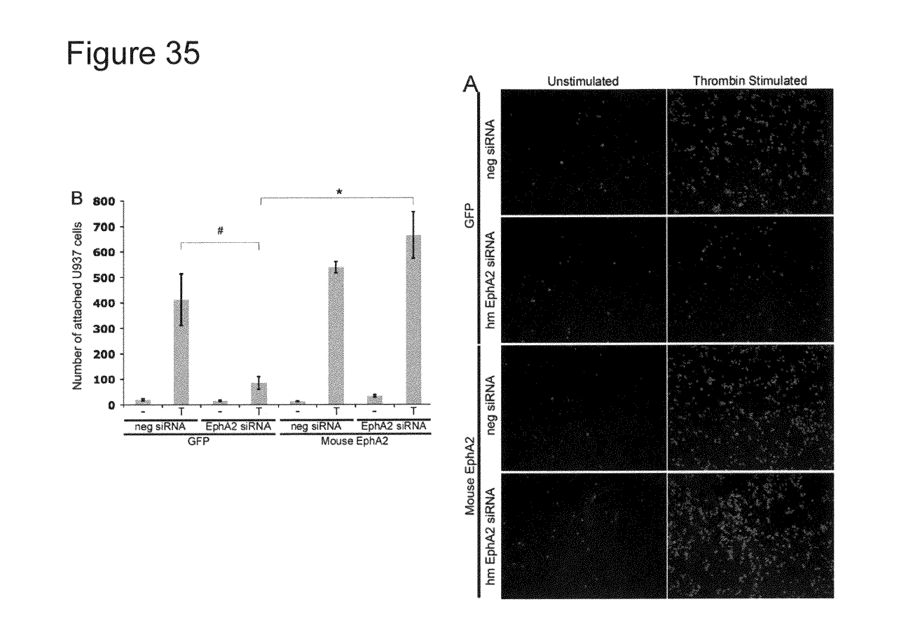

Results: Thrombin potently induced U937 attachment to HUVECs when cells were treated with the non-specific control siRNA, both in the GFP- and mouse-EphA2 expressing HUVECs (FIGS. 35A-35B). However, U937 attachment to thrombin-stimulated HUVECs was significantly blocked when endogenous EphA2 was knocked down by siRNA. In contrast, overexpression of mouse EphA2 in HUVECs with endogenous EphA2 suppressed restored thrombin-induced U937 attachment. These results were consistent with our observation that EphA2 is required for ICAM-1 upregulation induced by thrombin.

### Example 18

### Thrombin and EphrinA1 Activate EphA2 Via Two Distinct Mechanisms

One of the cognate ligands for EphA2 is EphrinA1. It has been demonstrated that EphrinA1 mediates TNF-a-induced angiogenesis in micro-pocket corneal assays in mice. Therefore, we sought to determine whether stimulation of HUVECs with EphrinA1 alone was sufficient to induce ICAM-1 expression. Furthermore, we sought to identify the mechanisms by with thrombin and EphrinA 1 induce EphA2 activation.

Methods: Mouse EphrinA1 in a form of FC fusion was purchased from R and D Systems. Pharmacological agents PP2 was used to examine the role of Src in EphA2 phosphorylation. Confluent HUVECs were pretreated with PP2 (2 μM) or DMSO for 1 hour, followed by a 10-min treatment with sodium orthovandate (1 mM). Cells were then stimulated for 30 mins with either 1 U/ml thrombin or 250 ng/ml EphrinA2-FC. HUVECs were lysed in RIPA buffer. Clarified lysates were immunoprecipitated with 2 μg of a polyclonal anti human EphA2 antibody (Santa Cruz) and Protein A/G Plus. Tyrosine phosphorylation and EphA2 were detected using the 4G10 and the EphA2 antibody (Santa Cruz), respectively.

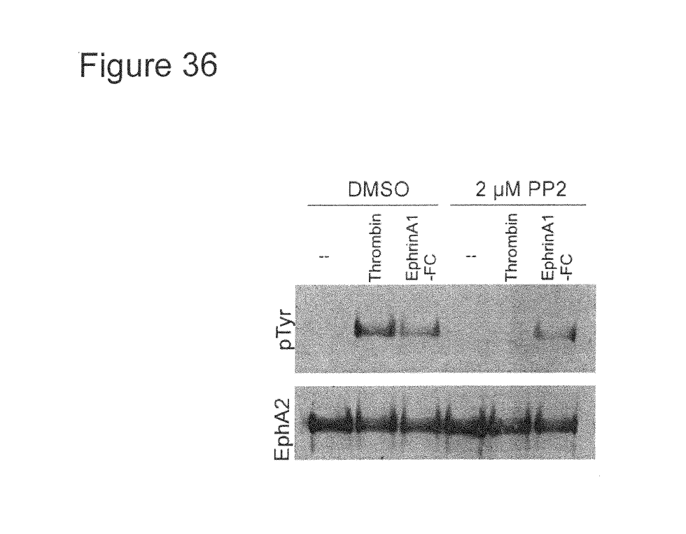

Results: As shown in FIG. 36, thrombin-induced EphA2 tyrosine phosphorylation was completely abrogated by PP2. Our results suggest that thrombin causes EphA2 activation via a Src-family kinase. Next, we contrasted the activation mechanisms of EphA2 by thrombin and the canonical ligand EphrinA1. As expected, EphrinA1, when presented as a FC-fusion protein, induced significant activation of EphA2 in endothelial cells, as did thrombin. However, in contrast to thrombin stimulation, tyrosine phosphorylation of EphA2 by its cognate ligand EphrinA1 was insensitive to PP2 treatment. This observation suggests that thrombin and EphrinA1 induced EphA2 phosphorylation via a Src-dependent and Src-independent pathway, respectively. Therefore, our results suggest that EphA2 can be activated by two distinct mechanisms, each may produce different phenotypes in endothelial cells.

### Example 19

### Thrombin Signals Through PAR-1 to Transactivate EphA2

Thrombin activates PAR-1, -3, and -4. Each activated receptor may transmit a unique set of signals. Therefore, we employed agonistic peptides specific to PAR-1, -2, and -4 to determine which receptor is involved in EphA2 transactivation. The requirements of G proteins in EphA2 transactivation was also examined using pertussis toxin.

Methods: HUVECs were prepared and stimulated as described in above except that the following peptides at 20 μM were used as stimulants: TFLLR-NH₂ (PAR-1; SEQ ID NO: 11), RLLFT-NH₂ (negative control for PAR-1; SEQ ID NO: 12), SLIGKV-NH₂ (PAR-2; SEQ ID NO: 13), and GYPGKF-NH₂ (PAR-4; SEQ ID NO: 14).

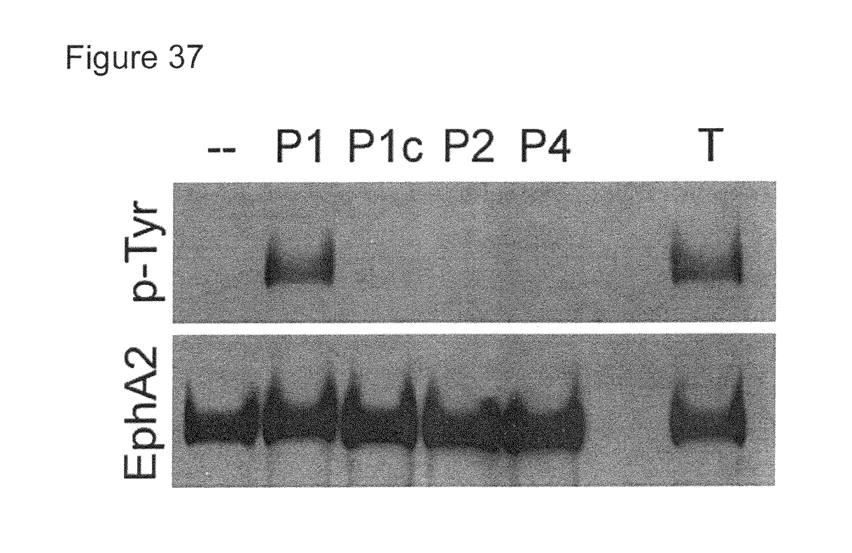

Results: As shown in FIG. 37, activation HUVECs with PAR-1 specific agonistic peptide recapitulated EphA2 transactivation seen by thrombin treatment. A control PAR-1 peptide with a reverse sequence, PAR-2, and PAR-4 specific agonist failed to induce EphA2 tyrosine phosphorylation at the same concentration. These results strongly suggest that thrombin transactivates EphA2 through PAR-1 in HUVECs.

### Example 20

### Thrombin-Activated EphA2 Transduces Signals in Endothelial Cells

Many receptor tyrosine kinases associate with SH2-containing signaling molecules at phosphorylated tyrosine sites. Since EphA2 is heavily tyrosine phosphorylated in response to thrombin stimulation, we sought to determine whether this phosphorylation event is functional in terms of relaying intracellular signals.

Methods: We used a phosphotyrosine profiling array to identify signaling pathways linked to EphA2 activation by thrombin. HUVECs were prepared and stimulated with 1 U/ml thrombin for 5 mins. TranSignal Phosphotyrosine Profiling Array (Panomics) was used according to the manufacturer's protocol, except that a biotinylated anti-human EphA2 (R & D) was used as the detection antibody.

Results: Confluent HUVECs were stimulated with thrombin for 5 mins to induce EphA2 phosphorylation. This stimulated lysate and an unstimulated lysate were used in the antibody array experiment. The concept of this array is similar to a far-western blot. On this array, the SH2 domains of 38 signaling molecules were spotted in duplicate. Upon incubation with a lysate, each of these SH2 domains will bind to its specific, tyrosine phosphorylated protein targets. When an anti-human EphA2 antibody was used for detection, any SH2 domains that are bound to EphA2 will appear as two dots in a specific location of the membrane. Therefore, pathways activated post EphA2 phosphorylation can be identified.

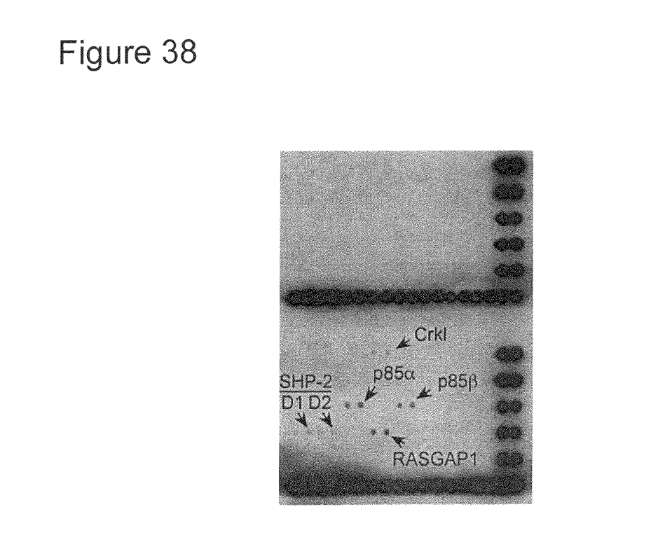

As shown in FIG. 38, thrombin-activated EphA2 associated with the SH2 domains of Crk1, the α and β regulatory subunits of PI3 kinase, non-receptor tyrosine phosphatase SHP-2 (D1 and D2 represent the first and second SH2 domains of SHP-2), and RASGAP1. Other SH2-domain proteins that were probed but were found not to be associating with EphA2 included Abl1, Brdg1, Btk, Csk, Eat2, Fes, Fgr, Fyn, Grap, Grb2, Grb14, Hck, Lck, Lyn, Matk, Nck1, Nck2, PLC-γ, RaLP, SHC1, SHC2, SHC3, Src, Stap2, TNS, Yes, and Zap70. These results suggest that thrombin activation of EphA2 has signaling consequences.

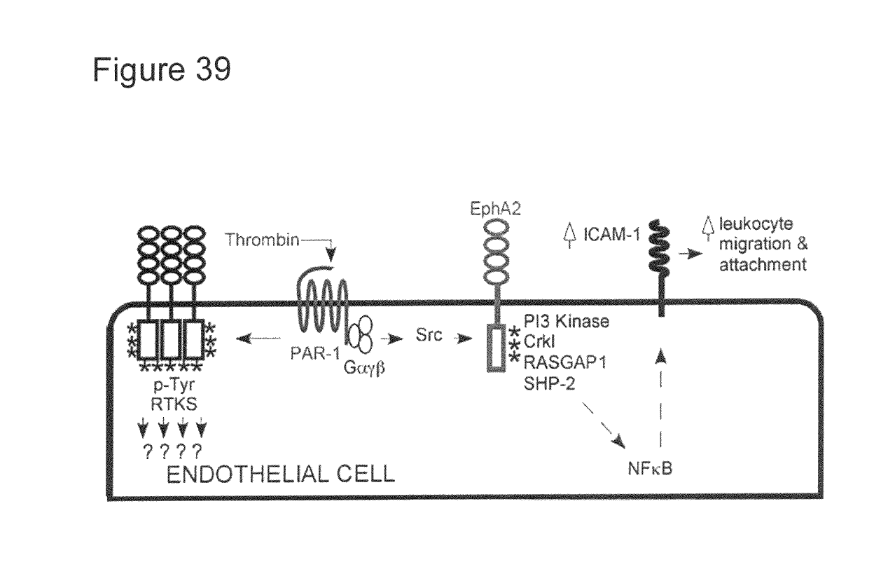

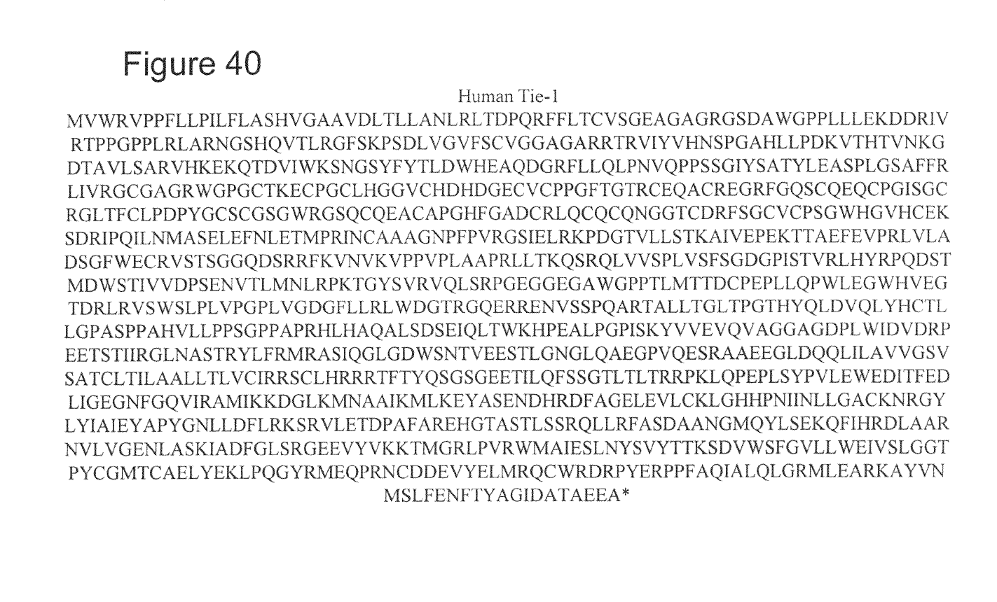

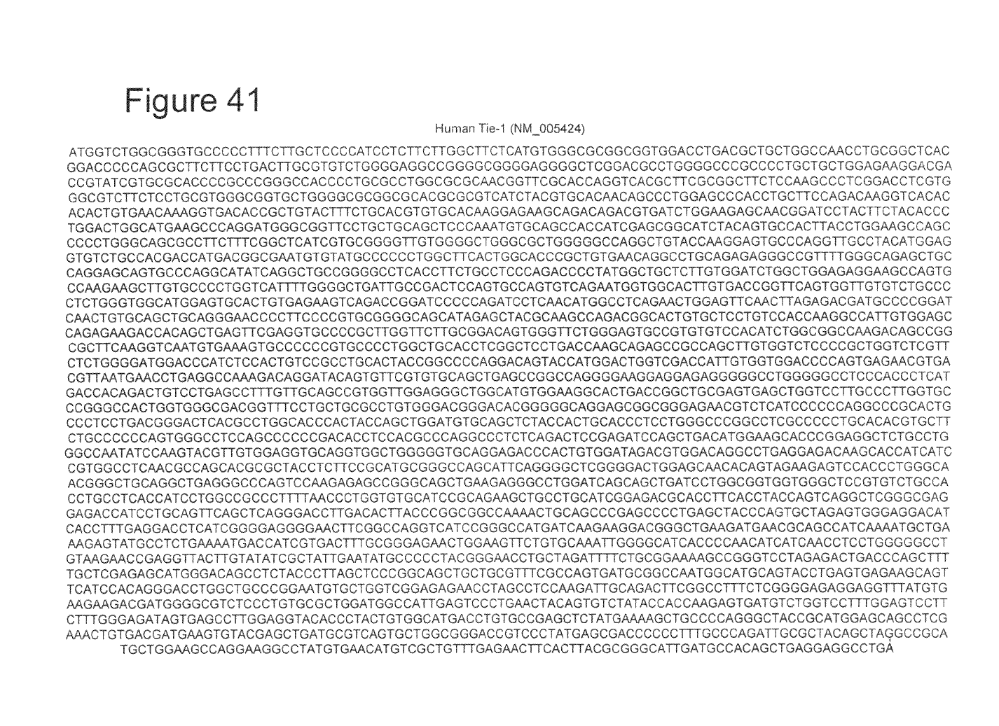

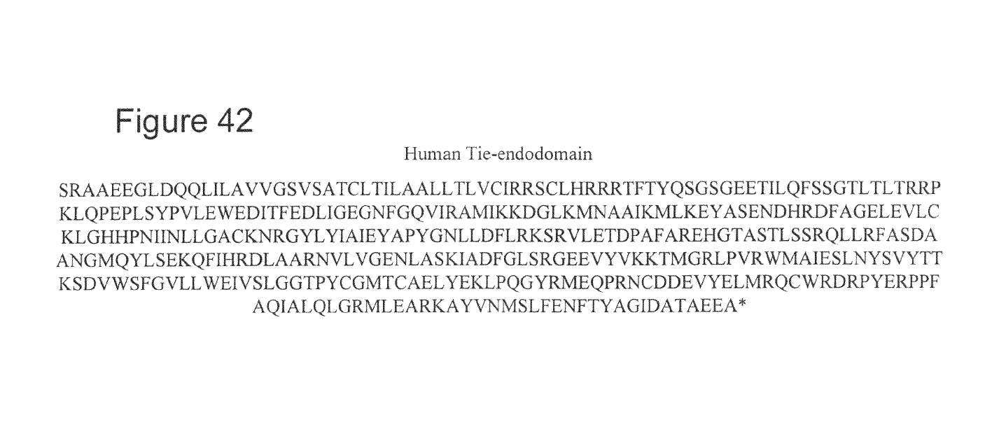

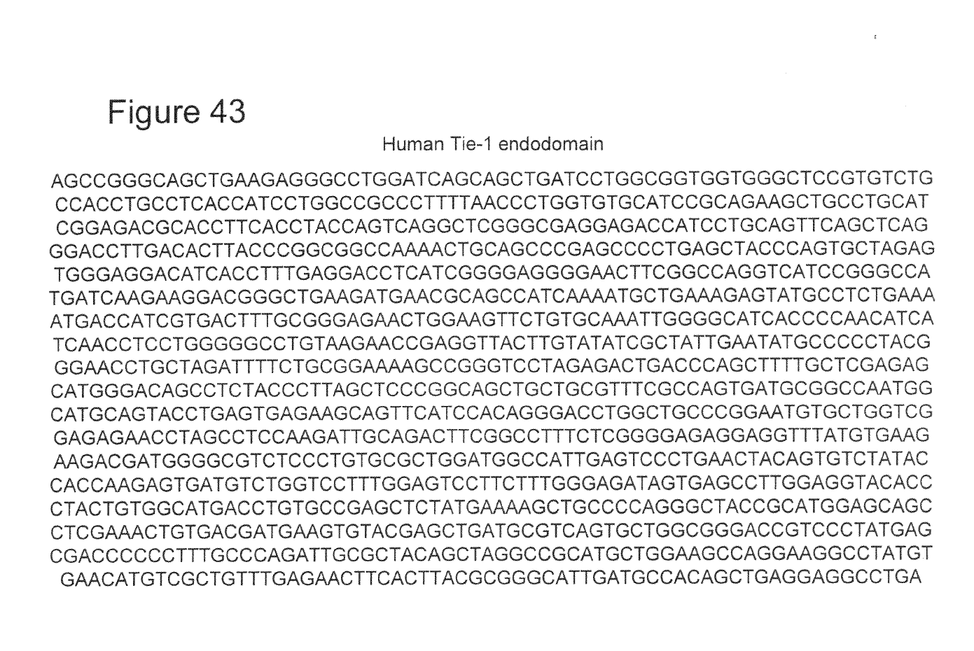

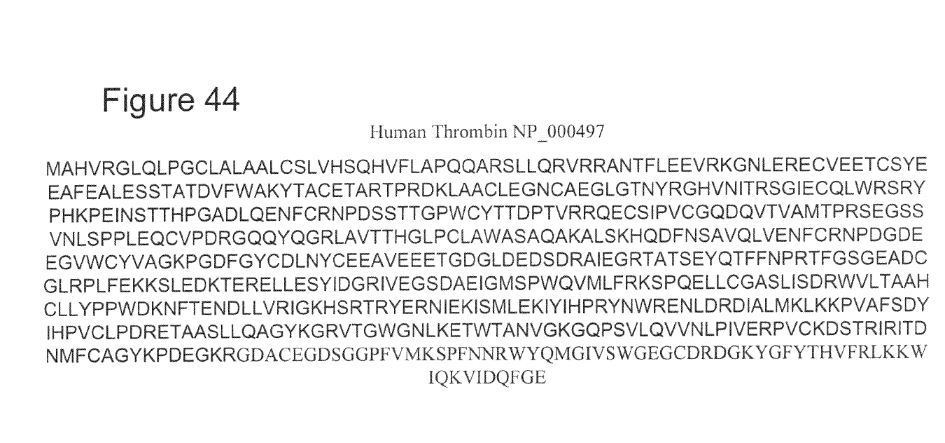

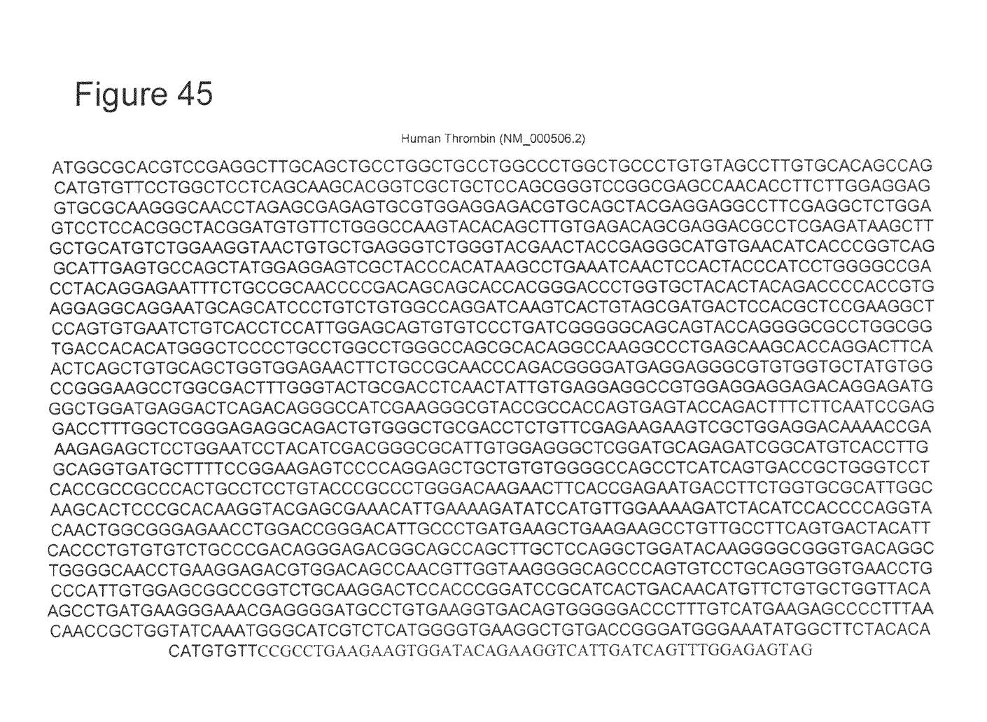

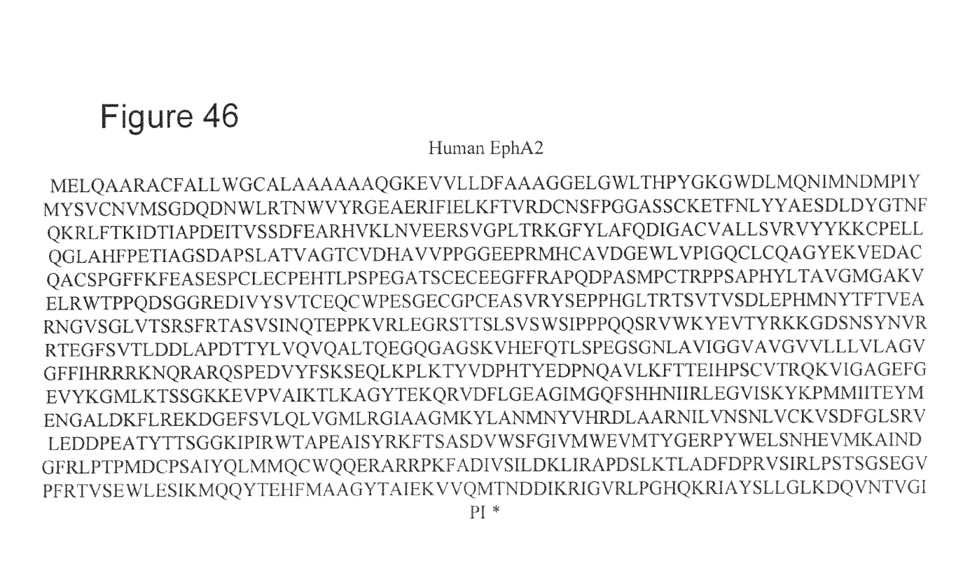

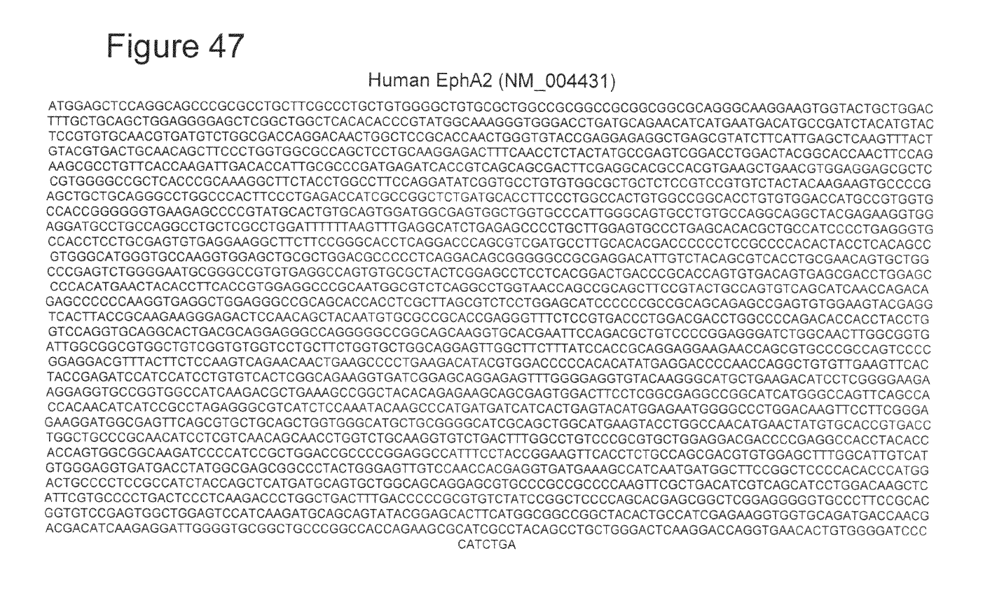

Taken together, the experiments described in the above examples show that thrombin transactivates 12 receptor tyrosine kinases in endothelial cells. We have focused our studies on EphA2 and one model for how trans activation of EphA2 mediates thrombin-induced ICAM-1 upregulation is shown in FIG. 39. Thrombin activates the PAR-1, which in turn activates a Src-family kinase to cause tyrosine phosphorylation of EphA2. Transactivation of EphA2 leads to PI3 kinase, Crk1, RASGAP-1, and SHP-2 association. Any of these pathways, singly or in combination, may activate NFκB, resulting in ICAM-1 expression. Increased expression of ICAM-1 promotes attachment of leukoctyes to the endothelium.

### Example 21

### Assay for Identification of Additional Substrates of EphA2

To assay for additional ligands or substrates of EphA2, physical interactions between a candidate protein (e.g., PI3 kinase or SHP-2) with activated EphA2 can be identified by co-immunoprecipitation and western blot analysis. Monolayer HUVECs are stimulated with thrombin (1 U/ml) for 2, 5, 15, 30, 60 mins. Cell lysates are prepared as described above in an NP-40 lysis buffer (25 mM TrisHCl, pH 7.5, 2.7 mM KCl, 137 mM NaCl, 10% glycerol, 1% NP40, 5 mM EDTA, 10 mM NaF, 1 mM sodium orthovanadate) supplemented with 1 tablet of protease inhibitor cocktail (Roche). NP-40 is chosen because it is a non-ionic detergent, which often yields better results in co-immunoprecipitation experiments. EphA2 is first pulled down by a goat polyclonal anti human EphA2 antibody from R&D and Protein A/G Plus (Santa Cruz). This antibody is chosen because it immunoprecipitates EphA2 from HUVECs in the NP-40 lysis buffer very efficiently. In addition, the antibody targets the extracellular domain of EphA2 and should minimally affect potential interactions between intracellular domain of EphA2 and cytosolic proteins. Normal goat IgG is used to control for non-specific interactions. Western blot is then performed. Examples of antibodies that can be used to test, for example, for PI3K and SHP-2 interaction include: p85a-specific (Santa Cruz, SC-71894), p85(3-specific (Santa Cruz, SC-56934), and SHP-2 (Cell Signal 3752). The interactions are predicted to cease at 60 mins post stimulation, since EphA2 tyrosine phosphorylation returns to basal level.

SiRNA methods can also be used to knockdown expression of the protein of interest to determine the funcutional consequence of the interaction. For simplicity, PI3 kinase p85a is used to illustrate our approach. Two validated Stealth siRNA against human PI3 kinase p85a are purchased from Invitrogen (#1293749). Stealth siRNA transfection, thrombin stimulation, and ICAM-1 detection are performed as described above.

If both PI3 kinase and SHP-2 are required for ICAM-1 expression induced by thrombin, SHP-2 can be knocked down by siRNA as well. Then the activity of PI3 kinase induced by thrombin can be determined using Akt phosphorylation as a surrogate marker.

### OTHER EMBODIMENTS

From the foregoing description, it will be apparent that variations and modifications may be made to the invention described herein to adopt it to various usages and conditions. Such embodiments are also within the scope of the following claims.

All publications, patent applications, and patents mentioned in this specification, including U.S. Provisional Application No. 60/879,908, filed Jan. 11, 2007, are herein incorporated by reference to the same extent as if each independent publication, patent application, or patent was specifically and individually indicated to be incorporated by reference.

From the foregoing description, one skilled in the art can easily ascertain the essential characteristics of this invention; can make various changes and modifications of the invention to adapt it to various usages and conditions. Thus, other embodiments are also within the claims.

What is claimed is:

1. A method of treating or preventing atherosclerosis in a subject, said method comprising administering to said subject a therapeutically effective amount of a Tie-1 inhibitor compound in an amount and for a time sufficient to treat or prevent said atherosclerosis in said subject.

2. The method of claim 1, wherein said Tie-1 inhibitor compound reduces or inhibits the biological activity or expression levels of a Tie-1 protein or nucleic acid molecule.

3. The method of claim 2, wherein said biological activity of a Tie-1 protein is selected from the group consisting of kinase activity; cleavage of Tie-1; shedding of the Tie-1 ectodomain; phosphorylation of the Tie-1 endodomain; increased endothelial cell adhesion; increased smooth muscle cell migration; inhibition of eNOS expression or biological activity; and activation of one or more cytokine or inflammatory markers.

4. The method of claim 1, further comprising administering to said subject a therapeutically effective amount of one or more compounds that inhibit the expression level or biological activity of one or more of the following compounds: tissue factor, thrombin, IP-10, G-CFS, IL-6, VCAM-1, ICAM-1, CCL20, CCL2, CXCL5, E-selectin, soluble CD44, p38 MAP kinase, EGFR, insulin receptor, IGF-IR, AXL, HGFR, Flt-1, KDR (VEGFR2), VEGFR2 endodomain, c-RET, MER, EphA2, and Tie-2; or a compound that increases the expression level or biological activity of nitric oxide synthase (eNOS).

5. The method of claim 1, wherein said Tie-1 inhibitor is an shRNA molecule.

6. The method of claim 5, wherein said shRNA comprises a nucleic acid sequence selected from the group consisting of SEQ ID NO:16, 17, and 18.

7. The method of claim 1, wherein said Tie-1 inhibitor is selected from the group consisting of a small molecule chemical compound, an antibody, and a polypeptide, or fragment thereof.

8. The method of claim 7, wherein said polypeptide is fragment of Tie-1.


SEQUENCE LISTING (44 sequences)

SEQ ID NO: 1 - protein, 1356 aa, Homo sapien [sequence omitted]

SEQ ID NO: 2 - DNA, 4071 bp, Homo sapien
atggagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaa

SEQ ID NO: 3 - protein, 1138 aa, Homo sapiens
MVWRVPPFLLPILFLASHVGAAVDLTLLANLRLTDPQRFFLTCVSGEAGAGRGSDAWGPPLLLEKDDRIVRTPPGPPLRLARNGSHQVTLRGFSKPSDLVGVFSCVGGAGARRTRVIYVHNSPGAHLLPDKVTHTVNKGDTAVLSARVHKEKQTDVIWKSNGSYFYTLDWHEAQDGRFLLQLPNVQPPSSGIYSATYLEASPLGSAFFRLIVRGCGAGRWGPGCTKECPGCLHGGVCHDHDGECVCPPGFTGTRCEQACREGRFGQSCQEQCPGISGCRGLTFCLPDPYGCSCGSGWRGSQCQEACAPGHFGADCRLQCQCQNGGTCDRFSGCVCPSGWHGVHCEKSDRIPQILNMASELEFNLETMPRINCAAAGNPFPVRGSIELRKPDGTVLLSTKAIVEPEKTTAEFEVPRLVLADSGFWECRVSTSGGQDSRRFKVNVKVPPVPLAAPRLLTKQSRQLVVSPLVSFSGDGPISTVRLHYRPQDSTMDWSTIVVDPSENVTLMNLRPKTGYSVRVQLSRPGEGGEGAWGPPTLMTTDCPEPLLQPWLEGWHVEGTDRLRVSWSLPLVPGPLVGDGFLLRLWDGTRGQERRENVSSPQARTALLTGLTPGTHYQLDVQLYHCTLLGPASPPAHVLLPPSGPPAPRHLHAQALSDSEIQLTWKHPEALPGPISKYVVEVQVAGGAGDPLWIDVDRPEETSTIIRGLNASTRYLFRMRASIQGLGDWSNTVEESTLGNGLQAEGPVQESRAAEEGLDQQLILAVVGSVSATCLTILAALLTLVCIRRSCLHRRRTFTYQSGSGEETILQFSSGTLTLTRRPKLQPEPLSYPVLEWEDITFEDLIGEGNFGQVIRAMIKKDGLKMNAAIKMLKEYASENDHRDFAGELEVLCKLGHHPNIINLLGACKNRGYLYIAIEYAPYGNLLDFLRKSRVLETDPAFAREHGTASTLSSRQLLRFASDAANGMQYLSEKQFIHRDLAARNVLVGENLASKIADFGLSRGEEVYVKKTMGRLPVRWMAIESLNYSVYTTKSDVWSFGVLLWEIVSLGGTPYCGMTCAELYEKLPQGYRMEQPRNCDDEVYELMRQCWRDRPYERPPFAQIALQLGRMLEARKAYVNMSLFENFTYAGIDATAEEA

SEQ ID NO: 4 - DNA, 3417 bp, Homo sapiens
atggtctggcgggtgccccctttcttgctccccatcctcttcttggcttctcatgtgggcgcggcggtggacctgacgctgctggccaacctgcggctcacggacccccagcgcttcttcctgacttgcgtgtctggggaggccggggcggggaggggctcggacgcctggggcccgcccctgctgctggagaaggacgaccgtatcgtgcgcaccccgcccgggccacccctgcgcctggcgcgcaacggttcgcaccaggtcacgcttcgcggcttctccaagccctcggacctcgtgggcgtcttctcctgcgtgggcggtgctggggcgcggcgcacgcgcgtcatctacgtgcacaacagccctggagcccacctgcttccagacaaggtcacacacactgtgaacaaaggtgacaccgctgtactttctgcacgtgtgcacaaggagaagcagacagacgtgatctggaagagcaacggatcctacttctacaccctggactggcatgaagcccaggatgggcggttcctgctgcagctcccaaatgtgcagccaccatcgagcggcatctacagtgccacttacctggaagccagccccctgggcagcgccttctttcggctcatcgtgcggggttgtggggctgggcgctgggggccaggctgtaccaaggagtgcccaggttgcctacatggaggtgtctgccacgaccatgacggcgaatgtgtatgcccccctggcttcactggcacccgctgtgaacaggcctgcagagagggccgttttgggcagagctgccaggagcagtgcccaggcatatcaggctgccggggcctcaccttctgcctcccagacccctatggctgctcttgtggatctggctggagaggaagccagtgccaagaagcttgtgcccctggtcattttggggctgattgccgactccagtgccagtgtcagaatggtggcacttgtgaccggttcagtggttgtgtctgcccctctgggtggcatggagtgcactgtgagaagtcagaccggatcccccagatcctcaacatggcctcagaactggagttcaacttagagacgatgccccggatcaactgtgcagctgcagggaaccccttccccgtgcggggcagcatagagctacgcaagccagacggcactgtgctcctgtccaccaaggccattgtggagccagagaagaccacagctgagttcgaggtgccccgcttggttcttgcggacagtgggttctgggagtgccgtgtgtccacatctggcggccaagacagccggcgcttcaaggtcaatgtgaaagtgccccccgtgcccctggctgcacctcggctcctgaccaagcagagccgccagcttgtggtctccccgctggtctcgttctctggggatggacccatctccactgtccgcctgcactaccggccccaggacagtaccatggactggtcgaccattgtggtggaccccagtgagaacgtgacgttaatgaacctgaggccaaagacaggatacagtgttcgtgtgcagctgagccggccaggggaaggaggagagggggcctgggggcctcccaccctcatgaccacagactgtcctgagcctttgttgcagccgtggttggagggctggcatgtggaaggcactgaccggctgcgagtgagctggtccttgcccttggtgcccgggccactggtgggcgacggtttcctgctgcgcctgtgggacgggacacgggggcaggagcggcgggagaacgtctcatccccccaggcccgcactgccctcctgacgggactcacgcctggcacccactaccagctggatgtgcagctctaccactgcaccctcctgggcccggcctcgccccctgcacacgtgcttctgccccccagtgggcctccagccccccgacacctccacgcccaggccctctcagactccgagatccagctgacatggaagcacccggaggctctgcctgggccaatatccaagtacgttgtggaggtgcaggtggctgggggtgcaggagacccactgtggatagacgtggacaggcctgaggagacaagcaccatcatccgtggcctcaacgccagcacgcgctacctcttccgcatgcgggccagcattcaggggctcggggactggagcaacacagtagaagagtccaccctgggcaacgggctgcaggctgagggcccagtccaagagagccgggcagctgaagagggcctggatcagcagctgatcctggcggtggtgggctccgtgtctgccacctgcctcaccatcctggccgcccttttaaccctggtgtgcatccgcagaagctgcctgcatcggagacgcaccttcacctaccagtcaggctcgggcgaggagaccatcctgcagttcagctcagggaccttgacacttacccggcggccaaaactgcagcccgagcccctgagctacccagtgctagagtgggaggacatcacctttgaggacctcatcggggaggggaacttcggccaggtcatccgggccatgatcaagaaggacgggctgaagatgaacgcagccatcaaaatgctgaaagagtatgcctctgaaaatgaccatcgtgactttgcgggagaactggaagttctgtgcaaattggggcatcaccccaacatcatcaacctcctgggggcctgtaagaaccgaggttacttgtatatcgctattgaatatgccccctacgggaacctgctagattttctgcggaaaagccgggtcctagagactgacccagcttttgctcgagagcatgggacagcctctacccttagctcccggcagctgctgcgtttcgccagtgatgcggccaatggcatgcagtacctgagtgagaagcagttcatccacagggacctggctgcccggaatgtgctggtcggagagaacctagcctccaagattgcagacttcggcctttctcggggagaggaggtttatgtgaagaagacgatggggcgtctccctgtgcgctggatggccattgagtccctgaactacagtgtctataccaccaagagtgatgtctggtcctttggagtccttctttgggagatagtgagccttggaggtacaccctactgtggcatgacctgtgccgagctctatgaaaagctgccccagggctaccgcatggagcagcctcgaaactgtgacgatgaagtgtacgagctgatgcgtcagtgctggcgggaccgtccctatgagcgacccccctttgcccagattgcgctacagctaggccgcatgctggaagccaggaaggcctatgtgaacatgtcgctgtttgagaacttcacttacgcgggcattgatgccacagctgaggaggcctga

SEQ ID NO: 5 - protein, 389 aa, Homo sapiens
SRAAEEGLDQQLILAVVGSVSATCLTILAALLTLVCIRRSCLHRRRTFTYQSGSGEETILQFSSGTLTLTRRPKLQPEPLSYPVLEWEDITFEDLIGEGNFGQVIRAMIKKDGLKMNAAIKMLKEYASENDHRDFAGELEVLCKLGHHPNIINLLGACKNRGYLYIAIEYAPYGNLLDFLRKSRVLETDPAFAREHGTASTLSSRQLLRFASDAANGMQYLSEKQFIHRDLAARNVLVGENLASKIADFGLSRGEEVYVKKTMGRLPVRWMAIESLNYSVYTTKSDVWSFGVLLWEIVSLGGTPYCGMTCAELYEKLPQGYRMEQPRNCDDEVYELMRQCWRDRPYERPPFAQIALQLGRMLEARKAYVNMSLFENFTYAGIDATAEEA

SEQ ID NO: 6 - DNA, 1170 bp, Homo sapiens
agccgggcagctgaagagggcctggatcagcagctgatcctggcggtggtgggctccgtgtctgccacctgcctcaccatcctggccgcccttttaaccctggtgtgcatccgcagaagctgcctgcatcggagacgcaccttcacctaccagtcaggctcgggcgaggagaccatcctgcagttcagctcagggaccttgacacttacccggcggccaaaactgcagcccgagcccctgagctacccagtgctagagtgggaggacatcacctttgaggacctcatcggggaggggaacttcggccaggtcatccgggccatgatcaagaaggacgggctgaagatgaacgcagccatcaaaatgctgaaagagtatgcctctgaaaatgaccatcgtgactttgcgggagaactggaagttctgtgcaaattggggcatcaccccaacatcatcaacctcctgggggcctgtaagaaccgaggttacttgtatatcgctattgaatatgccccctacgggaacctgctagattttctgcggaaaagccgggtcctagagactgacccagcttttgctcgagagcatgggacagcctctacccttagctcccggcagctgctgcgtttcgccagtgatgcggccaatggcatgcagtacctgagtgagaagcagttcatccacagggacctggctgcccggaatgtgctggtcggagagaacctagcctccaagattgcagacttcggcctttctcggggagaggaggtttatgtgaagaagacgatggggcgtctccctgtgcgctggatggccattgagtccctgaactacagtgtctataccaccaagagtgatgtctggtcctttggagtccttctttgggagatagtgagccttggaggtacaccctactgtggcatgacctgtgccgagctctatgaaaagctgccccagggctaccgcatggagcagcctcgaaactgtgacgatgaagtgtacgagctgatgcgtcagtgctggcgggaccgtccctatgagcgacccccctttgcccagattgcgctacagctaggccgcatgctggaagccaggaaggcctatgtgaacatgtcgctgtttgagaacttcacttacgcgggcattgatgccacagctgaggaggcctga

SEQ ID NO: 7 - protein, 622 aa, Homo sapiens
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 8 - DNA, 1869 bp, Homo sapiens
atggcgcacgtccgaggcttgcagctgcctggctgcctggccctggctgccctgtgtagccttgtgcacagccagcatgtgttcctggctcctcagcaagcacggtcgctgctccagcgggtccggcgagccaacaccttcttggaggaggtgcgcaagggcaacctagagcgagagtgcgtggaggagacgtgcagctacgaggaggccttcgaggctctggagtcctccacggctacggatgtgttctgggccaagtacacagcttgtgagacagcgaggacgcctcgagataagcttgctgcatgtctggaaggtaactgtgctgagggtctgggtacgaactaccgagggcatgtgaacatcacccggtcaggcattgagtgccagctatggaggagtcgctacccacataagcctgaaatcaactccactacccatcctggggccgacctacaggagaatttctgccgcaaccccgacagcagcaccacgggaccctggtgctacactacagaccccaccgtgaggaggcaggaatgcagcatccctgtctgtggccaggatcaagtcactgtagcgatgactccacgctccgaaggctccagtgtgaatctgtcacctccattggagcagtgtgtccctgatcgggggcagcagtaccaggggcgcctggcggtgaccacacatgggctcccctgcctggcctgggccagcgcacaggccaaggccctgagcaagcaccaggacttcaactcagctgtgcagctggtggagaacttctgccgcaacccagacggggatgaggagggcgtgtggtgctatgtggccgggaagcctggcgactttgggtactgcgacctcaactattgtgaggaggccgtggaggaggagacaggagatgggctggatgaggactcagacagggccatcgaagggcgtaccgccaccagtgagtaccagactttcttcaatccgaggacctttggctcgggagaggcagactgtgggctgcgacctctgttcgagaagaagtcgctggaggacaaaaccgaaagagagctcctggaatcctacatcgacgggcgcattgtggagggctcggatgcagagatcggcatgtcaccttggcaggtgatgcttttccggaagagtccccaggagctgctgtgtggggccagcctcatcagtgaccgctgggtcctcaccgccgcccactgcctcctgtacccgccctgggacaagaacttcaccgagaatgaccttctggtgcgcattggcaagcactcccgcacaaggtacgagcgaaacattgaaaagatatccatgttggaaaagatctacatccaccccaggtacaactggcgggagaacctggaccgggacattgccctgatgaagctgaagaagcctgttgccttcagtgactacattcaccctgtgtgtctgcccgacagggagacggcagccagcttgctccaggctggatacaaggggcgggtgacaggctggggcaacctgaaggagacgtggacagccaacgttggtaaggggcagcccagtgtcctgcaggtggtgaacctgcccattgtggagcggccggtctgcaaggactccacccggatccgcatcactgacaacatgttctgtgctggttacaagcctgatgaagggaaacgaggggatgcctgtgaaggtgacagtgggggaccctttgtcatgaagagcccctttaacaaccgctggtatcaaatgggcatcgtctcatggggtgaaggctgtgaccgggatgggaaatatggcttctacacacatgtgttccgcctgaagaagtggatacagaaggtcattgatcagtttggagagtag

SEQ ID NO: 9 - protein, 976 aa, Homo sapiens
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 10 - DNA, 2931 bp, Homo sapiens
atggagctccaggcagcccgcgcctgcttcgccctgctgtggggctgtgcgctggccgcggccgcggcggcgcagggcaaggaagtggtactgctggactttgctgcagctggaggggagctcggctggctcacacacccgtatggcaaagggtgggacctgatgcagaacatcatgaatgacatgccgatctacatgtactccgtgtgcaacgtgatgtctggcgaccaggacaactggctccgcaccaactgggtgtaccgaggagaggctgagcgtatcttcattgagctcaagtttactgtacgtgactgcaacagcttccctggtggcgccagctcctgcaaggagactttcaacctctactatgccgagtcggacctggactacggcaccaacttccagaagcgcctgttcaccaagattgacaccattgcgcccgatgagatcaccgtcagcagcgacttcgaggcacgccacgtgaagctgaacgtggaggagcgctccgtggggccgctcacccgcaaaggcttctacctggccttccaggatatcggtgcctgtgtggcgctgctctccgtccgtgtctactacaagaagtgccccgagctgctgcagggcctggcccacttccctgagaccatcgccggctctgatgcaccttccctggccactgtggccggcacctgtgtggaccatgccgtggtgccaccggggggtgaagagccccgtatgcactgtgcagtggatggcgagtggctggtgcccattgggcagtgcctgtgccaggcaggctacgagaaggtggaggatgcctgccaggcctgctcgcctggattttttaagtttgaggcatctgagagcccctgcttggagtgccctgagcacacgctgccatcccctgagggtgccacctcctgcgagtgtgaggaaggcttcttccgggcacctcaggacccagcgtcgatgccttgcacacgacccccctccgccccacactacctcacagccgtgggcatgggtgccaaggtggagctgcgctggacgccccctcaggacagcgggggccgcgaggacattgtctacagcgtcacctgcgaacagtgctggcccgagtctggggaatgcgggccgtgtgaggccagtgtgcgctactcggagcctcctcacggactgacccgcaccagtgtgacagtgagcgacctggagccccacatgaactacaccttcaccgtggaggcccgcaatggcgtctcaggcctggtaaccagccgcagcttccgtactgccagtgtcagcatcaaccagacagagccccccaaggtgaggctggagggccgcagcaccacctcgcttagcgtctcctggagcatccccccgccgcagcagagccgagtgtggaagtacgaggtcacttaccgcaagaagggagactccaacagctacaatgtgcgccgcaccgagggtttctccgtgaccctggacgacctggccccagacaccacctacctggtccaggtgcaggcactgacgcaggagggccagggggccggcagcaaggtgcacgaattccagacgctgtccccggagggatctggcaacttggcggtgattggcggcgtggctgtcggtgtggtcctgcttctggtgctggcaggagttggcttctttatccaccgcaggaggaagaaccagcgtgcccgccagtccccggaggacgtttacttctccaagtcagaacaactgaagcccctgaagacatacgtggacccccacacatatgaggaccccaaccaggctgtgttgaagttcactaccgagatccatccatcctgtgtcactcggcagaaggtgatcggagcaggagagtttggggaggtgtacaagggcatgctgaagacatcctcggggaagaaggaggtgccggtggccatcaagacgctgaaagccggctacacagagaagcagcgagtggacttcctcggcgaggccggcatcatgggccagttcagccaccacaacatcatccgcctagagggcgtcatctccaaatacaagcccatgatgatcatcactgagtacatggagaatggggccctggacaagttccttcgggagaaggatggcgagttcagcgtgctgcagctggtgggcatgctgcggggcatcgcagctggcatgaagtacctggccaacatgaactatgtgcaccgtgacctggctgcccgcaacatcctcgtcaacagcaacctggtctgcaaggtgtctgactttggcctgtcccgcgtgctggaggacgaccccgaggccacctacaccaccagtggcggcaagatccccatccgctggaccgccccggaggccatttcctaccggaagttcacctctgccagcgacgtgtggagctttggcattgtcatgtgggaggtgatgacctatggcgagcggccctactgggagttgtccaaccacgaggtgatgaaagccatcaatgatggcttccggctccccacacccatggactgcccctccgccatctaccagctcatgatgcagtgctggcagcaggagcgtgcccgccgccccaagttcgctgacatcgtcagcatcctggacaagctcattcgtgcccctgactccctcaagaccctggctgactttgacccccgcgtgtctatccggctccccagcacgagcggctcggagggggtgcccttccgcacggtgtccgagtggctggagtccatcaagatgcagcagtatacggagcacttcatggcggccggctacactgccatcgagaaggtggtgcagatgaccaacgacgacatcaagaggattggggtgcggctgcccggccaccagaagcgcatcgcctacagcctgctgggactcaaggaccaggtgaacactgtggggatccccatctga

SEQ ID NO: 11 - protein, 5 aa, Artificial Sequence
  Synthetic
TFLLR

SEQ ID NO: 12 - protein, 5 aa, Artificial Sequence
  Synthetic
RLLFT

SEQ ID NO: 13 - protein, 6 aa, Artificial Sequence
  Synthetic
SLIGKV

SEQ ID NO: 14 - protein, 6 aa, Artificial Sequence
  Synthetic
GYPGKF

SEQ ID NO: 15 - DNA, 22 bp, Artificial Sequence
  synthetic
aggccaggatgtgtcaaggatt

SEQ ID NO: 16 - DNA, 22 bp, Artificial Sequence
  synthetic
aatccttgacacatcctggccc

SEQ ID NO: 17 - DNA, 22 bp, Artificial Sequence
  synthetic
ccgcagccatcaagatgctaaa

SEQ ID NO: 18 - DNA, 22 bp, Artificial Sequence
  synthetic
tttagcatcttgatggctgcgt

SEQ ID NO: 19 - DNA, 22 bp, Artificial Sequence
  synthetic
accagtgagaatgtgacattaa

SEQ ID NO: 20 - DNA, 22 bp, Artificial Sequence
  synthetic
ttaatgtcgcattctcactggg

SEQ ID NO: 21 - DNA, 22 bp, Artificial Sequence
  synthetic
aggccagctgatgccgagtaaa

SEQ ID NO: 22 - DNA, 22 bp, Artificial Sequence
  synthetic
tttactcggcatcagctggccg

SEQ ID NO: 23 - DNA, 22 bp, Artificial Sequence
  synthetic
aggccttctccgccatcttctt

SEQ ID NO: 24 - DNA, 22 bp, Artificial Sequence
  synthetic
aagaagatggcggagaaggccg

SEQ ID NO: 25 - DNA, 22 bp, Artificial Sequence
  synthetic
ccctgaataacagcatatacaa

SEQ ID NO: 26 - DNA, 22 bp, Artificial Sequence
  synthetic
ttgtatatgctgttattcaggt

SEQ ID NO: 27 - DNA, 25 bp, Artificial Sequence
  synthetic
gatgctgaaaaatggcaaatccaac

SEQ ID NO: 28 - DNA, 25 bp, Artificial Sequence
  synthetic
tgatgttcaaggaagagaaaacaac

SEQ ID NO: 29 - DNA, 25 bp, Artificial Sequence
  synthetic
ggggctctgttcccaggacctggca

SEQ ID NO: 30 - DNA, 25 bp, Artificial Sequence
  synthetic
gtggcattcaaggagtacctctctc

SEQ ID NO: 31 - DNA, 25 bp, Artificial Sequence
  synthetic
gtgtgagcaaattgtgaactgtaca

SEQ ID NO: 32 - DNA, 25 bp, Artificial Sequence
  synthetic
ggattcaatgaggagacttgcctgg

SEQ ID NO: 33 - RNA, 25 nt, Artificial Sequence
  Synthetic
gcaaggaagugguacugcuggacuu

SEQ ID NO: 34 - RNA, 25 nt, Artificial Sequence
  Synthetic
gggaccugaugcagaacaucaugaa

SEQ ID NO: 35 - DNA, 1443 bp, Artificial Sequence
  synthetic
ctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccatccaggcaactcctagtagtacaacggaagaaacagctacccagaaggaacagtggtttggcaacagatggcatgagggatatcgccaaacacccaaagaagactcccattcgacaacagggacagctgcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcagtaaggattataaaacctagttggcttcagctattgataagaatcaatcaattatgggtacttttgcagtgtctttggtggaggtgcatccattagctgccgttacactgttacttttaatcaaaaggtgcg

SEQ ID NO: 36 - DNA, 1476 bp, Artificial Sequence
  synthetic
tcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagtacgtcttcaaataccatctcagcaggctgggagccaaatgaagaaaatgaagatgaaagagacagacacctcagtttttctggatcaggcattgatgatgatgaagattttatctccagcaccattatctgcctcttcacccggaggatctacaaacagcacacagtgactaagtccctgggctttcaagtgcagagggatactacagactgcatggatgggcaaaatggcgcctttggttatcctcggtggagggctggtgttttcaaagctgtccttcctactgctgcagcttctttgactgttttatctggaagatctcatgttctgaaccccaaggtattctatgacagaatgcaaaggacactgagatgcttaccaatatggctgaattaagctgaagtcaccctccgctttccctccatttccatgcagccatctatacaacctggtatagatgattcattgcatagcctacagaagcaagactatgtgcgggacaagtggcgaaaggctcaagaaataacatttttctaattgcttcaatcatcgttatcacagtttcaaccacaccacgggcttttgaccacacaaaacagaacaggactggacccagtggaacccaagccattcaatcgagtgctacttcagacacacaagatgactggtatgggtctgcatatttatgaagatttttcccctcaagccatgatgctgcaaggtcaatcatcgagccaatggaaaattcaggattagatc

SEQ ID NO: 37 - protein, 379 aa, Homo sapien
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSSKDYKT

SEQ ID NO: 38 - protein, 742 aa, Homo sapien
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 39 - protein, 699 aa, Homo sapien
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATSTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 40 - protein, 493 aa, Homo sapien
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATNMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 41 - protein, 361 aa, Homo sapien
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATRDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 42 - protein, 139 aa, Homo sapien
MDKFWWHAAWGLCLVPLSLAQIDLNITCRFAGVFHVEKNGRYSISRTEAADLCKAFNSTLPTMAQMEKALSIGFETCSLHCSQQSKKVWAEEKASDQQWQWSCGGQKAKWTQRRGQQVSGNGAFGEQGVVRNSRPVYDS

SEQ ID NO: 43 - protein, 80 aa, Homo sapien
MDKFWWHAAWGLCLVPLSLAQIDLNITCRFAGVFHVEKNGRYSISRTEAADLCKAFNSTLPTMAQMEKALSIGFETCSST

SEQ ID NO: 44 - RNA, 63 nt, Mus musculus
aggccaggaugugucaaggauuuagugaagccacagauguaaauccuugacacauccuggccg